(19) United States
(12) Patent Application Publication
(10) Pub. No.: US 2025/0188163 A1
(43) Pub. Date: Jun. 12, 2025

(54) GPRC5D ANTIBODIES WITH ENHANCED EFFECTOR FUNCTION AND USES THEREOF

(71) Applicant: Janssen Biotech, Inc., Horsham, PA (US)

(72) Inventors: Bradley Heidrich, Horsam, PA (US); Xiefan LIN-SCHMIDT, Raritan, NJ (US); James Salvatore TESTA, JR., Raritan, NJ (US); John PATRICK, Raritan, NJ (US); Brian J. GEIST, Raritan, NJ (US); Sheng-Jiun WU, Raritan, NJ (US); Adam ZWOLAK, Raritan, NJ (US); Ulrike PHILIPPAR, Beerse (BE)

(21) Appl. No.: 18/972,307

(22) Filed: Dec. 6, 2024

Related U.S. Application Data
(60) Provisional application No. 63/607,729, filed on Dec. 8, 2023.

(51) Int. Cl. C07K 16/28; A61K 39/00; A61P 35/00

(52) U.S. Cl. CPC C07K 16/28; A61P 35/00; A61K 2039/505; C07K 2317/35; C07K 2317/73; C07K 2317/732; C07K 2317/734; C07K 2317/92; C07K 2317/94

(57) ABSTRACT

Herein are antibodies or antigen-binding fragments specifically binding to GPRC5D. Additionally, monovalent antibodies or antigen-binding fragments specifically binding to GPRC5D are also included. The Fc region of these antibodies contains K248E and T437R mutations (designated as “RE mutations”) per the EU numbering system. The described antibodies, expressed in host cells that lack fucosylation capabilities, exhibit enhanced antibody-dependent cellular cytotoxicity (ADCC) and enhanced complement-dependent cytotoxicity (CDC).

### SEQUENCE LISTING

The instant application contains a Sequence Listing which has been submitted electronically in XML file format and is hereby incorporated by reference in its entirety. Said XML copy, created on Nov. 27, 2024, is named JBI6863WOPCT1_SL.xml and is 29,195 bytes in size.

### TECHNICAL FIELD

Provided herein are GPRC5D antibodies with enhanced antibody-dependent cellular cytotoxicity (ADCC) and enhanced complement-dependent cytotoxicity (CDC).

### BACKGROUND

Over the past decade, the treatment landscape for multiple myeloma (MM) has undergone significant changes, particularly with the development of effective therapeutic combinations. Combinations involving proteasome inhibitors (PIs), immunomodulatory drugs (IMiDs), alkylators, biologics like monoclonal antibodies (mAbs), bispecific T cell redirection antibodies, and chimeric antigen receptor T cells (CARTs) have vastly expanded treatment options for MM patients (Kumar S, Baizer L, Callander NS, et al. Gaps and opportunities in the treatment of relapsed-refractory multiple myeloma: Consensus recommendations of the NCI Multiple Myeloma Steering Committee. Blood Cancer J. 2022; 12 (6): 98. Published 2022 Jun. 29. doi: 10.1038/s41408-022-00695-5). Despite the introduction of new therapies for MM, patients still experience relapses, emphasizing the necessity for the development of forward-thinking novel therapeutics.

### SUMMARY

In one aspect, provided herein is an antibody or antigen-binding fragment specifically binding to GPRC5D comprising a heavy chain CDR1, a heavy chain CDR2, and a heavy chain CDR3 comprising the amino acid sequences of SEQ ID NO: 6, 7, and 8, respectively, and a light CDR1, a light chain CDR2, and a light chain CDR3 comprising the amino acid sequences of SEQ ID NO: 9, 10, and 11, respectively.

In some embodiments, the antibody or antigen-binding fragment comprises a heavy chain CDR1, a heavy chain CDR2, and a heavy chain CDR3 comprising the amino acid sequences of SEQ ID NO: 12, 13, and 8, respectively, and a light chain CDR1, a light chain CDR2, and a light chain CDR3 comprising the amino acid sequences of SEQ ID NO: 9, 10, and 11, respectively.

In some embodiments, the antibody or antigen-binding fragment comprises a heavy chain CDR1, a heavy chain CDR2, and a heavy chain CDR3 comprising the amino acid sequences of SEQ ID NO: 14, 15, and 8, respectively, and a light chain CDR1, a light chain CDR2, and a light chain CDR3 comprising the amino acid sequences of SEQ ID NO: 9, 10, and 11, respectively.

In some embodiments, the antibody or antigen-binding fragment comprises a heavy chain CDR1, a heavy chain CDR2, and a heavy chain CDR3 comprising the amino acid sequences of SEQ ID NO: 16, 17, and 18, respectively, and a light chain CDR1, a light chain CDR2, and a light chain CDR3 comprising the amino acid sequences of SEQ ID NO: 19, 20, and 11, respectively.

In some embodiments, the antibody or antigen-binding fragments comprises a heavy chain CDR1, a heavy chain CDR2, and a heavy chain CDR3 comprising the amino acid sequences of SEQ ID NO: 21, 22, and 23, respectively, and a light chain CDR1, a light chain CDR2, and a light chain CDR3 comprising the amino acid sequences of SEQ ID NO: 24, 25, and 26, respectively.

In some embodiments, the antibody or antigen-binding fragment comprises a heavy chain variable region (VH) comprising an amino acid sequence substantially the same or identical to SEQ ID NO: 1. In some embodiments, the antibody or antigen-binding fragment comprises a light chain variable region (VL) comprising an amino acid sequence substantially the same or identical to SEQ ID NO: 2. In some embodiments, the antibody or antigen-binding fragment comprises a VH comprising an amino acid sequence of SEQ ID NO: 1 and a VL comprising an amino acid sequence of SEQ ID NO: 2

In some embodiments, the antibody or antigen-binding fragment comprises an IgG1 isotype Fc region. In some embodiments, the IgG1 isotype Fc region comprises K248E and T437R (RE) mutations as per the EU numbering system.

In some embodiments, the antibody or the antigen-binding fragment is afucosylated.

In some embodiments, the antibody or the antigen-binding fragment has enhanced antibody-dependent cellular cytotoxicity (ADCC) activity and enhanced complement-dependent cytotoxicity (CDC) activity.

In some embodiments, the antibody or the antigen-binding fragment has antibody-dependent cellular phagocytosis (ADCP) activity.

In some embodiments, the antibody or the antigen-binding fragment further comprises knob-into-hole (KiH) mutations.

In some embodiments, the IgG1 isotype Fc region further comprises H435R and Y436F mutations per the EU numbering system.

Also provided is a monovalent antibody.

In some embodiments the monovalent antibody or antigen-binding fragment comprises an Fc domain and a Fab.

In some embodiments the monovalent antibody or antigen-binding fragment comprises a first heavy chain (HCl), a second heavy chain (HC2), and a second light chain (LC2).

In some embodiments the monovalent antibody or antigen-binding fragment comprises an HCl comprising SEQ ID NO: 3.

In some embodiments the monovalent antibody or antigen-binding fragment comprises an HC2 comprising SEQ ID NO: 4.

In some embodiments the monovalent antibody or antigen-binding fragment comprises an LC2 comprising SEQ ID NO: 5.

In some embodiments the monovalent antibody or antigen-binding fragment specifically binding to GPRC5D comprises an HCl comprising SEQ ID NO: 3, an HC2 comprising SEQ ID NO: 4 and an LC2 comprising SEQ ID NO: 5.

Also provided are synthetic polynucleotides encoding the antibodies, the monovalent antibodies or the antigen-binding fragments described herein.

Also provided are vectors comprising the synthetic polynucleotides encoding the antibodies, the monovalent antibodies or the antigen-binding fragments described herein.

Also provided are host cells comprising the vectors comprising the synthetic polynucleotide encoding the antibodies, the monovalent antibodies or the antigen-binding fragments described. In some embodiments, the host cells lack fucosylation capability.

In certain embodiments, provided is a pharmaceutical composition comprising the antibody, the monovalent antibody, or the antigen-binding fragment described herein and a pharmaceutically acceptable carrier.

Also provided are methods for generating the antibody, the monovalent antibody, or the antigen-binding fragment described herein. In some embodiments, the antibody, the monovalent antibody, or the antigen-binding fragment described herein is produced by the methods provided herein.

In some embodiments, the antibody, the monovalent antibody, or the antigen-binding fragment is produced by expressing the synthetic polynucleotide encoding the antibody, the monovalent antibody, or the antigen-binding fragment in a host cell that lacks afucosylation capability.

Also provided are methods of inhibiting the growth or proliferation of a multiple myeloma, comprising administering to a subject the antibody, the monovalent antibody, or the antigen-binding fragment described herein to inhibit the growth or proliferation of the multiple myeloma.

Also provided are methods of treating a multiple myeloma, comprising administering to a subject in need thereof the antibody, the monovalent antibody or the antigen-binding fragment described herein to the subject for a time sufficient to treat the multiple myeloma.

In some embodiments, the multiple myeloma is characterized by the presence of GPRC5D.

Also provided are kits comprising the antibody, the monovalent antibody, or the antigen-binding fragment described herein and packaging for the same.

In certain embodiments, provided are kits comprising the synthetic polynucleotide encoding the antibody, the monovalent antibody, or the antigen-binding fragment described herein and packaging for the same.

### BRIEF DESCRIPTION OF THE FIGURES

FIG. 1 illustrates a monovalent antibody targeting GPRC5D with an enhanced effector function. Fab, fragment antigen-binding; Fc, fragment crystallizable; GPRC5D, G-protein coupled receptor Family C Group 5 Member D; JAWA, Janssen Agonism Works Awesome, K248E and T437R; RF, H435R and Y436F.

FIG. 2 shows the impact of valency, JAWA mutations, and Afucoslyation on CDC.

FIG. 3 shows the CDC screening results of human anti-GPRC5D antibodies. MM.1R cells were incubated with human anti-GPRCSD antibodies in the presence of human serum for 24 hours. Cell viability was determined using CellTiter-Glo®. Data represent mean±SEM, 3 technical replicates.

FIG. 4 depicts the CDC screening results of rat anti-GPRC5D antibodies. MM.1R cells were incubated with rat anti-GPRC5D antibodies in the presence of human serum for 24 hours and cell viability was determined using CellTiter-Glo®. Data represent mean±SEM, 3 technical replicates.

FIG. 5 shows the CDC screening results of humanized anti-GPRC5D antibodies. MM.1R cells were incubated with humanized anti-GPRCSD antibodies in the presence of human serum for 24 hours and cell viability was determined using CellTiter-Glo®. Data represent mean±SEM, 3 technical replicates. Data represent mean±SEM, 3 technical replicates.

FIG. 6 shows the CDC screening results of human biparatopic anti-GPRC5D antibodies. MM.1R cells were incubated with bivalent biparatopic anti-GPRC5D antibodies in the presence of human serum for 24 hours and cell viability was determined using CellTiter-Glo®. Data represent mean±SEM, 3 technical replicates. Data represent mean±SEM, 3 technical replicates.

FIG. 7 illustrates the CDC screening results depicting the variants of human anti-GPRC5D antibodies derived from GP5B66 and GP5B82 clones. MM.1R cells were incubated with various bivalent and bivalent monospecific anti-GPRC5D antibodies with CDC-enhancing mutations (JAWA) and afucosylated (AFU) in the presence of human serum for 24 hours and cell viability was determined using CellTiter-Glo®. Data represent mean±SEM, 3 technical replicates. Data represent mean±SEM, 3 technical replicates.

FIG. 8 presents the ADCC screening results of human anti-GPRC5D antibodies. MM.1R cells were incubated with a set of 4 monovalent anti-GPRC5D antibodies and primary healthy donor peripheral NK cells at an effector to target ratio of 5:1 for 48 hours. Using flow cytometry, ADCC activity was compared to bivalent positive control antibody, GC5B1231 and negative control isotypes, B23B259 (bivalent) and B23B293.001 (monovalent). Data represent mean±SEM, n=2 NK donors . . .

FIG. 9 presents the ADCC screening results of rat anti-GPRC5D antibodies. MM.1R cells were incubated with monovalent rat anti-GPRC5D antibodies and primary healthy donor peripheral NK cells at an effector to target ratio of 5:1 for 48 hours, using flow cytometry with negative control isotypes, B23B259 (bivalent) and B23B293.001 (monovalent). Data represent mean±SEM, n=2 NK donors.

FIG. 10 shows the ADCC screening results of humanized anti-GPRC5D antibodies derived from the previous rat anti-GPRC5D antibodies. MM.1R cells were incubated with monovalent humanized anti-GPRC5D antibodies and primary healthy donor peripheral NK cells at an effector to target ratio of 5:1 for 48 hours, using flow cytometry with the positive control monovalent anti-GPRC5D antibody, GC5B1552 and negative control isotype, and B23B293 (monovalent). Data represent mean±SEM, n=2 NK donors . . .

FIG. 11 shows the ADCC screening results of human anti-GPRC5D antibody variants derived from the GP5B66 clone. MM.1R cells were incubated with monovalent humanized anti-GPRC5D antibodies and primary healthy donor peripheral NK cells at an effector to target ratio of 5:1 for 48 hours, using flow cytometry with isotype control antibodies B23B293 (monovalent) and B23B259 (bivalent). Data represent mean±SEM, n=2 NK donors . . .

FIG. 12 shows the ADCC screening results of human anti-GPRC5D antibody variants derived from the GP5B82 clone. MM.1R cells were incubated with monovalent (GC5B1552 and GC5B 1509) and bivalent (GC5B776 and GC5B768) humanized anti-GPRC5D antibodies and primary healthy donor peripheral NK cells at an effector to target ratio of 5:1 for 48 hours, using flow cytometry with isotype control antibodies B23B293 (monovalent) and B23B259 (bivalent). Data represent mean±SEM, n=2 NK donors . . .

FIG. 13A, FIG. 13B, FIG. 13C, and FIG. 13D depict the ADCP activity of anti-GPRC5D antibody clone GC5B 1552 against a panel of GPRC5D⁺ cell lines. GC5B1552 and isotype control (B23B293) were added at various concentrations (0.005 to 300 nM) with CFSE-labeled target cells and primary healthy donor MI-macrophages cells (healthy donor CD14⁺ human monocytes were differentiated for 6 days in rhM-CSF and then 1 day in rhIFN-γ; n=5 for H929 (FIG. 13A) and MM.1R (FIG. 13B); n=6 for H929 GPRC5D KO and H929 BCMA KO (FIG. 13D); 3:1 E:T ratio). After 2 hours, flow cytometry was used to identify CFSE-labeled target cells fluorescing in the FITC channel; macrophages were identified as CD11b⁺. Percent phagocytosis=([phagocytosed target cells]/[phagocytosed target cells+non-phagocytosed target cells]) x100. Mean+SEM is shown.

FIG. 14 shows the anti-tumor efficacy of GC5B1552.AFU.001 in MM.1S luc human MM xenografts in NK-92.CD16 cell humanized NSG mice. Disseminated MM. IS luc tumors were imaged for bioluminescence (BLI) twice weekly and the results presented as average radiance (p/s/cm2/sr)+SEM (n=9-10/group). Treatment with GC5B1552.AFU.001 or B23B293.001 control antibodies is represented by line underneath X-axis. * Denotes significant difference of GC5B1552.AFU.001-treated groups on Day 28 (n≥9/group) versus the control group.

FIG. 15 shows the anti-tumor efficacy of GC5B1552.AFU.001 in OPM-2 luc human MM xenografts in NK-92.CD16 cell humanized NSG mice. Disseminated OPM-2 luc tumors were imaged for bioluminescence (BLI) once weekly and the results presented as average radiance (p/s/cm2/sr)+SEM (n=7-10/group). Treatment with GC5B1552.AFU.001 or B23B293.001 control antibodies is represented by line underneath X-axis. * Denotes significant difference of GC5B1552.AFU.001-treatedgroups on Day 53 (n>7/group) versus the control group.

FIG. 16A, FIG. 16B, FIG. 16C, FIG. 16D, FIG. 16E, and FIG. 16F illustrate the kinetic cell binding of GPRC5D hits on H929 (FIG. 16A, FIG. 16C, and FIG. 16E)) or MM.1R (FIG. 16B, FIG. 16D, and FIG. 16F) cells for 1, 3, 5, and 24 hr at 37° C. at 50 nM, 5 nM and 0.5 nM.

FIG. 17 presents individual serum GC5B1552.AFU concentration-time profiles following a dingle IV dose of 0.5 mg/kg of GC5B 1552.AFU in cynomolgus monkeys (Group 1).

FIG. 18 presents individual serum GC5B1509.AFU concentration-time profiles following a single IV dose of 0.5 mg/kg of GC5B 1509.AFU in cynomolgus monkeys (Group 2).

FIG. 19 shows mean (SD) serum GC5B1231.AFU concentrations-time profiles following a single dose of GC5B1231.AFU in cynomolgus monkeys.

FIG. 20 presents individual serum GC5B1231.AFU concentrations-time profiles following a single IV dose of 1 mg/kg of GC5B1231.AFU in Cynomolgus Monkeys (Group 1).

FIG. 21 presents individual serum GC5B1231.AFU concentrations-time profiles following a single IV dose of 10 mg/kg of GC5B1231.AFU in cynomolgus monkeys (Group 2).

FIG. 22 presents individual serum GC5B1231.AFU concentrations-time profiles following a single SC dose of 10 mg/kg of GC5B1231.AFU in cynomolgus monkeys (Group 3).

### DETAILED DESCRIPTION

The present disclosure relates to antibodies or antigen-binding fragments that specifically bind to GPRC5D, a critical target for multiple myeloma treatment.

### Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. Sec, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F (ab) 2 fragments, F (ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules. The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

“Antigen-binding fragment” refers to a portion of the protein that binds an antigen. Antigen binding fragments may be synthetic, enzymatically obtainable or genetically engineered polypeptides and include portions of an immunoglobulin that bind an antigen, such as VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv), stapled single chain Fv (spFv), or diabody. Antigen binding fragments may also be conjugated to other antibodies, proteins, antigen-binding fragments or alternative scaffolds which may be monospecific or multispecific to engineer bispecific and multispecific proteins.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibodies or antigen-binding fragments described herein terms such as “specifically binding to,” and analogous terms are also used interchangeably herein and refer to antibodies or antigen-binding fragments that specifically bind to an antigen, such as a polypeptide. A antibodies or antigen-binding fragments that binds to or specifically binds to an antigen can be identified, for example, by immunoassays, Octet®, Biacore®, or other techniques known to those of skill in the art. In some embodiments, antibodies or antigen-binding fragments binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen-binding fragment to a “non-target” protein is less than about 10% of the binding of the antibody or antigen-binding fragment to its particular target antigen, for example, as determined by fluorescence activated cell sorting (FACS) analysis or RIA. An antibody or antigen-binding fragment that binds to an antigen includes one that is capable of binding the antigen with sufficient affinity such that the antibody or antigen-binding fragment is useful, for example, as a therapeutic and/or diagnostic agent in targeting the antigen. In certain embodiments, an antibody or antigen-binding fragment that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen-binding fragment binds to an epitope of an antigen that is conserved among the antigen from different species.

In certain embodiments, the antibodies or antigen-binding fragments can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the antibodies or antigen-binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1:755-68 (2006)). Also available for the preparation of human monoclonal antibodies are methods described in Cole et al., Monoclonal Antibodies and Cancer Therapy 77 (1985); Boerner et al., J. Immunol. 147 (1): 86-95 (1991); and van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6 (5): 561-66 (1995); Brüggemann and Taussing, Curr. Opin. Biotechnol. 8 (4): 455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the antibodies or antigen-binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for u and & isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th cd. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: a, 8, and γ contain approximately 450 amino acids, while u and & contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR | L24--L34 | L24--L34 | L26--L32 or | L30--L36 | L27-- |
| L1 | | | L24--L34 | | L38 |
| CDR | L50--L56 | L50--L56 | L50--L52 or | L46--L55 | L56-- |
| L2 | | | L50--L56 | | L65 |
| CDR | L89--L97 | L89--L97 | L91--L96 or | L89--L96 | L105- |
| L3 | | | L89--L97 | | L117 |
| CDR | H31--H35B | H26--H35B | H26--H32 | H30--H35B | H27-- |
| H1 | (Kabat | | . . . 34 | | H38 |
| | Numbering) | | | | |
| CDR | H31--H35 | H26--H35 | H26--H32 | H30--H35 | |
| H1 | (Chothia | | | | |
| | Numbering) | | | | |
| CDR | H50--H65 | H50--H58 | H53--H55 or | H47--H58 | H56-- |
| H2 | | | H52--H56 | | H65 |
| CDR | H95--H102 | H95--H102 | H96--H101 | H93--H101 | H105- |
| H3 | | | or | | H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include Clq binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-GPRC5D antibody may result from one or more (such as, for example, about 1 to about 25, about 1 to about 20, about 1 to about 15, about 1 to about 10, or about 1 to about 5) changes to an amino acid sequence of a native or previously unmodified anti-GPRC5D antibody. Variants may be naturally occurring, such as allelic or splice variants, or may be artificially constructed. Polypeptide variants may be prepared from the corresponding nucleic acid molecules encoding the variants. In specific embodiments, the anti-GPRC5D antibody variant at least retains anti-GPRC5D antibody functional activity, respectively. In specific embodiments, an anti-GPRC5D antibody variant binds GPRC5D. In certain embodiments, the variant is encoded by a single nucleotide polymorphism (SNP) variant of a nucleic acid molecule that encodes anti-GPRC5D antibody VH or VL regions or subregions, such as one or more CDRs.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

The meaning of “substantially the same” can differ depending on the context in which the term is used. Because of the natural sequence variation likely to exist among heavy and light chains and the genes encoding them, one would expect to find some level of variation within the amino acid sequences or the genes encoding the antibodies or antigen-binding fragments described herein, with little or no impact on their unique binding properties (e.g., specificity and affinity). Such an expectation is due in part to the degeneracy of the genetic code, as well as to the evolutionary success of conservative amino acid sequence variations, which do not appreciably alter the nature of the encoded protein. Accordingly, in the context of nucleic acid sequences, “substantially the same” means at least 65% identity between two or more sequences. Preferably, the term refers to at least 70% identity between two or more sequences, more preferably at least 75% identity, more preferably at least 80% identity, more preferably at least 85% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, and more preferably at least 99% or greater identity. The percent identity between two sequences is a function of the number of identical positions shared by the sequences (i.e., % homology= #of identical positions/total #of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

The degree of variation that may occur within the amino acid sequence of a protein without having a substantial effect on protein function is much lower than that of a nucleic acid sequence, since the same degeneracy principles do not apply to amino acid sequences.

Accordingly, in the context of an antibody or antigen-binding fragment, “substantially the same” means antibodies or antigen-binding fragments having 90%, 91%, 92%, 93%, 94%, 95%, 96%, 97%, 98%, or 99% identity to the antibodies or antigen-binding fragments described. Other embodiments include GPRC5D specific antibodies, or antigen-binding fragments, that have framework, scaffold, or other non-binding regions that do not share significant identity with the antibodies and antigen-binding fragments described herein, but do incorporate one or more CDRs or other sequences needed to confer binding that are 90%, 91%, 92%, 93%, 94%, 95%, 96%, 97%, 98%, or 99% identical to such sequences described herein. A “vector” is a replicon, such as plasmid, phage, cosmid, or virus in which another nucleic acid segment may be operably inserted so as to bring about the replication or expression of the segment.

The term “valent” as used herein denotes the presence of a specified number of binding sites in an antigen binding protein. A natural antibody for example or a full length antibody has two binding sites and is bivalent. As such, the terms “monovalent,” “trivalent,” “tetravalent,” “pentavalent” and “hexavalent” denote the presence of one binding site, two binding site, three binding sites, four binding sites, five binding sites, and six binding sites, respectively, in an an antibody)

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

Unless otherwise specified, a “polynucleotide sequence encoding an amino acid sequence” includes all nucleotide sequences that are degenerate versions of each other and that encode the same amino acid sequence. The phrase polynucleotide sequence that encodes a protein or an RNA may also include introns to the extent that the nucleotide sequence encoding the protein may in some version contain an intron(s).

The term “vector” refers to a substance that is used to carry or include a nucleic acid sequence, including for example, a nucleic acid sequence encoding an antibody or antigen-binding fragment as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopcia, European Pharmacopeia, or other generally recognized Pharmacopeia for use in animals, and more particularly in humans.

“Excipient” means a pharmaceutically acceptable material, composition, or vehicle, such as a liquid or solid filler, diluent, solvent, or encapsulating material. The term “excipient” can also refer to a diluent, adjuvant (e.g., Freunds' adjuvant (complete or incomplete) or vehicle.

In some embodiments, excipients are pharmaceutically acceptable excipients.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. Sec, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids.

The term “effective amount” or “therapeutically effective amount” as used herein refers to the amount of an antibody or a therapeutic molecule comprising an agent and the antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

As used herein, the terms “G-protein coupled receptor family C group 5 member D” and “GPRC5D” specifically include the human GPRC5D protein, for example as described in GenBank Accession No. BC069341, NCBI Reference Sequence: NP_061124.1 and UniProtKB/Swiss-Prot Accession No. Q9NZD1 (see also Brauner-Osborne, H. et al. 2001, Biochim. Biophys. Acta 1518, 237-248).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term

“comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### GPRC5D ANTIBODIES AND ANTIGEN-BINDING FRAGMENTS

Described herein are antibodies or antigen-binding fragments specifically binding GPRC5D. The general structure of an antibody molecule comprises an antigen binding domain, which includes heavy and light chains, and the Fc domain, which serves a variety of functions, including complement fixation and binding antibody receptors.

The described GPRC5D-specific antibodies or antigen-binding fragments include all isotypes, IgA, IgD, IgE, IgG and IgM, and synthetic multimers of the four-chain immunoglobulin structure. The described antibodies or antigen-binding fragments also include the IgY isotype generally found in hen or turkey serum and hen or turkey egg yolk.

The GPRC5D-specific antibodies and antigen-binding fragments may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to a human patient.

In some embodiments, the antibodies or antigen-binding fragments are chimeric. As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

In some embodiments, the antibodies are humanized antibodies. Humanized antibodies may be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin.

The antibodies or antigen-binding fragments described herein can occur in a variety of forms, but will include one or more of the antibody CDRs shown in Table 2.

TABLE 2
List of CDR Regions for a representative GPRC5D antibody

| Numbering | | CDR1 | CDR2 | CDR3 |
|---|---|---|---|---|
| Format | Chain | (SEQ ID NO) | (SEQ ID NO) | (SEQ ID NO) |
| AbM | Heavy | GFTFSNYGMH (6) | FIWYDGSNKY (7) | ITGYSTRWGDY (8) |
| | Light | RASQGIRNDLG (9) | AASSLQS (10) | LQRNTYPLT (11) |
| Kabat | Heavy | NYGMH (12) | FIWYDGSNKYYADSVKG (13) | ITGYSTRWGDY (8) |
| | Light | RASQGIRNDLG (9) | AASSLQS (10) | LQRNTYPLT (11) |
| Chothia | Heavy | GFTFSNY (14) | WYDGSN (15) | ITGYSTRWGDY (8) |
| | Light | RASQGIRNDLG (9) | AASSLQS (10) | LQRNTYPLT (11) |
| IMGT | Heavy | GFTFSNYG (16) | IWYDGSNK (17) | APITGYSTRWGDY (18) |
| | Light | QGIRND (19) | AAS | LQRNTYPLT (11) |
| Contact | Heavy | SNYGMH (21) | WVAFIWYDGSNKY (22) | APITGYSTRWGD (23) |
| | Light | RNDLGWY (24) | RLIYAASSLQ (25) | LQRNTYPL (26) |

Described herein are antibodies and antigen-binding fragments specifically binding to GPRC5D. In some embodiments, the GPRC5D-specific antibodies or antigen-binding fragments are human, humanized IgG, or derivatives thereof. While the GPRC5D-specific antibodies or antigen-binding fragments exemplified herein are human or humanized, the antibodies or antigen-binding fragments exemplified may be chimerized.

In some embodiments are provided a GPRC5D-specific antibody, or an antigen-binding fragment thereof, comprising a heavy chain comprising a CDR1, a CDR2, and a CDR3 of any one of the antibodies described in Table 2. In some embodiments are provided a GPRC5D-specific antibody, or an antigen-binding fragment thereof, comprising a heavy chain comprising a CDR1, a CDR2, and a CDR3 of any one of the antibodies described in Table 2 and a light chain comprising a CDR1, a CDR2, and a CDR3 of any one of the antibodies described in Table 2.

In some embodiments, the GPRC5D-specific antibodies and antigen-binding fragments comprise a heavy chain complementarity determining region 1 (CDR1), a heavy chain complementarity determining region 2 (CDR2), and a heavy chain complementarity determining region 3 (CDR3) comprising the amino acid sequences of SEQ ID NO: 6, 7, and 8, respectively, and a light chain complementarity determining region 1 (CDR1), a light chain complementarity determining region 2 (CDR2), and a light chain complementarity determining region 3 (LCDR3) comprising the amino acid sequences of SEQ ID NO: 9, 10, and 11, respectively. This GPRC5D -specific antibody or antigen-binding fragment may comprise human framework sequences. In some embodiments, the GPRC5D-specific antibodies and antigen-binding fragments comprise a heavy chain variable domain substantially the same as, or identical to, SEQ ID NO: 1 and a light chain variable domain substantially the same as, or identical to, SEQ ID NO: 2. In some embodiments, the GPRC5D-specific antibodies and antigen-binding fragments comprise a heavy chain substantially the same as, or identical to, SEQ ID NO: 3 and a light chain variable domain substantially the same as, or identical to, SEQ ID NO: 5. The CDRs, the heavy chain variable domain, light chain variable domain, the heavy chain, and the light chain of antibodies discussed in this paragraph are suitable for inclusion in multispecific constructs in which one arm is an anti-GPRC5D arm.

In some embodiments, the GPRC5D-specific antibodies and antigen-binding fragments comprise a heavy chain complementarity determining region 1 (CDR1), a heavy chain complementarity determining region 2 (CDR2), and a heavy chain complementarity determining region 3 (CDR3) comprising the amino acid sequences of SEQ ID NO: 12, 13, and 8, respectively, and a light chain complementarity determining region 1 (CDR1), a light chain complementarity determining region 2 (CDR2), and a light chain complementarity determining region 3 (LCDR3) comprising the amino acid sequences of SEQ ID NO: 9, 10, and 11, respectively. This GPRC5D-specific antibody or antigen-binding fragment may comprise human framework sequences. In some embodiments, the GPRC5D-specific antibodies and antigen-binding fragments comprise a heavy chain variable domain substantially the same as, or identical to, SEQ ID NO: 1 and a light chain variable domain substantially the same as, or identical to, SEQ ID NO: 2. In some embodiments, the GPRC5D-specific antibodies and antigen-binding fragments comprise a heavy chain substantially the same as, or identical to, SEQ ID NO: 3 and a light chain variable domain substantially the same as, or identical to, SEQ ID NO: 5. The CDRs, the heavy chain variable domain, light chain variable domain, the heavy chain, and the light chain of antibodies discussed in this paragraph are suitable for inclusion in multispecific constructs in which one arm is an anti-GPRC5D arm.

In some embodiments, the GPRC5D-specific antibodies and antigen-binding fragments comprise a heavy chain complementarity determining region 1 (CDR1), a heavy chain complementarity determining region 2 (CDR2), and a heavy chain complementarity determining region 3 (CDR3) comprising the amino acid sequences of SEQ ID NO: 14, 15, and 8, respectively, and a light chain complementarity determining region 1 (CDR1), a light chain complementarity determining region 2 (CDR2), and a light chain complementarity determining region 3 (LCDR3) comprising the amino acid sequences of SEQ ID NO: 9, 10, and 11, respectively. This GPRC5D-specific antibody or antigen-binding fragment may comprise human framework sequences. In some embodiments, the GPRC5D-specific antibodies and antigen-binding fragments comprise a heavy chain variable domain substantially the same as, or identical to, SEQ ID NO: 1 and a light chain variable domain substantially the same as, or identical to, SEQ ID NO: 2. In some embodiments, the GPRC5D-specific antibodies and antigen-binding fragments comprise a heavy chain substantially the same as, or identical to, SEQ ID NO: 3 and a light chain variable domain substantially the same as, or identical to, SEQ ID NO: 5. The CDRs, the heavy chain variable domain, light chain variable domain, the heavy chain, and the light chain of antibodies discussed in this paragraph are suitable for inclusion in multispecific constructs in which one arm is an anti-GPRC5D arm.

In some embodiments, the GPRC5D-specific antibodies and antigen-binding fragments comprise a heavy chain complementarity determining region 1 (CDR1), a heavy chain complementarity determining region 2 (CDR2), and a heavy chain complementarity determining region 3 (CDR3) comprising the amino acid sequences of SEQ ID NO: 16, 17, and 18, respectively, and a light chain complementarity determining region 1 (CDR1), a light chain complementarity determining region 2 (CDR2), and a light chain complementarity determining region 3 (LCDR3) comprising the amino acid sequences of SEQ ID NO: 19, 20, and 11, respectively. This GPRC5D-specific antibody or antigen-binding fragment may comprise human framework sequences. In some embodiments, the GPRC5D-specific antibodies and antigen-binding fragments comprise a heavy chain variable domain substantially the same as, or identical to, SEQ ID NO: 1 and a light chain variable domain substantially the same as, or identical to, SEQ ID NO: 2. In some embodiments, the GPRC5D-specific antibodies and antigen-binding fragments comprise a heavy chain substantially the same as, or identical to, SEQ ID NO: 3 and a light chain variable domain substantially the same as, or identical to, SEQ ID NO: 5. The CDRs, the heavy chain variable domain, light chain variable domain, the heavy chain, and the light chain of antibodies discussed in this paragraph are suitable for inclusion in multispecific constructs in which one arm is an anti-GPRC5D arm.

In some embodiments, the GPRC5D-specific antibodies and antigen-binding fragments comprise a heavy chain complementarity determining region 1 (CDR1), a heavy chain complementarity determining region 2 (CDR2), and a heavy chain complementarity determining region 3 (CDR3) comprising the amino acid sequences of SEQ ID NO: 21, 22, and 23, respectively, and a light chain complementarity determining region 1 (CDR1), a light chain complementarity determining region 2 (CDR2), and a light chain complementarity determining region 3 (LCDR3) comprising the amino acid sequences of SEQ ID NO: 24, 25, and 26, respectively. This GPRC5D-specific antibody or antigen-binding fragment may comprise human framework sequences. In some embodiments, the GPRC5D-specific antibodies and antigen-binding fragments comprise a heavy chain variable domain substantially the same as, or identical to, SEQ ID NO: 1 and a light chain variable domain substantially the same as, or identical to, SEQ ID NO: 2. In some embodiments, the GPRC5D-specific antibodies and antigen-binding fragments comprise a heavy chain substantially the same as, or identical to, SEQ ID NO: 3 and a light chain variable domain substantially the same as, or identical to, SEQ ID NO: 5. The CDRs, the heavy chain variable domain, light chain variable domain, the heavy chain, and the light chain of antibodies discussed in this paragraph are suitable for inclusion in multispecific constructs in which one arm is an anti-GPRC5D arm.

In some embodiments, the antibodies or antigen-binding fragments are IgG, or derivatives thereof, e.g., IgG1, IgG2, IgG3, and IgG4 isotypes. In some embodiments wherein the antibody is of IgG1 isotype, the antibody comprises an IgG1 Fc region.

### Humanized GPRC5D Antibodies

The antibodies described herein include humanized antibodies. Humanized antibodies, such as the humanized antibodies disclosed herein can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology 28 (4/5): 489-498 (1991); Studnicka et al., Protein Engineering 7 (6): 805-814 (1994); and Roguska et al., PNAS 91:969-973 (1994)), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13 (5): 353-60 (2000), Morea et al., Methods 20 (3): 267 79 (2000), Baca et al., J. Biol. Chem. 272 (16): 10678-84 (1997), Roguska et al., Protein Eng. 9 (10): 895 904 (1996), Couto et al., Cancer Res. 55 (23 Supp): 5973s-5977s (1995), Couto et al., Cancer Res. 55 (8): 1717-22 (1995), Sandhu JS, Gene 150 (2): 409-10 (1994), and Pedersen et al., J. Mol. Biol. 235 (3): 959-73 (1994). See also U.S. Patent Pub. No. US 2005/0042664 A1 (Feb. 24, 2005), each of which is incorporated by reference herein in its entirety.

In some embodiments, antibodies provided herein can be humanized antibodies that bind to GPRC5D, including human GPRC5D. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., Nature 321:522-25 (1986); Riechmann et al., Nature 332:323-27 (1988); and Verhocyen et al., Science 239:1534-36 (1988)), by substituting hypervariable region sequences for the corresponding sequences of a human antibody.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., FASEB J. 9:133-39 (1995)). In the technique of SDR grafting, only the SDR residues are grafted onto the human antibody framework (see, e.g., Kashmiri et al., Methods 36:25-34 (2005)).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., J. Immunol. 151:2296-308 (1993); and Chothia et al., J. Mol. Biol. 196:901-17 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285-89 (1992); and Presta et al., J. Immunol. 151:2623-32 (1993)). In some cases, the framework is derived from the consensus sequences of the most abundant human subclasses, V1.6 subgroup I (V1.6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-25 (2002)).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-24 (2002)), Modeller (Sali and Blundell, J. Mol. Biol. 234:779-815 (1993)), and Swiss PDB Viewer (Guex and Peitsch, Electrophoresis 18:2714-23 (1997)). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol. 44:1986-98 (2007)).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol. 23:1105-16 (2005); Dufner et al., Trends Biotechnol. 24:523-29 (2006); Feldhaus et al., Nat. Biotechnol. 21:163-70 (2003); and Schlapschy et al., Protein Eng. Des. Sel. 17:847-60 (2004)).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol. 224:487-99 (1992)), or from the more limited set of target residues identified by Baca et al. J. Biol. Chem. 272:10678-84 (1997).

In FR shuffling, whole FRs are combined with the non-human CDRs instead of creating combinatorial libraries of selected residue variants (see, e.g., Dall'Acqua et al., Methods 36:43-60 (2005)). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., Mol. Immunol. 44:3049-60 (2007)).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering 7:805-14 (1994); U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 525:405-23 (2009), xiv, and De Groot et al., Cell. Immunol. 244:148-153 (2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

### Mutations, Deletions, or Insertions

Variations may be a mutation, deletion, or insertion of one or more codons encoding the antibody or polypeptide that results in a change in the amino acid sequence as compared with the original antibody or polypeptide. Sites of interest for substitutional mutagenesis include the CDRs and FRs.

Amino acid mutations can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid mutations. Insertions or deletions may optionally be in the range of about 1 to 5 amino acids. In certain embodiments, the mutation, deletion, or insertion includes fewer than 25 amino acid mutations, fewer than 20 amino acid mutations, fewer than 15 amino acid mutations, fewer than 10 amino acid mutations, fewer than 5 amino acid mutations, fewer than 4 amino acid mutations, fewer than 3 amino acid mutations, or fewer than 2 amino acid mutations relative to the original molecule. In a specific embodiment, the mutation is a conservative amino acid mutation made at one or more predicted non-essential amino acid residues. The variation allowed may be determined by systematically making insertions, deletions, or mutations of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid mutation, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary mutations are shown in Table 3 below.

TABLE 3
Amino Acid Mutations

| Original | Exemplary |
|---|---|
| Residue | Mutations |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; |
| | Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative mutations will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., mutations) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided in the section below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant antibody sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

### F_{C} MUTATIONS WITH ENHANCED EFFECTOR FUNCTIONS

The GPRC5D antibodies provided herein have mutations at the lysine at position 248 (K248) (EU numbering) and the threonine at position 437 (T437) (EU numbering) in the Fc region. Lysine at position 248 (K248) (EU numbering) and threonine at position 437 (T437) (EU numbering) are both conserved residues in the Fc regions among different IgG subtypes (Zhang, D., et al., supra). Fc mutations, T437R and K248E (EU numbering), were shown to facilitate oligomerization of antibodies upon binding antigens at the cell surface, and possess enhanced effector functions (Zhang, D., et al., supra). T437R and K248E double mutations (“RE mutations”) were shown to confer CDC activity on wildtype IgG1 antibodies that did not possess CDC activity in a dose-dependent manner (Zhang, D., et al., supra; PCT/US21/27666, PCT/US22/78351, and PCT/US22/78355).

The “EU numbering” or “EU index” is generally used when referring to a residue in an immunoglobulin heavy chain constant region. It refers to the residue numbering of the human IgG1 EU antibody. It is computed by alignment of an antibody sequence with the Eu antibody sequence (Edelman, G. M., et al., Proc Natl Acad Sci USA, 1969, 63 (1): 78-85; Kabat, et al., supra), so that each residue that is homologous to a residue in the EU antibody will have the same residue number as that EU residue.

### Antibody-Dependent Cell-Mediated Cytotoxicity (ADCC), Complement-Dependent Cytotoxicity (CDC), and Antibody-Dependent Cellular Phagocytosis Effector Functions

The GPRC5D antibodies provided herein have enhanced ADCC activity and enhanced CDC activity.

Therapeutic antibodies bind Fc receptors on the cell surface of effector cells, such as natural killer (NK) cells, macrophages, mononuclear phagocytes, neutrophils and eosinophils (Saunder, K. O., Front Immunol., 2019, 10:1296), giving rise to important antibody-dependent effector functions, such as antibody-dependent cell-mediated cytotoxicity (ADCC) and antibody-dependent cell-mediated phagocytosis (ADCP). A family of receptors for IgG Fc regions was referred to as the Fcγ receptors (FcγRs) (Cohen-Solal, J. F., Immunol Lett., 2004, 92 (3): 199-205), and is comprised of FcγRI; FcγRII, including isoforms FcγRIIa, FcγRIIb, and FcγRIIc; and FcγRIII, including isoforms FcγRIIIa and FcγRIIIb (Jefferis, R. and Lund, J., Immunol Lett., 2002, 82 (1-2): 57-65).

Among various effector functions, ADCC and ADCP have been shown to possess clinically significant anti-tumor efficacy. For example, ADCC was shown to be an important mechanism for the anti-tumor efficacy of trastuzumab in vitro, as evidenced by NK cells' capability to kill trastuzumab-coated tumor cells via a FcγRIII receptor (CD16)-mediated ADCC mechanism (Cooley, S., et al., Exp Hematol., 1999; 27 (10): 1533-41; Carson, W. E., et al., Eur J Immunol., 2001, 31 (10): 3016-3025; Kubo, M., et al., Anticancer Res., 2003, 23 (6a): 4443-9) and in vivo, as evidenced by increased numbers of NK cells in tumor infiltrates after trastuzumab treatment (Clynes, R. A., et al., Nat Med., 2000, 6 (4): 443-6; Arnould, L., et al., Br J Cancer, 2006, 94 (2): 259-67). Additionally, macrophage-mediated ADCP has been shown to be important in the anti-tumor efficacy of trastuzumab (Shi, Y., et al., J Immunol., 2015, 194 (9): 4379-86).

Antibodies with no or low fucosylation have shown dramatically enhanced ADCC activity due to the enhancement in their binding capacity to FcγRIIIa binding without any detectable change in CDC or antigen binding capability (Okazaki, A., et al., J Mol Biol., 2004, 336 (5): 1239-49; Kanda, Y., et al., Glycobiology, 2007, 17 (1): 104-18). N-oligosaccharides of antibody Fc regions are essential for binding to FcγR, which engages antibody effector functions (Yamane-Ohnuki, N. and Satoh, M., Mabs, 2009, 1 (3): 230-6).

The absence of fucose on N-oligosaccharides of antibody Fc regions have been shown to dramatically enhance antibodies' binding capacity to FcγRIIIa receptors present on immune effector cells such as natural killer (NK) cells and macrophages, giving rise to anti-tumor therapeutic effect (Pereira, N. A., et al., supra). The FcγRIIIa receptors bind Fc regions via interactions with the hinge region and the CH2 domain of the Fc (Radacv, S., et al., J Biol Chem., 2001, 276:16469-77; Sondermann, P., et al., Nature, 2000, 406:267-73). The absence of fucose thus eliminates the steric hindrance and enhances the Fc-FcγRIIIa interaction, leading to enhanced effector functions (Pereira, N. A., et al., supra).

Complement-dependent cytotoxicity (CDC) is another important antibody effector function. In the antibody-dependent classical complement activation pathway, binding between the complement Clq heterohexameric headpiece and an oligomeric antibody complex initiates the proteolytic complement cascade (Wang, G. et al., Mol Cell, 2016, 63:135-45; Diebolder, C. A. et al., Science, 2014, 343:1260-3), which leads to the opsonization of target cells by C3-derived opsonins (e.g., C3b) and generation of potent inflammation mediators (C3a and C5a), ultimately resulting in the formation of membrane attack complex (MAC), C5b-C9, on the target cell membrane (Reis, E. S., et al., Nat Rev Immunol., 2018, 18:5-18). CDC has also been shown to possess clinically significant anti-tumor efficacy, e.g., in the anti-CD20 mAb rituximab and anti-CD38 mAb daratumumab (de Weers, M., et al., J Immunol., 2011, 186:1840-8; Lokhorst, H. M., et al., N Engl J Med., 2015, 373:1207-19; Taylor, R. P. and Lindorfer, M. A., Semin Immunol., 2016, 28:309-16).

Mutations in the Fc region that facilitate antibody oligomerization, such as the RE mutations (EU numbering), have been demonstrated to significantly enhance antibody CDC activity (Diebolder, C. A. et al., supra; Zhang, D., et al., supra; PCT/US21/27666, PCT/US22/78351, and PCT/US22/78355).

In some embodiments, the ADCC activity of the present GPRC5D antibodies is 10% higher than GPRC5D antibodies with normal fucosylation. In some embodiments, the ADCC activity of the present GPRC5D antibodies is 20% higher than GPRC5D antibodies with normal fucosylation. In some embodiments, the ADCC activity of the present GPRC5D antibodies is 30% higher than GPRC5D antibodies with normal fucosylation. In some embodiments, the ADCC activity of the present GPRC5D antibodies is 40% higher than GPRC5D antibodies with normal fucosylation. In some embodiments, the ADCC activity of the present GPRC5D antibodies is 50% higher than GPRC5D antibodies with normal fucosylation. In some embodiments, the ADCC activity of the present GPRC5D antibodies is 60% higher than GPRC5D antibodies with normal fucosylation. In some embodiments, the ADCC activity of the present GPRC5D antibodies is 70% higher than GPRC5D antibodies with normal fucosylation. In some embodiments, the ADCC activity of the present GPRC5D antibodies is 80% higher than GPRC5D antibodies with normal fucosylation. In some embodiments, the ADCC activity of the present GPRC5D antibodies is 90% higher than GPRC5D antibodies with normal fucosylation. In some embodiments, the ADCC activity of the present GPRC5D antibodies is more than 2 fold of that of GPRC5D antibodies with normal fucosylation. In some embodiments, the ADCC activity of the present GPRC5D antibodies is more than 3 fold of that of GPRC5D antibodies with normal fucosylation. In some embodiments, the ADCC activity of the present GPRC5D antibodies is more than 4 fold of that of GPRC5D antibodies with normal fucosylation. In some embodiments, the ADCC activity of the present GPRC5D antibodies is more than 5 fold of that of GPRC5D antibodies with normal fucosylation. In some embodiments, the ADCC activity of the present GPRC5D antibodies is more than 6 fold of that of GPRC5D antibodies with normal fucosylation. In some embodiments, the ADCC activity of the present GPRC5D antibodies is more than 7 fold of that of GPRC5D antibodies with normal fucosylation. In some embodiments, the ADCC activity of the present GPRC5D antibodies is more than 8 fold of that of GPRC5D antibodies with normal fucosylation. In some embodiments, the ADCC activity of the present GPRC5D antibodies is more than 9 fold of that of GPRC5D antibodies with normal fucosylation. In some embodiments, the ADCC activity of the present GPRC5D antibodies is more than 10 fold of that of GPRC5D antibodies with normal fucosylation.

In some embodiments, the antibodies described above also have higher CDC activities. In some embodiments, the CDC activity of the present GPRC5D antibodies is 10% higher than GPRC5D antibodies without RE mutations. In some embodiments, the CDC activity of the present GPRC5D antibodies is 20% higher than antibodies without RE mutations. In some embodiments, the CDC activity of the present GPRC5D antibodies is 30% higher than GPRC5D antibodies without RE mutations. In some embodiments, the CDC activity of the present GPRC5D antibodies is 40% higher than GPRC5D antibodies without RE mutations. In some embodiments, the CDC activity of the present GPRC5D antibodies is 50% higher than GPRC5D antibodies without RE mutations. In some embodiments, the CDC activity of the present GPRC5D antibodies is 60% higher than GPRC5D antibodies without RE mutations. In some embodiments, the CDC activity of the present GPRC5D antibodies is 70% higher than GPRC5D antibodies without RE mutations. In some embodiments, the CDC activity of the present GPRC5D antibodies is 80% higher than GPRC5D antibodies without RE mutations. In some embodiments, the CDC activity of the present GPRC5D antibodies is 90% higher than GPRC5D antibodies without RE mutations. In some embodiments, the CDC activity of the present GPRC5D antibodies is more than 2 fold of that of GPRC5D antibodies without RE mutations. In some embodiments, the CDC activity of the present GPRC5D antibodies is more than 3 fold of that of antibodies without RE mutations. In some embodiments, the CDC activity of the present GPRC5D antibodies is more than 4 fold of that of GPRC5D antibodies without RE mutations. In some embodiments, the CDC activity of the present GPRC5D antibodies is more than 5 fold of that of GPRC5D antibodies without RE mutations. In some embodiments, the CDC activity of the present GPRC5D antibodies is more than 6 fold of that of GPRC5D antibodies without RE mutations. In some embodiments, the CDC activity of the present GPRC5D antibodies is more than 7 fold of that of GPRC5D antibodies without RE mutations. In some embodiments, the CDC activity of the present GPRC5D antibodies is more than 8 fold of that of GPRC5D antibodies without RE mutations. In some embodiments, the CDC activity of the present GPRC5D antibodies is more than 9 fold of that of GPRC5D antibodies without RE mutations. In some embodiments, the CDC activity of the present GPRC5D antibodies is more than 10 fold of that of GPRC5D antibodies without RE mutations.

### Knob-into-Hole Mutations

GPRC5D antibodies disclosed herein may comprise different Fc moieties, thus the two subunits of the Fc domain are typically comprised in two non-identical polypeptide chains. Recombinant co-expression of these polypeptides and subsequent dimerization leads to several possible combinations of the two polypeptides. To improve the yield and purity of bispecific antibodies in recombinant production, it will thus be advantageous to introduce in the Fc domain of the GPRC5D antibody a modification promoting the association of the desired polypeptides.

Accordingly, in some embodiments, the Fc domain of the GPRC5D antibodies disclosed herein comprise a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain to enforce heterodimerization, which are well described e.g., in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with the heavy-light chain modifications (e.g., VH and VL exchange/replacement in one binding arm and the introduction of substitutions of charged amino acids with opposite charges in the CH1/CL interface) in the heretodimeric antibody which reduce heavy/light chain mispairing and Bence Jones-type side products.

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, in the CH3 domain of the first subunit of the Fc domain of the GPRC5D antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine I, phenylalanine (F), tyrosine (Y), and tryptophan (W).

Preferably said amino acid residue having a smaller side chain volume is selected from the group consisting of alanine (A), serine(S), threonine (T), and valine (V).

The protuberance and cavity can be made by altering the nucleic acid encoding the polypeptides, e.g., by site-specific mutagenesis, or by peptide synthesis.

In a specific embodiment, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In some embodiments, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In some embodiments, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In some embodiments, the heterodimerization approach described in EP1870459, is used alternatively. This approach is based on the introduction of charged amino acids with opposite charges at specific amino acid positions in the CH3/CH3 domain interface between the two subunits of the Fc domain. One preferred embodiment for the bispecific antibody of the invention are amino acid mutations R409D; K370E in one of the two CH3 domains (of the Fc domain) and amino acid mutations D399K; E357K in the other one of the CH3 domains of the Fc domain (numbering according to Kabat EU index).

In some embodiments, the GPRC5D antibodies of the present disclosure comprise amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In some embodiments, the GPRC5D antibodies of the present disclosure comprise amino acid mutations S354C, T366W in the CH3 domain of the first subunit of the Fc domain and amino acid mutations Y349C, T366S, L368A, Y407V in the CH3 domain of the second subunit of the Fc domain, or said bispecific antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In some embodiments, the heterodimerization approach described in WO 2013/157953 is used alternatively. In some embodiments, a first CH3 domain comprises amino acid mutation T366K and a second CH3 domain comprises amino acid mutation L351D (numberings according to Kabat EU index). In a further embodiment, the first CH3 domain comprises further amino acid mutation L351K. In a further embodiment, the second CH3 domain comprises further an amino acid mutation selected from Y349E, Y349D and L368E (preferably L368E) (numberings according to Kabat EU index).

In some embodiments, the heterodimerization approach described in WO 2012/058768 is used alternatively. In one embodiment a first CH3 domain comprises amino acid mutations L351Y, Y407A and a second CH3 domain comprises amino acid mutations T366A, K409F. In a further embodiment the second CH3 domain comprises a further amino acid mutation at position T411, D399, 5400, F405, N390, or K392, e.g. selected from a) T41IN, T41IR, T411Q, T411K, T411D, T411E or T411W, b) D399R, D399W, D399Y or D399K, c) S400E, 5400D, 5400R, or 5400K, d) F4051, F405M, F405T, F4055, F405V or F405W, c) N390R, N390K or N390D, f) K392V, K392M, K392R, K392L, K392F or K392E (numberings according to Kabat EU index). In a further embodiment a first CH3 domain comprises amino acid mutations L351Y, Y407A and a second CH3 domain comprises amino acid mutations T366V, K409F. In a further embodiment, a first CH3 domain comprises amino acid mutation Y407A and a second CH3 domain comprises amino acid mutations T366A, K409F. In a further embodiment, the second CH3 domain further comprises amino acid mutations K392E, T411E, D399R and 5400R (numberings according to Kabat EU index).

In some embodiments, the heterodimerization approach described in WO 2011/143545 is used alternatively, e.g., with the amino acid modification at a position selected from the group consisting of 368 and 409 (numbering according to Kabat EU index).

In some embodiments, the heterodimerization approach described in WO 2011/090762, which also uses the knobs-into-holes technology described above, is used alternatively. In one embodiment a first CH3 domain comprises amino acid mutation T366W and a second CH3 domain comprises amino acid mutation Y407A. In some embodiments, a first CH3 domain comprises amino acid mutation T366V and a second CH3 domain comprises amino acid mutation Y407T (numberings according to Kabat EU index).

In some embodiments, the GPRC5D antibody or its Fc domain is of IgG2 subclass and the heterodimerization approach described in WO 2010/129304 is used alternatively.

In some embodiments, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g. glutamic acid I, or aspartic acid (D), preferably K392D or N392D) and a second CH3 domain comprises amino acid substitution of D399, E356, D356, or E357 with a positively charged amino acid (e.g. lysine (K) or arginine I, preferably D399K, E356K, D356K, or E357K, and more preferably D399K and E356K). In a further embodiment, the first CH3 domain further comprises amino acid substitution of K409 or R409 with a negatively charged amino acid (e.g., glutamic acid I, or aspartic acid (D), preferably K409D or R409D). In a further embodiment the first CH3 domain further or alternatively comprises amino acid substitution of K439 and/or K370 with a negatively charged amino acid (e.g., glutamic acid I, or aspartic acid (D)) (all numberings according to Kabat EU index).

In some embodiments, the heterodimerization approach described in WO 2007/147901 is used alternatively. In some embodiments, a first CH3 domain comprises amino acid mutations K253E, D282K, and K322D and a second CH3 domain comprises amino acid mutations D239K, E240K, and K292D (numberings according to Kabat EU index).

In some embodiments, the heterodimerization approach described in WO 2007/110205 can be used alternatively.

In some embodiments, the first subunit of the Fc domain comprises amino acid substitutions K392D and K409D, and the second subunit of the Fc domain comprises amino acid substitutions D356K and D399K (numbering according to Kabat EU index).

### Monovalent Antibodies

In one aspect, provided herein are monovalent antibodies or fragments thereof specifically binding GPRC5D that carry RE mutations in the Fc region of the monovalent antibodies. As demonstrated in the EXAMPLES section, the combination of the monovalency with the RE mutations further enhances the CDC activity as compared with the RE mutations alone, among other advantages.

CDC activity requires the activation of the complement cascade, which in turn requires activation of complement component Clq by a hexamer of Fc regions co-planar to the surface of the target cell. This in turn requires concurrent binding of five or six antibody molecules in close proximity on the cell surface. Due to the requirement for Fc-mediated antibody oligomerization, CDC activity requires relatively high target receptor densities. With natural IgG antibodies, the stoichiometry of antibody-to-receptor binding can vary from 1:2 to 1:1 depending on antibody concentration, with the former highly favored due to avidity.

To maximize the number of antibodies that can bind a given number of cell surface receptors, the antibodies provided herein are formatted as monovalent antibodies to force 1:1 antibody-to-receptor binding stoichiometry. This allows more Fc regions to be brought to the surface of the cell with a given number of receptors compared to the natural bivalent IgG. The monovalent antibodies provided herein have more potent CDC activity than their natural bivalent counterparts.

Standard techniques known to those of skill in the art can be used to format an antibody as monovalent antibody, including, for example, in vitro expression of recombinant proteins.

Any monovalent antibody format may be applied in the present constructs as long as the format confers further enhanced CDC activity via increased oligomerization (e.g., hexamerization) of the binding molecules at a cell surface and/or increased Clq engagement.

In some embodiments, the monovalent GPRC5D antibody comprises one antigen-binding arm of no known specificity and another antigen-binding arm specific for GPRC5D.

In some embodiments, the monovalent GPRC5D antibody comprises an antigen-binding arm specific for GPRC5D, one antigen-binding arm of no known specificity or left without one binding arm.

In some embodiments, the GPRC5D binding domain of the monovalent GPRC5D antibody comprises a VH region and/or a VL region. In some embodiments, the GPRC5D binding domain of the monovalent antibody comprises a Fab fragment. In some embodiment, the GPRC5D binding domain of the monovalent GPRC5D antibody comprises a scFv. In some embodiments, the GPRC5D binding domain of the monovalent antibody comprises a single VH domain. In some embodiment, the GPRC5D binding domain of the monovalent GPRC5D antibody comprises a single VL domain. In some embodiment, the GPRC5D binding domain of the monovalent GPRC5D antibody comprises a protein domain specific for GPRC5D.

In some embodiments, the monovalent GPRC5D antibody comprises a Fab and a Fc domain, wherein the monovalent GPRC5D antibody comprises a first heavy chain (HCl), a second heavy chain (HC2) and a second light chain (LC2), wherein the HCl and HC2 form the Fc domain, and the HC2 and the LC2 form the Fab.

In some embodiments, the monovalent GPRC5D antibody, or antigen-binding fragment thereof, comprises a first heavy chain (HCl) comprising an amino acid sequence that is substantially the same as, or identical to, SEQ ID NO: 3. In some embodiments, the heavy chain comprises SEQ ID NO: 3. In some embodiments, the heavy chain consists of SEQ ID NO: 3.

In some embodiments, the monovalent GPRC5D antibody, or antigen-binding fragment thereof, comprises a second heavy chain (HC2) comprising an amino acid sequence that is substantially the same as, or identical to, SEQ ID NO: 4. In some embodiments, the heavy chain comprises SEQ ID NO: 4. In some embodiments, the heavy chain consists of SEQ ID NO: 4.

In some embodiments, the monovalent GPRC5D antibody, or antigen-binding fragment thereof, comprises a second light chain (LC2) comprising an amino acid sequence that is substantially the same as, or identical to, SEQ ID NO: 5. In some embodiments, the heavy chain comprises SEQ ID NO: 5. In some embodiments, the heavy chain consists of SEQ ID NO: 5.

In some embodiments, the monovalent GPRC5D antibody, comprises a first heavy chain comprising SEQ ID NO: 3, a second heavy chain comprising SEQ ID NO: 4 and a second light chain comprising SEQ ID NO: 5.

In some embodiments, the monovalent GPRC5D antibody comprises a scFv and a Fc domain, wherein the monovalent GPRC5D antibody comprises two polypeptides; wherein the first polypeptide comprises the scFv at the N-terminus and a domain at the C-terminus that forms the Fc domain with the second polypeptides.

In some embodiments, the monovalent GPRC5D antibody comprises a single VH domain and a Fc domain, wherein the monovalent GPRC5D antibody comprises two polypeptides;

wherein the first polypeptide comprises the single VH domain at the N-terminus and a domain at the C-terminus that forms the Fc domain with the second polypeptides.

In some embodiments, the monovalent GPRC5D antibody comprises a single VL domain and a Fc domain, wherein the monovalent GPRC5D antibody comprises two polypeptides;

wherein the first polypeptide comprises the single VL domain at the N-terminus and a domain at the C-terminus that forms the Fc domain with the second polypeptides.

In some embodiment, the monovalent GPRC5D antibody comprises a protein domain specific for GPRC5D and a Fc domain, wherein the monovalent antibody comprises two polypeptides; wherein the first polypeptide comprises the protein domain specific for GPRC5D at the N-terminus and a domain at the C-terminus that forms the Fc domain with the second polypeptides.

Afucosylation

Antibody glycosylation is a type of posttranslational modification that may occur via the addition of oligosaccharides to antibodies through two types of covalent linkages: linkages on asparagine residues (N-oligosaccharides) s or on serine/threonine residues (O-oligosaccharides) (Alter, G., et al., Semin Immunol., 2018, 39:102-10), and profoundly affect therapeutic functions of antibodies (Walsh, G. and Jefferis, R., Nat. Biotechnol., 2006, 24:1241-52; Jefferis, R., Nat. Rev. Drug Discov., 2009, 8 (3): 226-34; Dalziel, M., et al., Science, 2014, 343 (6166): 1235681). Notably, all IgG antibodies are glycosylated in the Fc region thereof on a conserved Asn-297 residue (Alter G., et al., supra).

An Asn-297-linked N-oligosaccharide is comprised of a conserved biantennary core structure (Liu, L., J Pharm Sci., 2015, 104 (6): 1866-84) consisting of two covalently-linked N-acetylglucosamine (GlcNAc) residues, further linked to a mannose, which links in a 1,3- and 1,6-branching manner to two other mannose residues (Alter, G., et al., supra). Additional monosaccharides, including two galactoses, a fucose, a bisecting GlcNAc, and two sialic acids (Alter, G., et al., supra), may extend the core structure, giving rise to considerable structural and functional heretogeneity (Jefferis, R., Biochem J., 1990, 268 (3): 529-37; Rudd, P. M., Science, 2001, 291 (5512): 2370-6; Liu, L., supra). At least 30 structures (glycoforms) for IgG Asn-297-linked N-oligosaccharides have been reported (Alter, G., et al., supra).

Antibodies expressed in mammalian cells are usually more than 80% fucosylated (Kamoda, S., et al., J Chromatogr A., 2004, 1050 (2): 211-6; Shinkawa, T., et al., J Biol Chem., 2003, 278 (5): 3466-73). For example, normal Chinese Hamster Ovary (CHO) cells and HEK293 cells add fucose to 80-98% of Asn-297-linked N-oligosaccharides to IgG antibodies (Shields, R. L. et al., J Biol Chem., 2002, 277 (30): 26733-40).

In one aspect, provided herein is a GPRC5D antibody having no fucose in the oligossacharide attached to its Fc region and having RE mutations in the Fc region. In another aspect, provided herein is a GPRC5D antibody having no fucose in the oligossacharide attached to its Fc region and having RE mutations in the Fc region.

Standard techniques known to those of skill in the art, e.g., mass spectrometry, can be used to characterize the Asn297-linked N-oligosaccharides on the antibodies (Pereira, N. A., et al., supra; Shields, R. L. et al., supra). For example, in a matrix-assisted laser desorption/ionization time-of-flight mass spectral (MALDI-TOF-MS) analysis, 50 mg of IgG antibodies were immobilized in MultiScreen 96-well IP plates (Millipore) to polyvinylidene difluoride membranes. Proteins were then reduced using 50 mL of a 0.1 M solution of DTT in RCM buffer (pH 8.6, 3.2 mM EDTA, 360 mM Tris, and 8 M urea). Next, they were incubated in the dark for 30 minutes at 25° C. in RCM buffer containing 0.1 M iodoacetic acid, in order to carboxymethylate the free sulfhydryl groups resulting from the reduction step. Membrane-bound proteins were then incubated for 1 hour at 25° C. in a 1% solution of polyvinylpyrrolidone 360 (Sigma) in water, and their oligosaccharides were cleaved from the proteins by a three-step process: incubation for 3 hours at 37° C. in pH 8.4 Tris acetate buffer (25 mL) containing 32 units of peptide: N-glycosidase F (New England Biolabs, Beverly, MA), addition of 1.5 M acetic acid (2.5 mL) to lower the pH, and incubation for 3 hours at 25° C. (Shields, R. L. et al., J Biol Chem., 2001, 276 (9): 6591-604).

In some embodiments, the GPRC5D antibodies provided herein are produced by expressing a polynucleotide encoding the GPRC5D antibodies or a fragment thereof in a host cell that is deficient in adding a fucose to an oligosaccharide attached to an antibody.

In mammalian cells, FUT8 encodes the only enzyme, α-1,6 fucosyltransferase, that catalyzes core fucosylation, the transfer of a GDP-fucose residue to the innermost GlcNAc via α-1,6-linkage (Imai-Nishiya, H., et al., BMC Biotechnol., 2007, 7:84). Oligosaccharide fucosylation requires intracellular GDP-fucose as substrate, which is synthesized via the de novo pathway or the salvage pathway in the cytoplasm. In the de novo pathway, GDP-mannose 4,6-dehydratase (GMD) mediates the synthesis of GDP-4-keto-6-deoxy-mannose (GKDM) from GDP-mannose, followed by the synthesis of GDP-fucose mediated by GDP-keto-6-deoxymannose 3,5-epimerase, 4-reductase (FX) (Imai-Nishiya, H., et al., supra). As such, cell lines with deficient GMD enzymes, e.g., CHO Lec 13 cells, or reduced α-1,6 fucosyltransferase activity resulting from mutated FUT8 genes, have been shown to generate afucosylated antibodies (Pereira, N. A., et al., Mabs, 2018, 10 (5): 693-711). For example, antibodies with approximately 10% fucosylation (Shields, R. L. et al., supra) or less can be consistently produced in Lec13 cells (Shields, R. L. et al., supra; Kanda Y., Biotechnol Bioeng., 2006, 94 (4): 680-8), while increased fucosylation may occur when cells are cultured in a static flask to confluence (Percira, N. A., et al., supra).

The addition of a bisecting GlcNAc to the oligosaccharide core structure creates steric hindrance for fucosylation (Alter, G., et al., supra). As such, overexpression of β-1,4-mannosyl-glycoprotein 4-β-N-acetylglucosaminyltransferase (GnT-III), which catalyzes the addition of a bisecting GlcNAc to the innermost mannose, was shown to dramatically reduce Fc fucosylation (Pereira, N. A., et al., supra).

Moreover, inactivated Golgi GDP-fucose transporter (GFT) gene (Slc35cl) has been shown to produce afucosylated antibodies, e.g., in CHO-gmt3 cells (Pereira, N. A., et al., supra). Use of biochemical inhibitors of fucosylation, e.g., fucose analogs such as 2-fluorofucose and 5-alkynylfucose, can also generate afucosylated antibodies (Percira, N. A., et al., supra). The intermediate GKDM in the de novo fucose synthesis pathway in mammalian cells can be reduced by bacteria GDP-4-keto-6-deoxy mannose reductase (RMD) to GDP-rhamnose, thus bypassing the fucose biosynthesis pathway. Afucosylated antibodies can also be generated in cells in which bacterial RMD is heterologously expressed in the cytosol (Pereira, N. A., et al., supra).

In some embodiments, the antibodies provided herein are produced by expressing the antibodies in a host cell having a deficiency in any of the above mentioned enzymes. In some embodiments, the host cell has reduced GDP-mannose 4,6-dehydratase (GMD) activity. In some embodiments, the host cell has reduced α-1,6 fucosyltransferase activity.

### Polynucleotides

In certain embodiments, the disclosure provides polynucleotides that encode the present antibodies that bind to GPRC5D described herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the antibodies that bind GPRC5D of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the antibody that binds GPRC5D of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGT10, AGT11, AEMBL4, and ANM1149, AZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, VA, CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-KISV (Lonza Biologics, Walkersville, MD), CHO-KI (ATCC CRL-61) or DG44.

### Preparation of Antibodies and Method of Making

Methods of preparing antibodies have been described. Sec, e.g., Els Pardon et al, Nature Protocol, 9 (3): 674 (2014). Antibodies (such as scFv fragments) may be obtained using methods known in the art such as by immunizing a Camelid species (such as camel or llama) and obtaining hybridomas therefrom, or by cloning a library of antibodies using molecular biology techniques known in the art and subsequent selection by ELISA with individual clones of unselected libraries or by using phage display.

GPRC5D Antibodies provided herein may be produced by culturing cells transformed or transfected with a vector containing an antibody-encoding nucleic acids. Polynucleotide sequences encoding polypeptide components of the GPRC5D antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. GPRC5D antibodies produced by the host cells are purified using standard protein purification methods as known in the art.

Methods for antibody production including vector construction, expression, and purification are further described in Pluckthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubcai ed., 2011); and Therapeutic Monoclonal Antibodies: From Bench to Clinic (An ed., 2009).

It is, of course, contemplated that alternative methods, which are well known in the art, may be employed to prepare anti-GPRC5D antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the anti-GPRC5D antibody may be chemically synthesized separately and combined using chemical or enzymatic methods to produce the desired anti-GPRC5D antibody. Alternatively, antibodies or fusion construct may be purified from cells or bodily fluids, such as milk, of a transgenic animal engineered to express the antibody, as disclosed, for example, in U.S. Pat. Nos. 5,545,807 and 5,827,690.

### Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or RIN═C═NR, where R and RI are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, an appropriate host animal is immunized to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Goding, Monoclonal Antibodies: Principles and Practice, Academic Press (1986), pp. 59-103. Immortalized cell lines are usually transformed mammalian cells. The hybridoma cells thus prepared are seeded and grown in a suitable culture medium that preferably contains one or more substances that inhibit the growth or survival of the unfused, parental myeloma cells. Preferred immortalized myeloma cells are those that fuse efficiently, support stable high-level production of antibody by the selected antibody-producing cells, and are sensitive to a medium such as HAT medium.

Culture medium in which hybridoma cells are growing is assayed for production of monoclonal antibodies directed against the antigen. The culture medium in which the hybridoma cells are cultured can be assayed for the presence of monoclonal antibodies directed against the desired antigen. Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991). Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence (U.S. Pat. No. 4,816,567; Morrison, et al., Proc. Natl Acad. Sci. USA, 81:6851 (1984)), or by covalently joining to the coding sequence all or part of the coding sequence for a non-immunoglobulin polypeptide. Such non-immunoglobulin polypeptides can be substituted to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Recombinant Production in Prokaryotic Cells

Polynucleic acid sequences encoding the antibodies of the present disclosure can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present antibody by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, thegalactamase and lactose promoter systems, a tryptophan (trp) promoter system and hybrid promoters such as the tac or the tre promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20:269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the GPRC5D antibodies according to the present disclosure can occur in the cytoplasm of the host cell, and therefore does not require the presence of secretion signal sequences within each cistron. Certain host strains (e.g., the E. coli trxB strains) provide cytoplasm conditions that are favorable for disulfide bond formation, thereby permitting proper folding and assembly of expressed protein subunits.

Prokaryotic host cells suitable for expressing the antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A (nmpc-fepE) degP41 kanR (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the GPRC5D antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods 263:133-147 (2002)). A variety of other inducers may be used, according to the vector construct employed, as is known in the art.

The expressed GPRC5D antibodies of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. To improve the production yield and quality of the antibodies of the present disclosure, various fermentation conditions can be modified. For example, the chaperone proteins have been demonstrated to facilitate the proper folding and solubility of heterologous proteins produced in bacterial host cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Aric et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. Nos. 5,264,365; 5,508,192; Hara et al., Microbial Drug Resistance, 2:63-72 (1996). E. coli strains deficient for proteolytic enzymes and transformed with plasmids overexpressing one or more chaperone proteins may be used as host cells in the expression system encoding the antibodies of the present application.

The GPRC5D antibodies produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. The solid phase to which Protein A is immobilized is preferably a column comprising a glass or silica surface, more preferably a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally, the antibodies of interest are recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the antibodies of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for antibodies production and cultured in conventional nutrient media modified as appropriate for inducing promoters, selecting transformants, or amplifying the genes encoding the desired sequences.

The host cells used to produce the antibodies of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly (styrene-divinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography.

### Pharmaceutical Compositions

In one aspect, the present disclosure further provides pharmaceutical compositions comprising at least one GPRC5D antibody, or antigen binding fragment thereof, of the present disclosure. In some embodiments, a pharmaceutical composition comprises therapeutically effective amount of a GPRC5D antibody or antigen binding fragment thereof provided herein and a pharmaceutically acceptable excipient.

Pharmaceutical compositions comprising a GPRC5D antibody or antigen binding fragment thereof are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The GPRC5D antibody, or antigen binding fragment thereof, of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

A GPRC5D antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof or fusion construct as described herein.

### Methods of Using the Gprcsd Antibodies

In one aspect, provided herein is a method of inhibiting the growth or proliferation of a multiple myeloma comprising administering to the subject the GPRC5D antibodies provided herein to inhibit the growth or proliferation of the multiple myeloma.

In some embodiments, the GPRC5D antibody provided herein inhibiting the growth or proliferation of a multiple myeloma by at least about 10%. In some embodiments, the GPRC5D antibody provided herein inhibiting the growth or proliferation of a multiple myeloma by at least about 20%. In some embodiments, the GPRC5D antibody provided herein inhibiting the growth or proliferation of a multiple myeloma by at least about 30%. In some embodiments, the GPRC5D antibody provided herein inhibiting the growth or proliferation of a multiple myeloma by at least about 40 In some embodiments, the GPRC5D antibody provided herein inhibiting the growth or proliferation of a multiple myeloma by at least about 50%. In some embodiments, the GPRC5D antibody provided herein inhibiting the growth or proliferation of a multiple myeloma by at least about 60%. In some embodiments, the GPRC5D antibody provided herein inhibiting the growth or proliferation of a multiple myeloma by at least about 70%. In some embodiments, the GPRC5D antibody provided herein inhibiting the growth or proliferation of a multiple myeloma by at least about 80%. In some embodiments, the GPRC5D antibody provided herein inhibiting the growth or proliferation of a multiple myeloma by at least about 90%. In some embodiments, the GPRC5D antibody provided herein inhibiting the growth or proliferation of a multiple myeloma by at least about 95%.

In another aspect, provided herein is a method of treating a multiple myeloma comprising administering to the subject a GPRC5D antibody or antigen binding fragment thereof provided herein.

Methods of administration and dosing is described in more detail below.

In another aspect, provided herein is the use of the GPRC5D antibody or antigen binding fragment provided herein in the manufacture of a medicament for treating a disease or disorder in a subject.

In another aspect, provided herein is the use of a pharmaceutical composition provided herein in the manufacture of a medicament for treating a multiple myeloma in a subject.

In another aspect, provided herein is the use of a GPRC5D antibody or antigen binding fragment thereof provided herein in the manufacture of a medicament, wherein the medicament is for use in a method for detecting the presence of a GPRC5D in a biological sample, the method comprising contacting the biological sample with the GPRC5D antibody under conditions permissive for binding of the GPRC5D antibody to the GPRC5D protein, and detecting whether a complex is formed between the antibody and the GPRC5D protein.

In other aspects, the antibodies and fragments thereof or fusion construct of the present disclosure are useful for detecting the presence of GPRC5D in a biological sample. The term “detecting” as used herein encompasses quantitative or qualitative detection. In certain embodiments, a biological sample comprises bodily fluid, a cell, or a tissue. Diagnostic assays and methods are described in more detail below.

### Methods of Administration and Dosing

In a specific embodiment, provided herein is a composition for use in the prevention and/or treatment of a multiple myeloma comprising a GPRC5D antibody or antigen binding fragment thereof provided herein. In one embodiment, provided herein is a composition for use in the treatment of a multiple myeloma, wherein the composition comprises a GPRC5D antibody or antigen binding fragment thereof provided herein. In certain embodiments, the subject is a subject in need thereof. In some embodiments, the subject has the multiple myeloma.

Also provided herein are methods of treating a multiple myeloma by administrating to a subject of a GPRC5D antibody or antigen binding fragment thereof provided herein, or pharmaceutical composition comprising an antibody or antigen binding fragment thereof provided herein. In one aspect, the GPRC5D antibody or antigen binding fragment thereof is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In a one embodiment, the subject is a human. In another embodiment, the subject is a human with a multiple myeloma.

Various delivery systems are known and can be used to administer a prophylactic or therapeutic agent (e.g., an antibody or antigen binding fragment thereof provided herein). The prophylactic or therapeutic agents, or compositions may be administered by any convenient route. Administration can be systemic or local.

In a specific embodiment, it may be desirable to administer a prophylactic or therapeutic agent, or a pharmaceutical composition provided herein locally to the area in need of treatment.

In a specific embodiment, where the composition provided herein is a nucleic acid encoding a prophylactic or therapeutic agent (e.g., an antibody or antigen binding fragment thereof provided herein), the nucleic acid can be administered in vivo to promote expression of its encoded prophylactic or therapeutic agent, by constructing it as part of an appropriate nucleic acid expression vector and administering it so that it becomes intracellular. Alternatively, a nucleic acid can be introduced intracellularly and incorporated within host cell DNA for expression by homologous recombination.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle.

In certain embodiments, the GPRC5D antibody or antigen binding fragment thereof provided herein are administered prophylactically or therapeutically to a subject. The GPRC5D antibody or antigen binding fragment thereof provided herein can be prophylactically or therapeutically administered to a subject so as to prevent, lessen or ameliorate a multiple myeloma or symptom thereof.

Any embodiments or aspects of the disclosure, which in the description or in the claims refer to a method of treatment, are applicable to the manufacture of a medicament for the treatment mutatis mutandis.

Any embodiments or aspects of the disclosure, which in the description or in the claims refer to a method of treatment, are applicable to the compound, composition or pharmaceutical composition for use in the treatment mutatis mutandis.

### Diagnostic Assays and Methods

Labeled antibodies, derivatives, and analogs thereof, which specifically bind to a GPRC5D antigen can be used for diagnostic purposes to detect, diagnose, or monitor a GPRC5D mediated disease. Thus, provided herein are methods for the detection of a GPRC5D-mediated disease comprising: (a) assaying the expression of a GPRC5D antigen in cells or a tissue sample of a subject using one or more antibodies, provided herein that specifically bind to the GPRC5D antigen; and (b) comparing the level of the GPRC5D antigen with a control level, e.g., levels in normal tissue samples (e.g., from a patient not having a GPRC5D-mediated disease, or from the same patient before disease onset), whereby an increase in the assayed level of GPRC5D antigen compared to the control level of the GPRC5D antigen is indicative of a GPRC5D-mediated disease.

Also provided herein is a diagnostic assay for diagnosing a GPRC5D-mediated disease comprising: (a) assaying for the level of a GPRC5D antigen in cells or a tissue sample of an individual using one or more antibodies or fusion constructs, provided herein that immunospecifically bind to a GPRC5D antigen; and (b) comparing the level of the GPRC5D antigen with a control level, e.g., levels in normal tissue samples, whereby an increase in the assayed GPRC5D antigen level compared to the control level of the GPRC5D antigen is indicative of a GPRC5D-mediated disease. In certain embodiments, provided herein is a method of treating a GPRC5D-mediated disease in a subject, comprising: (a) assaying for the level of a GPRC5D antigen in cells or a tissue sample of the subject using one or more antibodies, provided herein that specifically bind to a GPRC5D antigen; and (b) comparing the level of the GPRC5D antigen with a control level, e.g., levels in normal tissue samples, whereby an increase in the assayed GPRC5D antigen level compared to the control level of the GPRC5D antigen is indicative of a GPRC5D-mediated disease. In some embodiments, the method further comprises (c) administering an effective amount of an antibody or fusion constructs, provided herein to the subject identified as having the GPRC5D-mediated disease. A more definitive diagnosis of a GPRC5D-mediated disease may allow health professionals to employ preventative measures or aggressive treatment earlier thereby preventing the development or further progression of the GPRC5D-mediated disease.

Antibodies provided herein can be used to assay GPRC5D antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (121 In), and technetium (99Tc); luminescent labels, such as luminol; and fluorescent labels, such as fluorescein and rhodamine, and biotin.

One aspect provided herein is the detection and diagnosis of a GPRC5D-mediated disease in a human. In one embodiment, diagnosis comprises: a) administering (for example, parenterally, subcutaneously, or intraperitoneally) to a subject an effective amount of a labeled antibody, that specifically binds to a GPRC5D antigen; b) waiting for a time interval following the administering for permitting the labeled antibody to concentrate at sites in the subject where the GPRC5D antigen is expressed (and for unbound labeled molecule to be cleared to background level); c) determining background level; and d) detecting the labeled antibody or fusion constructs, in the subject, such that detection of labeled antibody above the background level indicates that the subject has a GPRC5D-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody or fusion constructs, will then accumulate at the location of cells which contain the specific protein. In vivo tumor imaging is described in S.W. Burchiel et al., “Immunopharmacokinetics of Radiolabeled Antibodies and Their Fragments.” (Chapter 13 in Tumor Imaging: The Radiochemical Detection of Cancer, S.W. Burchiel and B.A. Rhodes, eds., Masson Publishing Inc. (1982).

Depending on several variables, including the type of label used and the mode of administration, the time interval following the administration for permitting the labeled antibody or fusion constructs, to concentrate at sites in the subject and for unbound labeled antibody or fusion constructs, to be cleared to background level is 6 to 48 hours or 6 to 24 hours or 6 to 12 hours. In another embodiment the time interval following administration is 5 to 20 days or 5 to 10 days.

In one embodiment, monitoring of a GPRC5D-mediated disease is carried out by repeating the method for diagnosing the a GPRC5D-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

Kits

Also provided herein are kits comprising an antibody (e.g., an anti-GPRC5D antibody) provided herein, or a composition (e.g., a pharmaceutical composition), or a fusion construct thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that specifically bind to a GPRC5D antigen. In specific embodiments, provided herein are panels of antibodies having different association rate constants different dissociation rate constants, different affinities for GPRC5D antigen, and/or different specificities for a GPRC5D antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30,5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

### Enumerated Embodiments

A number of embodiments of the invention have been described. Nevertheless, it will be understood that various modifications may be made without departing from the spirit and scope of the invention. Accordingly, the following examples are intended to illustrate but not limit the scope of invention described in the claims

Embodiment 1 is an antibody or an antigen-binding fragment specifically binding to GPRC5D, comprising:

a. a heavy chain complementarity determining region 1 (CDR1), a heavy chain complementarity determining region 2 (CDR2), and a heavy chain complementarity determining region 3 (CDR3) comprising the amino acid sequences of SEQ ID NO: 6, 7, and 8, respectively; and a light chain complementarity determining region 1 (CDR1), a light chain complementarity determining region 2 (CDR2), and a light chain complementarity determining region 3 (CDR3) comprising the amino acid sequences of SEQ ID NO: 9, 10, and 11, respectively;

b. a heavy chain CDR1, a heavy chain CDR2, and a heavy chain CDR3 comprising the amino acid sequences of SEQ ID NO: 12, 13, and 8, respectively; and

a light chain CDR1, a light chain CDR2, and a light chain CDR3 comprising the amino acid sequences of SEQ ID NO: 9, 10, and 11, respectively;

c. a heavy chain CDR1, a heavy chain CDR2, and a heavy chain CDR3 comprising the amino acid sequences of SEQ ID NO: 14, 15, and 8, respectively; and

a light chain CDR1, a light chain CDR2, and a light chain CDR3 comprising the amino acid sequences of SEQ ID NO: 9, 10, and 11, respectively;

d. a heavy chain CDR1, a heavy chain CDR2, and a heavy chain CDR3 comprising the amino acid sequences of SEQ ID NO: 16, 17, and 18, respectively; and

a light chain CDR1, a light chain CDR2, and a light chain CDR3 comprising the amino acid sequences of SEQ ID NO: 19, AAS, and 11, respectively; or e. a heavy chain CDR1, a heavy chain CDR2, and a heavy chain CDR3 comprising the amino acid sequences of SEQ ID NO: 21, 22, and 23, respectively; and a light chain CDR1, a light chain CDR2, and a light chain CDR3 comprising the amino acid sequences of SEQ ID NO: 24, 25, and 26, respectively.

Embodiment 2 is the antibody or the antigen-binding fragment of embodiment 1, comprising a heavy chain variable region (VH) comprising an amino acid sequence that is at least 90%, 91%, 92%, 93%, 94%, 95%, 96%, 97%, 98%, or 99% identical to SEQ ID NO: 1.

Embodiment 3 is the antibody or the antigen-binding fragment of embodiment 1 or 2, comprising a VH comprising SEQ ID NO: 1.

Embodiment 4 is the antibody or the antigen-binding fragment of any one of embodiments 1 to 3, comprising a light chain variable region (VL) comprising an amino acid sequence that is at least 90%, 91%, 92%, 93%, 94%, 95%, 96%, 97%, 98%, or 99% identical to SEQ ID NO: 2.

Embodiment 5 is the antibody or the antigen-binding fragment of any one of embodiments 1 to 4, comprising a VL comprising SEQ ID NO: 2.

Embodiment 6 is the antibody or the antigen-binding fragment of any one of embodiments 1 to 5, comprising a heavy chain variable region (VH) comprising SEQ ID NO: 1, and a light chain variable region (VL) comprising SEQ ID NO: 2.

Embodiment 7 is the antibody or the antigen-binding fragment of any one of embodiments 1 to 6, wherein the antibody or antigen-binding fragment is an IgG.

Embodiment 8 is the antibody or the antigen-binding fragment of any one of embodiments 1 to 7, wherein the antibody, or antigen-binding fragment comprises an IgG1 isotype Fc region.

Embodiment 9 is the antibody or the antigen-binding fragment of any one of embodiments 1 to 8, wherein the IgG1 isotype Fc region comprises K248E and T437R (RE) mutations as per the EU numbering system.

Embodiment 10 is the antibody or the antigen-binding fragment of any one of embodiments 1 to 9, wherein the antibody or the antigen-binding fragment is afucosylated.

Embodiment 11 is the antibody or the antigen-binding fragment of embodiment 10, wherein the antibody or the antigen-binding fragment has enhanced antibody-dependent cellular cytotoxicity (ADCC) activity and enhanced complement-dependent cytotoxicity (CDC) activity.

Embodiment 12 is the antibody or the antigen-binding fragment of claim 10, wherein the antibody or the antigen-binding fragment has enhanced antibody-dependent cellular cytotoxicity (ADCC) activity as compared with a fucosylated antibody or an antigen-binding fragment thereof.

Embodiment 13 is the antibody or the antigen-binding fragment of any one of embodiments 1 to 12, wherein the antibody or the antigen-binding fragment has antibody-dependent cellular phagocytosis (ADCP) activity.

Embodiment 14 is the antibody or the antigen-binding fragment of any one of embodiments 1 to 13, wherein the antibody or the antigen-binding fragment further comprises knob-into-hole (KiH) mutations.

Embodiment 15 is the antibody or the antigen-binding fragment of any one of embodiments 1 to 14, wherein the IgG1 isotype Fc region further comprises H435R and Y436F mutations per the EU numbering system.

Embodiment 16 is a monovalent antibody, or an antigen-binding fragment thereof, of any one of embodiments 1 to 15.

Embodiment 17 is the monovalent antibody or the antigen-binding fragment of embodiment 16, wherein the monovalent antibody or the antigen-binding fragment comprises an Fc domain and a Fab.

Embodiment 18 is the monovalent antibody or the antigen-binding fragment of embodiment 16 or embodiment 17, wherein the monovalent antibody or the antigen-binding fragment comprises a first heavy chain (HCl), a second heavy chain (HC2), and a second light chain (LC2).

Embodiment 19 is the monovalent antibody or the antigen-binding fragment of any one of embodiments 16 to 18, wherein the HCl comprises an amino acid sequence that is at least 90%, 91%, 92%, 93%, 94%, 95%, 96%, 97%, 98%, or 99% identical to SEQ ID NO: 3.

Embodiment 20 is the monovalent antibody or the antigen-binding fragment of any one of embodiments 16 to 19, wherein the HCl comprises SEQ ID NO: 3.

Embodiment 21 is the monovalent antibody or the antigen-binding fragment of any one of embodiments 16 to 20, wherein the HC2 comprises an amino acid sequence that is at least 90%, 91%, 92%, 93%, 94%, 95%, 96%, 97%, 98%, or 99% identical to SEQ ID NO: 4.

Embodiment 22 is the monovalent antibody or the antigen-binding fragment of any one of embodiments 16 to 21, wherein the HC2 comprises SEQ ID NO: 4.

Embodiment 23 is the monovalent antibody or the antigen-binding fragment of any one of embodiments 16 to 22, wherein the LC2 comprises an amino acid sequence that is at least 90%, 91%, 92%, 93%, 94%, 95%, 96%, 97%, 98%, or 99% identical to SEQ ID NO: 5.

Embodiment 24 is the monovalent antibody or the antigen-binding fragment of any one of embodiments 16 to 23, wherein the LC2 comprises SEQ ID NO: 5.

Embodiment 25 is a monovalent antibody or an antigen-binding fragment specifically binding to GPRC5D, comprising a first heavy chain comprising SEQ ID NO: 3, a second heavy chain comprising SEQ ID NO: 4 and a second light chain comprising SEQ ID NO: 5.

Embodiment 26 is a synthetic polynucleotide encoding the antibody, the monovalent antibody, or the antigen-binding fragment of any one of embodiments 1-24.

Embodiment 27 is a vector comprising the synthetic polynucleotide of embodiment 26.

Embodiment 28 is a host cell comprising the synthetic polynucleotide of embodiment 26 or the vector of embodiment 27.

Embodiment 29 is the host cell of embodiment 28, wherein the host cell lacks a fucosylation capability.

Embodiment 30 is a pharmaceutical composition comprising the antibody, the monovalent antibody, or the antigen-binding fragment of any one of embodiments 1 to 25 and a pharmaceutically acceptable carrier.

Embodiment 31 is a method for generating the antibody, the monovalent antibody, or the antigen-binding fragment of any one of embodiments 1 to 25 by culturing the host cell of embodiment 27.

Embodiment 32 is an antibody, a monovalent antibody, or an antigen-binding fragment produced by the method of embodiment 31.

Embodiment 33 is the antibody, the monovalent antibody, or the antigen-binding fragment of any one of embodiments 1 to 25, wherein the antibody or the antigen-binding fragment is produced by expressing the synthetic polynucleotide encoding the antibody, the monovalent antibody, or the antigen-binding fragment in a host cell that lacks afucosylation capability.

Embodiment 34 is a method of inhibiting the growth or proliferation of a multiple myeloma, the method comprising administering to a subject the antibody, the monovalent antibody, or the antigen-binding fragment of any one of embodiments 1 to 25 to inhibit the growth or proliferation of the multiple myeloma.

Embodiment 35 is a method of treating a multiple myeloma, the method comprising administering to a subject in need thereof the antibody, the monovalent antibody or the antigen-binding fragment of any one of embodiments 1 to 25, to the subject for a time sufficient to treat the multiple myeloma.

Embodiment 36 is the method of embodiment 34 or embodiment 35, wherein the multiple myeloma is characterized by the presence of GPRC5D.

Embodiment 37 is a kit comprising the antibody, the monovalent antibody, or the antigen-binding fragment of any one of embodiments 1 to 25 and packaging for the same.

Embodiment 38 is a kit comprising the synthetic polynucleotide of embodiment 26 and packaging for the same.

Embodiment 39 is a kit comprising the antibody, the monovalent antibody, or antigen-binding fragment of any one of embodiments 1 to 25, the polynucleotide of embodiment 26, and packaging for the same.

Any references in the description or in the claims to methods of treatment refer to the compounds, compositions, pharmaceutical compositions and medicaments for use in a method of treatment of the human (or animal) body by therapy (or for diagnosis).

Any references in the description or in the claims to methods of treatment refer to the use of the compounds, compositions, pharmaceutical compositions for the manufacture of a medicament for the treatment of the human (or animal) body by therapy (or for diagnosis).

### Examples

### Example 1. Comprehensive Immunization Strategies and Innovative Approaches for Generating Anti-GPRC5D Antibodies in Rodent Models

OMT Omnirats (female, rat 3, 4, 5, 9, and 10) underwent weekly immunizations with human GPRC5D (huGPRC5D) DNA over a span of 6 weeks. The immunizations involved intramuscular injections with electroporation in each tibialis muscle, with each injection containing 200 μg plasmids at 5 mg/ml coding GPRC5D (G-protein coupled receptor family C group 5 member D [Homo sapiens], Sequence ID: NP_061124.1). Rats 9 and 10 additionally received 6 weekly boosts of CpG Adjuvant (InvivoGen ODN1826, Lot V3902-01T; 2 mg/mL in 500μ L). As a final cell-boost, each rat received RBL-2H3 rat fibroblast cells (ATCC CRL-2256) expressing huGPRC5D (2 million cells IV and 4 million cells IP).

Immune sera samples were collected twice during the immunization process (Days 32 and 46) and subjected to binding tests via flow cytometry. The tests were performed using HEK293F cells (Thermofisher R79007) expressing huGPRC5D and HEK293F parental cells (negative control).

On day 46, lymph nodes were harvested from each rat, pooled, homogenized into a single-cell suspension, and lymphocytes were fused with FO (Follicular B cells) mouse myeloma cell line (ATCC CRL-1646). Hybridoma cells were cultured for 7-14 days.

Hybridoma supernatants were screened via cell MSD on HEK293F cells expressing huGPRC5D to detect secreted anti-GPRC5D antibodies. Primary hits were identified as samples with assay signals greater than 5.85-fold the negative control average. A total of 176 samples meeting this criterion were scaled up and re-screened by flow cytometry for binding to MM.1R (ATCC CRL-2975) cells expressing GPRC5D and HEK293F cells expressing huGPRC5D but not HEK293F parental cells. Of these, 108 hits with the desired binding profile were security frozen and advanced for variable region cloning. The variable regions were subsequently sequenced and expressed as human IgG1 antibodies for further characterization.

In addition to the described immunization campaign, other strategies were explored to generate anti-GPRC5D antibodies. Ablexis mice, Sprague-Dawley rats, and OMT1 rats were all immunized following a protocol similar to HYB:621 to assess immune responses across rodent strains. Blood was drawn for sera titrations on day 33, and on day 43, lymph nodes from select rodents were harvested for hybridoma fusion. Hybridoma colonies were cultured, supernatants were harvested, and screening was performed by cell MSD on HEK293F cells expressing huGPRC5D. Confirmed hits were further validated for binding by flow cytometry to MM.1R cells and HEK293F cells expressing huGPRC5D. Another round of immunizations was conducted using Ablexis mice, comparing GPRC5D DNA constructs, route of injection, and electroporation strategy.

### Example 2. A Novel Format Conversion Implemented to Generate Antibodies Exhibiting Optimal Complement-Dependent Cytotoxicity (CDC) Activities

Effective complement-dependent cytotoxicity (CDC) of a therapeutic antibody relies on the optimal recruitment of complement component 1q (Clq) and the formation of a hexameric Fc arrangement. The potency of CDC activity, particularly in the context of anti-CD20 therapeutic antibodies, is influenced by the interplay of stoichiometry, binding angle, and the flexibility of the Fab arm. These determinants are challenging to predict, making traditional screening of binding and CDC function on bivalent monoclonal antibodies (mAbs) insufficient for generating hits with optimal CDC activities.

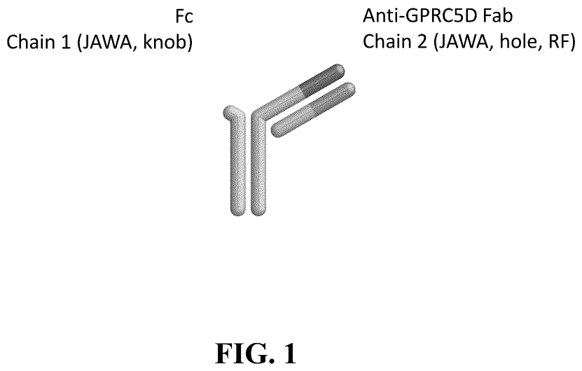

To address this limitation and effectively screen molecules with the greatest CDC potential, a novel molecular formatting approach was employed for binders selected from various immunization campaigns. The monovalent antibody features an Fc region paired with an anti-GPRC5D heavy and light chain (FIG. 1), enhancing heterodimerization through the use of knobs-into-holes mutations. Heavy chain 1 includes the “knob” mutation T366W (EU numbering), while heavy chain 2 features the anti-GPRC5D Fab region and “hole” mutations T366S, L368A, Y407V, along with additional mutations H435R and Y436F to disrupt protein A binding. Both heavy chains incorporate K248E and T437R, or “RE” (also known as “JAWA”), mutations in the constant region (Fc) designed to enhance antibody clustering on target cells, leading to improved complement-dependent cytotoxicity.

Antibodies were produced using either the CHO-Expi cell line, yielding a typical Fc glycan, or a Fut8 knockout cell line resulting in an Fc with fully afucosylated core glycan. Afucosylation enhances binding to Fcγ receptors, particularly FcgRIIIa (CD16), and to a lesser extent FegRIIa, leading to enhanced effector functions, primarily antibody-dependent cell-mediated cytotoxicity (ADCC) and antibody-dependent cellular phagocytosis (ADCP).

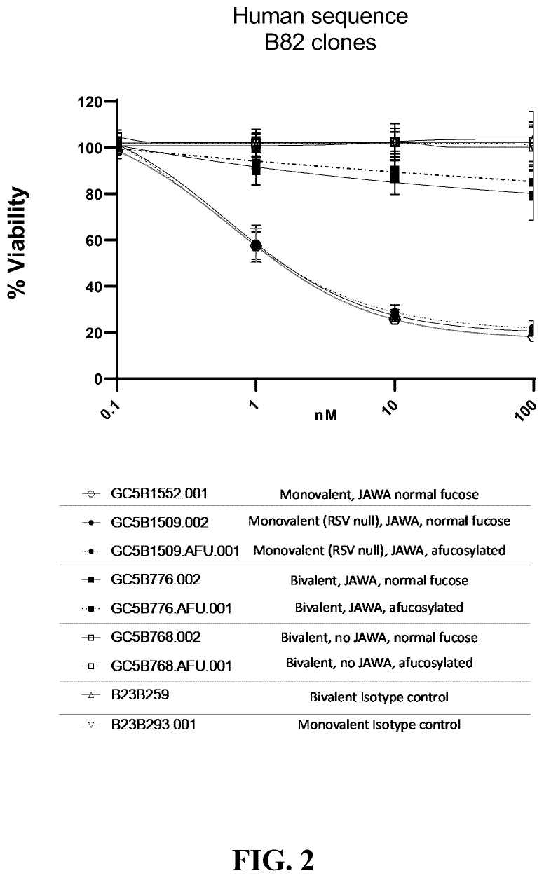

FIG. 2 illustrates that generating a monoclonal antibody against GPRC5D alone is insufficient for inducing CDC mechanisms to lyse tumor cells. While the bivalent antibody showed no CDC activity, the K248E and T437R, or “RE” mutations induced some CDC. However, a remarkable increase in CDC activity is observed when the molecular formatting combines the right binder, JAWA mutation, and monovalency. This molecular design is compatible with afucosylation, and molecules produced in both normal CHO cells and Fut8 knockout cells exhibit the same potent CDC activity.

### Example 3. Screening for Binding of Monovalent Hits

A selection of 31 hits from various immunization campaigns underwent conversion to the monovalent JAWA format as detailed in Example 2. The selection was based on their sequence diversity, in silico liability analysis, target binding, and thermal stability. These monovalent binders, featuring “JAWA” Fc mutations, were subsequently reevaluated for binding to target cells to eliminate hits that may experience a loss of target binding due to reduced avidity as monovalent binders.

In brief, two endogenous GPRC5D-expressing cell lines, H929 and MM1.R, along with their corresponding GPRC5D knock-out lines, were cultured, harvested, and resuspended in PBS using standard cell culture procedures. The four cell lines were either stained with no fluorescent label, 0.02UM CSFE (BD Pharmingen, #C34554), or 0.2 uM CTV (BD Pharmingen, #C34557), or both labels. Near-infrared live/dead stain (Thermo Fisher, #L10119) was added to all cells at a 1:10,000 dilution for 10-15 minutes at room temperature. Subsequently, cells were centrifuged for 5 minutes at 300g, and FBS (Gibco, #16000-036) was added to the tubes. After another centrifugation, cells were resuspended in R10 media, and all four lines were mixed in equal volumes.

Next, 50,000 cells (cell mixture) in a 50 μL volume per well were placed in 96-well v-bottom polypropylene plates (Greiner Bio, #651261), and antibody dilutions were added to the cells. Following a 1-hour incubation at 370C, cells were washed twice, and 50 ul of 2 μg/ml goat-anti-human Fc AF647 (Jackson IR, #109-606-098) was added to the appropriate wells of the plates, incubating for 30 minutes at 4° C. After two additional washes, cells were read on the Intellicyt iQue Screener Plus (Sartorius). Applying appropriate gating and compensation, the signal-to-background ratio (geometric mean of each population in each well/geometric mean of negative control wells) was plotted to generate cell binding dose-response curves for the GPRC5D monovalent hits No binding was observed of these monovalent hits to either of the GPRC5D knock-out cell lines. Binding parameters of wild type H929 and MM1.R are shown in Table 3.

TABLE 3
Cell binding parameters of monovalent hits

| | H929 | | | | | MM1.R | | | | |
|---|---|---|---|---|---|---|---|---|---|---|
| | EC50 | | | Hill | | EC50 | | | Hill | |
| Name | [M] | Top | Bottom | Slope | EC90(M) | [M] | Top | Bottom | Slope | EC90(M) |
| GC5B768 | 1.3E−10 | 23.7 | 3.3 | 1.8 | 4.5E−10 | 1.9E−10 | 55.3 | 4.4 | 1.1 | 1.5E−09 |
| GC5B749 | 2.9E−10 | 26.9 | 1.6 | 1.3 | 1.6E−09 | 3.7E−10 | 58.1 | 0.9 | 1.0 | 3.6E−09 |
| GC5B1605 | 1.2E−09 | 43.4 | 2.8 | 1.1 | 9.4E−09 | 1.3E−09 | 82.6 | 2.3 | 0.8 | 2.0E−08 |
| GC5B1565 | 1.7E−09 | 43.0 | 3.3 | 1.6 | 6.5E−09 | 1.5E−09 | 82.8 | 5.3 | 1.5 | 6.4E−09 |
| GC5B1555 | 2.6E−09 | 10.9 | 1.5 | 1.6 | 1.0E−08 | 4.2E−09 | 16.2 | 1.6 | 1.9 | 1.3E−08 |
| GC5B1564 | 3.5E−09 | 33.8 | 1.8 | 1.4 | 1.8E−08 | 3.5E−09 | 71.8 | 2.4 | 1.2 | 2.2E−08 |
| GC5B1571 | 2.6E−10 | 39.2 | 6.3 | 1.3 | 1.3E−09 | 4.3E−10 | 93.5 | 8.3 | 1.0 | 4.2E−09 |
| GC5B1563 | 9.1E−09 | 14.2 | 1.2 | 1.6 | 3.5E−08 | 1.5E−08 | 28.9 | 1.4 | 1.4 | 6.7E−08 |
| GC5B1552 | 1.3E−09 | 24.7 | 2.2 | 1.7 | 4.7E−09 | 3.2E−09 | 62.4 | 3.1 | 1.3 | 1.8E−08 |
| GC5B1545 | 5.6E−09 | 21.7 | 1.5 | 1.2 | 3.5E−08 | 9.9E−09 | 42.6 | 1.4 | 1.0 | 8.1E−08 |
| GC5B1537 | 8.7E−09 | 5.8 | 1.0 | 1.3 | 4.9E−08 | 1.2E−08 | 9.8 | 1.1 | 1.6 | 4.7E−08 |
| GC5B1562 | 7.3E−10 | 43.6 | 3.1 | 1.2 | 4.4E−09 | 6.4E−10 | 86.3 | 4.3 | 1.1 | 4.8E−09 |
| GC5B1569 | 4.5E−10 | 39.2 | 4.8 | 1.5 | 2.0E−09 | 6.4E−10 | 90.1 | 7.4 | 1.2 | 3.7E−09 |
| GC5B1561 | 8.3E−10 | 35.7 | 3.1 | 1.6 | 3.4E−09 | 8.5E−10 | 79.8 | 4.7 | 1.3 | 4.7E−09 |
| GC5B1543 | 2.0E−08 | 7.2 | 1.1 | 1.1 | 1.5E−07 | 2.6E−08 | 13.9 | 1.3 | 1.5 | 1.2E−07 |
| GC5B1568 | 2.1E−09 | 31.4 | 2.1 | 1.3 | 1.1E−08 | 2.1E−09 | 68.4 | 3.0 | 1.2 | 1.3E−08 |
| GC5B1560 | 9.6E−09 | 7.1 | 1.1 | 1.9 | 3.1E−08 | 1.3E−08 | 20.2 | 1.3 | 1.8 | 4.4E−08 |
| GC5B1549 | 2.0E−09 | 42.9 | 2.3 | 1.1 | 1.4E−08 | 2.1E−09 | 81.4 | 2.7 | 1.0 | 2.0E−08 |
| GC5B1567 | 1.1E−09 | 39.2 | 2.4 | 1.2 | 7.1E−09 | 1.1E−09 | 83.8 | 4.7 | 1.4 | 5.6E−09 |
| GC5B1557 | 2.1E−09 | 18.7 | 1.6 | 1.0 | 2.0E−08 | 9.5E−08 | 79.8 | −2.2 | 0.3 | 3.2E−04 |
| GC5B1548 | 9.3E−10 | 36.7 | 3.4 | 0.8 | 1.6E−08 | 1.6E−09 | 75.6 | 3.0 | 0.6 | 6.7E−08 |
| GC5B1556 | 1.1E−09 | 19.7 | 2.0 | 1.6 | 4.5E−09 | 1.4E−09 | 36.9 | 2.5 | 1.5 | 5.8E−09 |

### Example 4. Screening and Characterization of Monovalent Hits for CDC, ADCC, and ADCP Activities

### CDC Evaluation:

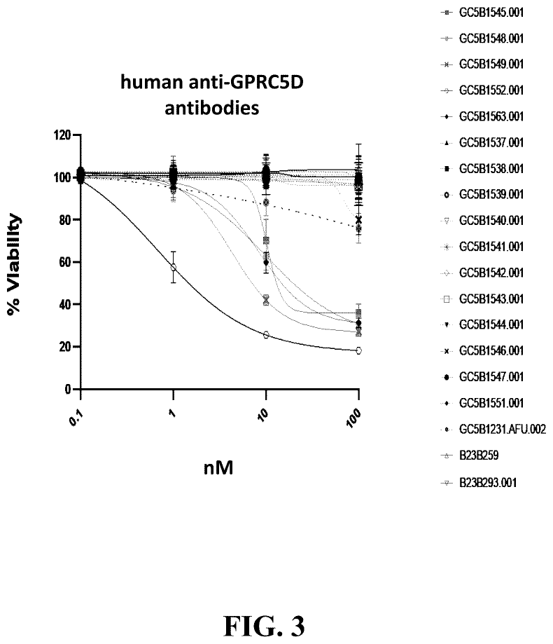

FIG. 3 illustrates the assessment of a panel of 16 monovalent human anti-GPRC5D antibodies for their CDC-inducing activity against the MM.1R cell line using pooled human serum over a 24-hour period. The monovalent isotype control (B23B293.001), bivalent anti-GPRC5D (GC5B1231), and bivalent isotype control (B23B259) antibodies were included as controls, showing no CDC activity. Among the 16 monovalent anti-GPRC5D antibodies tested, only 5 exhibited varying levels of CDC activity.

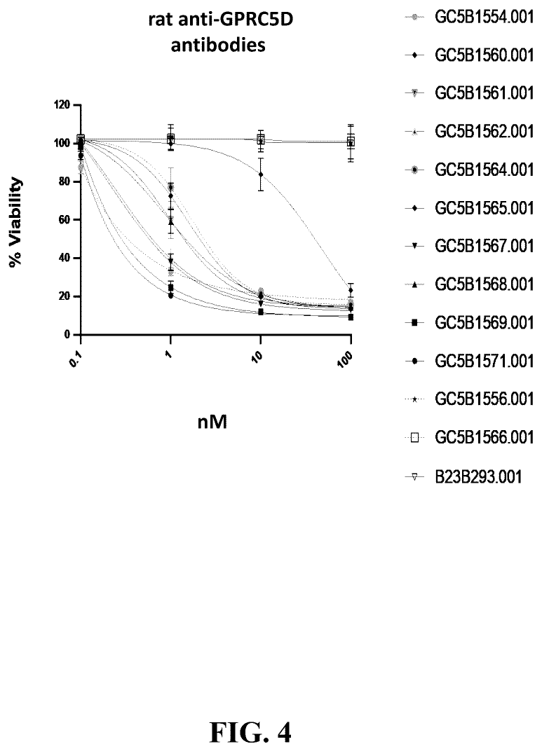

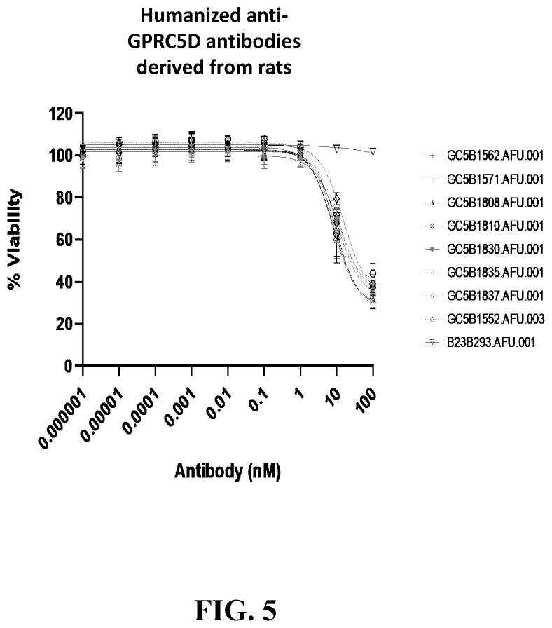

In FIG. 4, a separate panel of 12 monovalent rat anti-GPRC5D antibodies underwent evaluation for CDC-inducing activity against the MM.1R cell line using pooled human serum. The monovalent isotype control (B23B293.001) showed no CDC activity. Out of the 12 monovalent rat anti-GPRC5D antibodies, 10 displayed varying levels of CDC activity. To explore the retention of CDC activity, two rat antibodies (GC5B1562 and GC5B1571) were humanized, resulting in a new panel of monovalent humanized anti-GPRC5D antibodies, as depicted in FIG. 5. All antibodies retained CDC activity, similar to the monovalent anti-GPRC5D antibody GC5B1552.

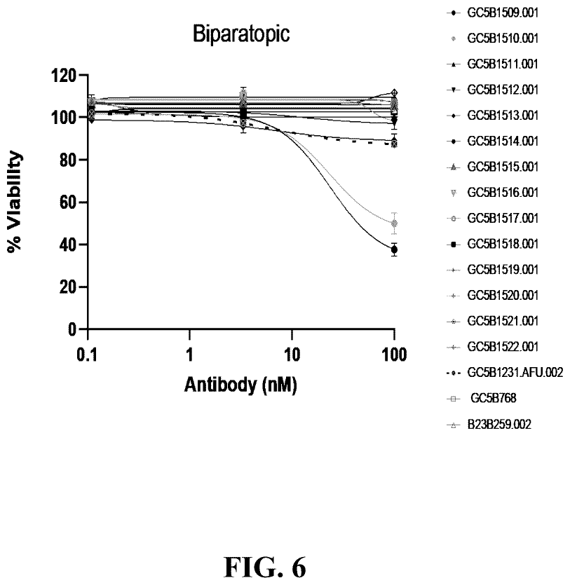

FIG. 6 illustrates the evaluation of bivalent biparatopic anti-GPRC5D antibodies for CDC activity. None of the bivalent and biparatopic antibodies demonstrated substantial CDC activity, except GC5B1509 and GC5B1510. These antibodies were bivalent but contained only one anti-GPRC5D binding region, while the other binder was generated as a null that was not expressed on the surface of MM.1R cells. This confirmed that monovalent binding to GPRC5D was essential for substantial CDC activity against GPRC5D-expressing cells.

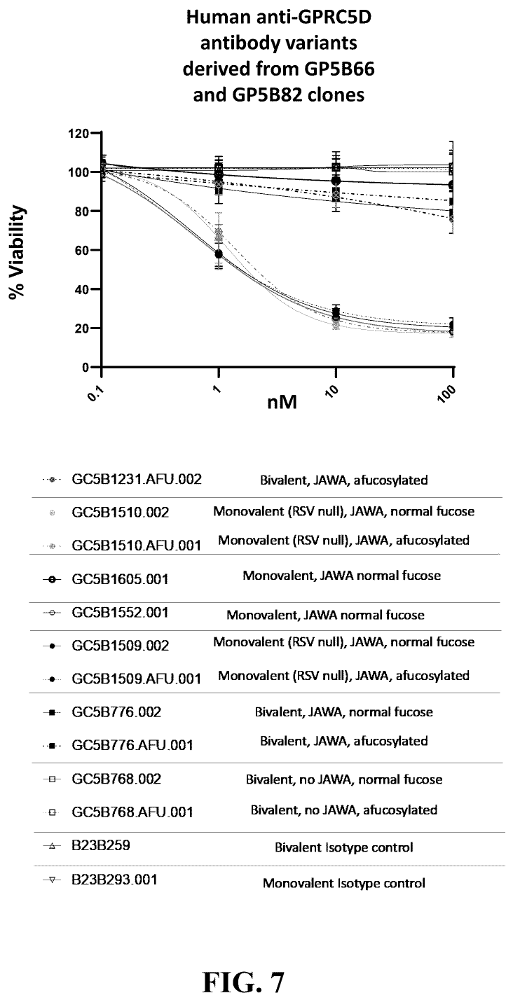

Finally, a comprehensive comparison of all bivalent and monospecific anti-GPRC5D antibodies was conducted, testing them in combinations of CDC-enhancing mutations along with afucosylation against the MM.1R cell line. In all combinations, only the monospecific antibodies (GC5B1509 and GC5B1510) and CDC-enhancing antibodies demonstrated robust CDC activity, as illustrated in FIG. 7.

### ADCC Evaluation:

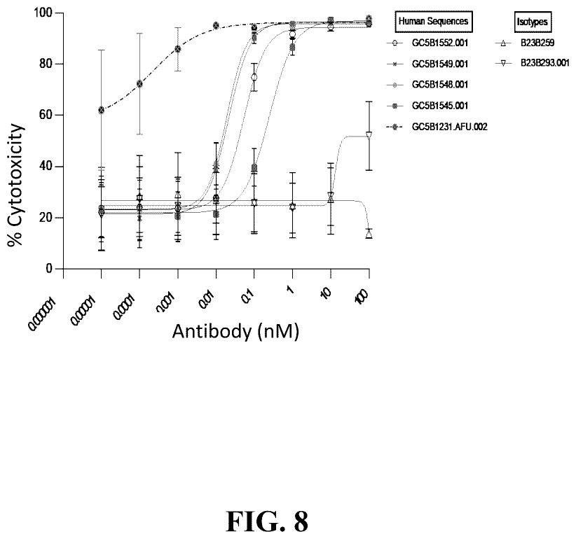

To verify ADCC activity, a set of four monovalent human anti-GPRC5D antibodies underwent assessment for ADCC-inducing activity against the MM. IR cell line. The evaluation, conducted after 48 hours using healthy donor NK cells at a 5:1 effector-to-target ratio in a flow cytometry-based assay, revealed that all four monovalent anti-GPRC5D antibodies retained varying levels of ADCC activity. This was compared to the bivalent positive control antibody, GC5B1231.AFU.002, and negative control isotypes, B23B259 (bivalent), and B23B293.001 (monovalent), as depicted in FIG. 8.

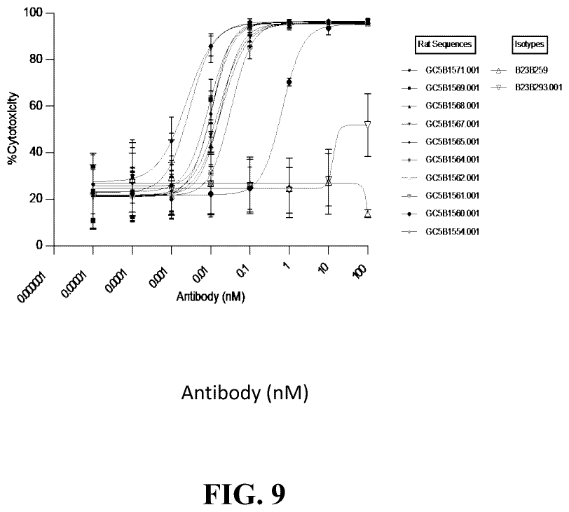

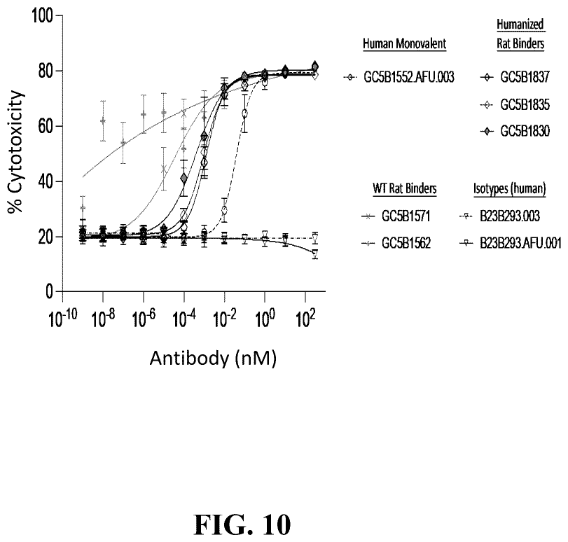

From the same panel of monovalent rat anti-human GPRC5D antibodies that had previously demonstrated CDC activity, 10 antibodies were tested for ADCC activity against the MM.1R cell line. All 10 antibodies elicited ADCC activity, as illustrated in FIG. 9. Subsequently, two of these rat anti-human GPRC5D antibodies (GC5B1571 and GC5B1562) were humanized and converted into a monovalent format, testing them for ADCC activity in comparison to the GC5B1552 antibody. The humanized anti-GPRC5D antibodies displayed increased potency in ADCC activity but demonstrated a lack of CDC and ADCC equipoise when compared to the GC5B1552 monovalent antibody, as shown in FIG. 10.

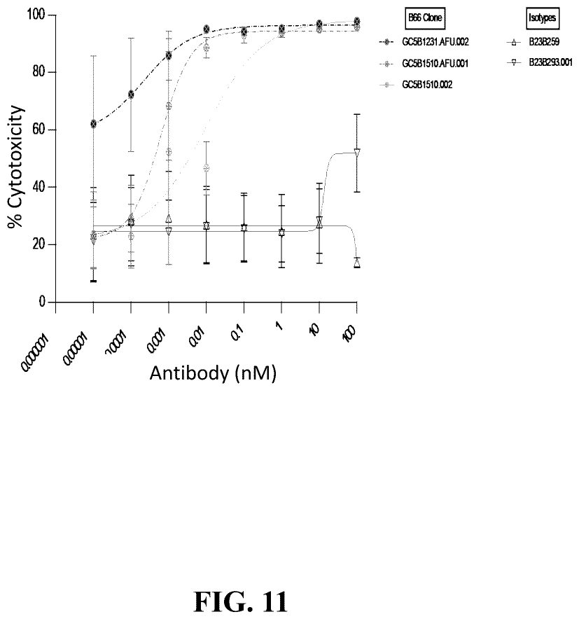

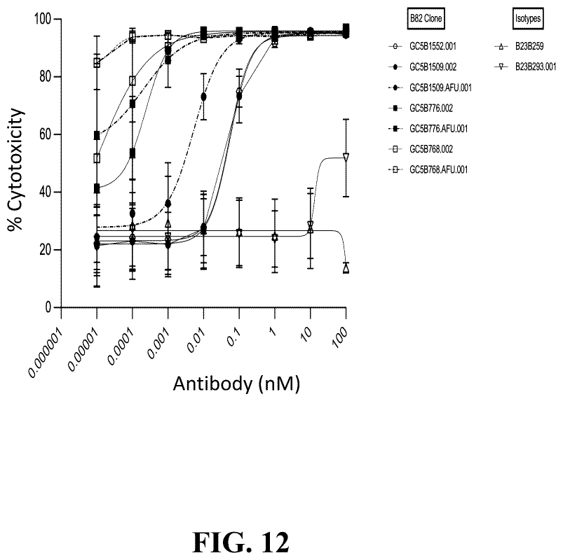

Finally, the ADCC activity of bivalent, monospecific, and monovalent anti-GPRC5D antibodies was assessed in the MM.1R cell line (FIGS. 11 and 12). While the monospecific and monovalent antibodies were less potent than the bivalent antibodies, they demonstrated better equipoise between the CDC and ADCC mechanisms.

### ADCP Evaluation:

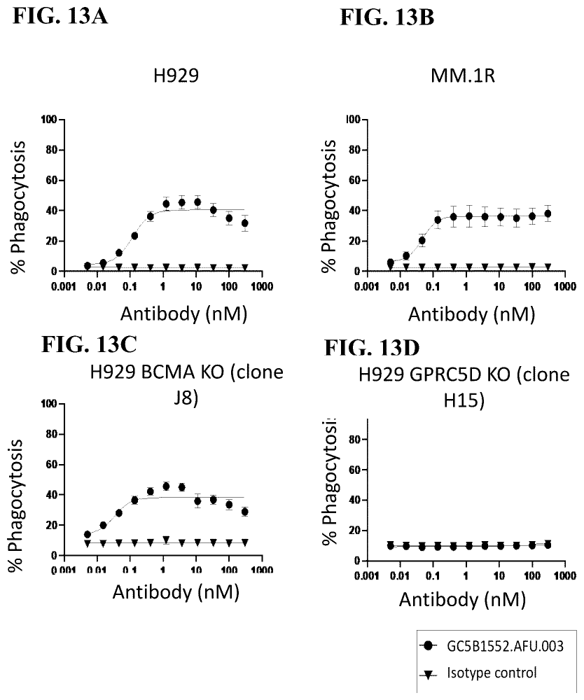

The ADCP activity of the monovalent anti-GPRC5D antibody, GC5B1552, was assessed against various cell lines, including GPRC5D+H929, H929 BCMA KO, and MM.1R, as well as the GPRC5D-, H929 GPRC5D KO cell line. GC5B1552 exhibited on-target ADCP activity against GPRC5D⁺expressing cell lines, as depicted in FIG. 13A, FIG. 13B, FIG. 13C, and FIG. 13D. However, no ADCP activity was observed against the GPRC5D-cell line.

### Example 5. Efficacy of GC5B1552.Afu.001 in Human Multiple Myeloma Xenografts with NK-92.CD16 Cell Humanized Nsg-IL15 Mice

The efficacy of GC5B1552.AFU.001 was assessed in established luciferase-transfected disseminated MM.1S or OPM-2 human multiple myeloma (MM) xenografts in female NSG-IL15 mice humanized with 10e6 NK-92.CD16 cells weekly for 3 weeks. Animals were randomly assigned to groups (n=10) based on tumor burden, determined by live bioluminescence imaging (BLI) on day 3 or 13 post-intravenous tumor implantation for MM.1S and OPM-2 xenografts, respectively. GC5B1552.AFU.001 at doses of 10, 3, and 0.3 mg/kg, or the B23B293.001 isotype control antibody at 10 mg/kg, was administered intraperitoneally weekly for 3 weeks or q3-4 days for 6 weeks, respectively. Parallel treatment groups were established without human NK-92.CD16 cells to control for mouse effector cell contributions.

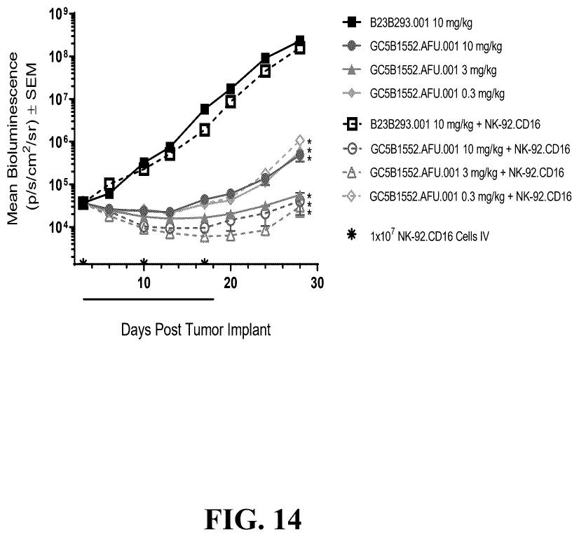

In the MM.1S xenograft model on day 28 post-tumor implantation, statistically significant (p≤0.05) antitumor efficacy was observed with GC5B1552.AFU.001 at all dose levels, resulting in 99-100% tumor growth inhibition (ATGI) with and without NK-92.CD16 cells, compared to the respective B23B293.001 control (FIG. 14). Engraftment of animals with NK-92.CD16 cells led to more partial tumor responses with GC5B1552.AFU.001 treatment at 10 or 3 mg/kg, with 8 or 7 of 10 animals, respectively, compared to 2 of 10 animals with 3 mg/kg without NK engraftment on day 28 post-tumor implantation, demonstrating NK-mediated antitumor efficacy.

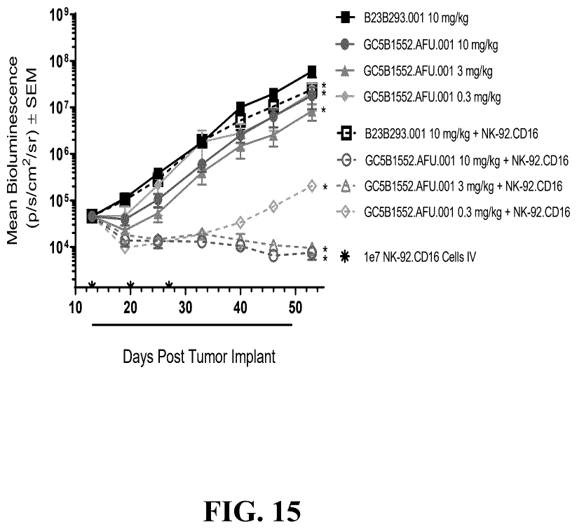

In the OPM-2 xenograft model on day 53 post-tumor implantation, statistically significant antitumor efficacy was observed with GC5B1552.AFU.001 at all concentrations with and without NK-92.CD16 cells, compared to the B23B293.001 control (FIG. 15). Treatment of animals that did not receive NK-92.CD16 cells with GC5B1552.AFU.001 at 10, 3, and 0.3 mg/kg resulted in 70%, 86%, and 65% ATGI, respectively. NK-92.CD16 engraftment enhanced antitumor activity, with treatment with GC5B1552.AFU.001 at 10, 3, and 0.3 mg/kg resulting in 100%, 100%, and 99% ATGI, respectively. In addition to enhancing TGI, NK.92.CD16 engraftment also increased the number of complete responses, with GC5B1552.AFU.001 at 10, 3, and 0.3 mg/kg resulting in 4, 3, and 4 of 10 animals, respectively, compared to no complete responses in treatment groups that did not receive NK-92.CD16 cells.

### Example 6: Affinity, Association Constant, and Dissociation Constant

Due to GPRC5D's nature as a membrane G protein-coupled receptor, the measurement of antibody affinity by Surface Plasmon Resonance (SPR) with typical recombinant antigens is not feasible. Instead, nanodiscs, synthetic model membranes, were utilized to present GPRC5D proteins in a configuration resembling their native conformation.

The binding kinetics of GPRC5D-nanodiscs (Acro, #GPD-H52D4) to anti-GPRC5D antibodies were analyzed through SPR using Biacore T200 or Biacore 8K instruments. A sensor chip surface was prepared with goat-anti-human-Fc reagent, and antibodies were captured on the sensor chip via the Fc region. GPRC5D-nanodiscs were subjected to a buffer change to running buffer using a desalting column (Thermo Fisher, #89890) following the manufacturer's recommendations. Various concentrations of GPRC5D-nanodiscs were injected onto the captured ligands, and the association (for 120 seconds) and dissociation (for 300 seconds) rates were monitored over time. The sensor chip surface was regenerated at the end of each cycle by short pulses of dilute 0.85% H3PO4 for 20 seconds (2×), followed by running buffer 1×HBSP (1×). A global fitting of all non-zero concentrations using a 1:1 Langmuir binding model was employed to determine kinetic rates (ka and kd) and binding affinity K_{D}.

Double reference subtraction was achieved by including a zero concentration of GPRC5D-nanodisc (e.g., running buffer only) and flow path correction from the reference flow cell where the capture of ligands was omitted.

TABLE 4
Binding Affinity, Association Constant, and Dissociation
Constant of GPCR5D Hits to Human GPRC5D Nanodisc

| | GPRC5D | Avg ka | Avg kd | Avg KD |
|---|---|---|---|---|
| Protein ID | Binder | (1/Ms) | (1/s) | (nM) |
| GC5B1552.AFU.003 | GP5B82 | 1.80E+04 | 1.48E−03 | 84 |
| GCDB334.001 | GC5B596 | 1.18E+04 | 4.23E−03 | 359 |
| GCDB385.003 | GC5B680 | 1.05E+04 | 3.56E−03 | 340 |
| GCDB389.002 | GP5B83 | 1.34E+04 | 2.87E−03 | 214 |
| GP5B305.002 | GP5B83 | 9.04E+03 | 2.62E−03 | 290 |
| GC5B767.002 | GP5B66 | 1.31E+04 | 1.28E−03 | 98 |

TABLE 5
Binding Affinity, Association Constant, and Dissociation
Constant of GC5B1552 to Human and Cyno GPRC5D Nanodisc

| | GPRC5D | Avg ka | Avg kd | Avg KD |
|---|---|---|---|---|
| Protein ID | Species | (1/Ms) | (1/s) | (nM) |
| GC5B1552.AFU.003 | Human | 2.20E+04 | 1.69E−03 | 77 |
| GC5B1552.AFU.003 | Cyno | 9.52E+03 | 1.79E−02 | 1881 |

### Example 7: Time Course Cell Binding

A dozen GPRC5D binders underwent evaluation for kinetic cell binding on H929-WT and MM.1R-WT cell lines. The antibodies were initially diluted in complete RPMI at a 2× concentration with an adjusted blocking mix (1:20 dilution of peptide-based block (Innovex)+125 μg/mL rat gamma globulins (Jackson)) and incubated at 37° C. in a deep well plate. Subsequently, cells were harvested and counted using Vi-Cell XR (Beckman Coulter) and added to the master deep well plate to achieve ˜1×10⁵ cells per timepoint. At each time point, cells were transferred to a round-bottom 96-well plate containing cold FACS buffer (Stain buffer containing 0.2% BSA (BD)+2 mM EDTA (ThermoFisher)), centrifuged (1200 RPM, 5 min), and the supernatant was aspirated. After two additional washes with FACS buffer, cells were stained at 4° C. with Alexa Fluor® 647 AffiniPure F (ab′)₂ Fragment Goat Anti-Human IgG, Fcγ fragment-specific diluted in Fc block (1:200, Jackson) for 30 minutes. Following incubation, cells were washed three more times with cold FACS buffer and then resuspended in FACS buffer containing Sytox Green to label all dead cells (ThermoFisher). Plates were run on the Intellicyt iQue Screener Plus (Sartorius) within three hours of staining.

Using the ForeCyt software, singlet-live populations were gated, and the median fluorescent intensity (MFI) of the APC channel was calculated for each sample. Raw data were exported to Excel (Office 365, Microsoft), where duplicates were averaged and further analyzed in Prism software (v. 9.0.0, GraphPad). Final graphs present the median fluorescent intensities of each molecule concentration at each time point.

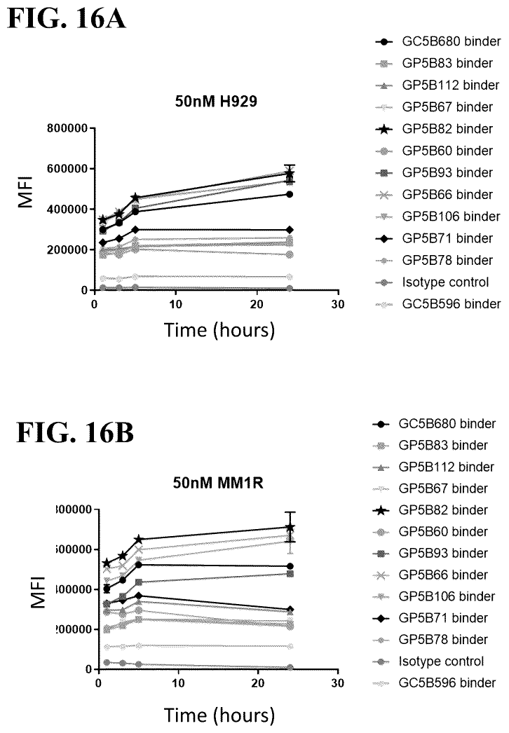

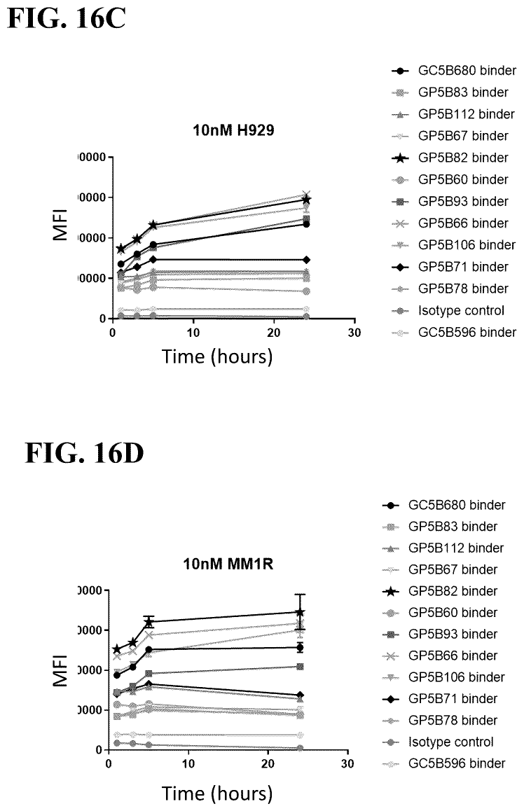

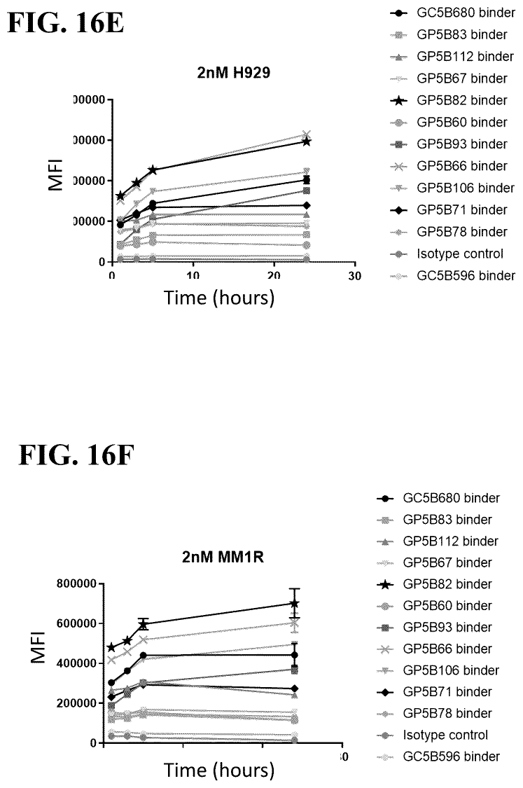

Different GPRC5D binders exhibited varying levels of binding to H929 and MMIR cells (FIG. 16A, FIG. 16B, FIG. 16C, FIG. 16D, FIG. 16E, and FIG. 16F). Notably, GP5B82 and GP5B66 binders demonstrated the strongest cell binding signal, aligning with the SPR affinity data indicating their higher affinity. The binders exhibited stable and increasing cell binding over time, suggesting no significant internalization. The kinetic binding study was repeated for GC5B1552.AFU after the final formatting conversion, and similar stable cell binding was observed.

### Example 8: Expression and Purification of Gc5B1552.Afu

GC5B1552.AFU (see amino acid and nucleic acid sequences in Tables 6 to 10) was expressed in a fucosyltransferase 8 (FUT8) knockout CHO host cell line, C3234B (Janssen R&D), to generate afucosylated antibodies. The purification process involved protein-A chromatography followed by mixed-mode column chromatography.

TABLE 6
Variable Regions and Heavy/Light Chain Sequences for GC5B1552.AFU

| | VH | | VL | | HC1 | | HC2 | | LC2 | |
|---|---|---|---|---|---|---|---|---|---|---|
| | SEQ | VH Amino | SEQ | VL Amino | SEQ | Heavy Chain | SEQ | Heavy Chain | SEQ | Light Chain |
| Clone | ID | Acid | ID | Acid | ID | 1 (HC1) | ID | 2 (HC2) | ID | 2 (LC2) |
| Name | NO | Sequence | NO: | Sequence | NO: | Sequence | NO: | Sequence | NO: | Sequence |
| GC5B | 1 | QVQLVES | 2 | DIQMTQSP | 3 | DKTHTCPPCPA | 4 | QVQLVESGGG | 5 | DIQMTQSPSSL |
| 1552. | | GGGVVQP | | SSLSASVG | | PELLGGPSVFL | | VVQPGRSLRLS | | SASVGDRVTIT |
| AFU | | GRSLRLS | | DRVTITCR | | FPPKPEDTLMI | | CAASGFTFSNY | | CRASQGIRNDL |
| | | CAASGFT | | ASQGIRND | | SRTPEVTCVV | | GMHWVRQAP | | GWYQQKPGK |
| | | FSNYGMH | | LGWYQQK | | VDVSHEDPEV | | GKGLEWVAFI | | APKRLIYAASS |
| | | WVRQAP | | PGKAPKRL | | KFNWYVDGV | | WYDGSNKYY | | LQSGVPSRFSG |
| | | GKGLEW | | IYAASSLQ | | EVHNAKTKPR | | ADSVKGRFTIS | | SGSGTEFTLTIS |
| | | VAFIWYD | | SGVPSRFS | | EEQYNSTYRV | | RDNSKNTLYL | | SLQPEDFATYY |
| | | GSNKYYA | | GSGSGTEF | | VSVLTVLHQD | | QMNSLRAEDT | | CLQRNTYPLTF |
| | | DSVKGRF | | TLTISSLQP | | WLNGKEYKC | | AVYYCAPITG | | GGGTKVEIKRT |
| | | TISRDNSK | | EDFATYYC | | KVSNKALPAPI | | YSTRWGDYW | | VAAPSVFIFPPS |
| | | NTLYLQM | | LQRNTYPL | | EKTISKAKGQP | | GQGTLVTVSS | | DEQLKSGTAS |
| | | NSLRAED | | TFGGGTKV | | REPQVYTLPPS | | ASTKGPSVFPL | | VVCLLNNFYP |
| | | TAVYYCA | | EIK | | REEMTKNQVS | | APSSKSTSGGT | | REAKVQWKV |
| | | PITGYSTR | | | | LWCLVKGFYP | | AALGCLVKDY | | DNALQSGNSQ |
| | | WGDYWG | | | | SDIAVEWESN | | FPEPVTVSWNS | | ESVTEQDSKDS |
| | | QGTLVTV | | | | GQPENNYKTT | | GALTSGVHTFP | | TYSLSSTLTLS |
| | | SS | | | | PPVLDSDGSFF | | AVLQSSGLYSL | | KADYEKHKVY |
| | | | | | | LYSKLTVDKS | | SSVVTVPSSSL | | ACEVTHQGLS |
| | | | | | | RWQQGNVFSC | | GTQTYICNVN | | SPVTKSFNRGE |
| | | | | | | SVMHEALHNH | | HKPSNTKVDK | | C |
| | | | | | | YRQKSLSLSPG | | KVEPKSCDKT | | |
| | | | | | | K | | HTCPPCPAPEL | | |
| | | | | | | | | LGGPSVFLFPP | | |
| | | | | | | | | KPEDTLMISRT | | |
| | | | | | | | | PEVTCVVVDV | | |
| | | | | | | | | SHEDPEVKFN | | |
| | | | | | | | | WYVDGVEVH | | |
| | | | | | | | | NAKTKPREEQ | | |
| | | | | | | | | YNSTYRVVSV | | |
| | | | | | | | | LTVLHQDWLN | | |
| | | | | | | | | GKEYKCKVSN | | |
| | | | | | | | | KALPAPIEKTIS | | |
| | | | | | | | | KAKGQPREPQ | | |
| | | | | | | | | VYTLPPSREEM | | |
| | | | | | | | | TKNQVSLSCA | | |
| | | | | | | | | VKGFYPSDIAV | | |
| | | | | | | | | EWESNGQPEN | | |
| | | | | | | | | NYKTTPPVLDS | | |
| | | | | | | | | DGSFFLVSKLT | | |
| | | | | | | | | VDKSRWQQG | | |
| | | | | | | | | NVFSCSVMHE | | |
| | | | | | | | | ALHNRFRQKS | | |
| | | | | | | | | LSLSPGK | | |

TABLE 7
Nucleotide sequence of GC5B1552.AFU Heavy Chain 1 gene with signal peptide
underlined.

SEQ ID NO: 27
ATGGCTAGATCCGCACTGCTCATTCTGGCTCTGCTTCTGCTTGGACTGTTCTCTCCTGGAGCATGGGG
AGACAAAACTCACACTTGTCCACCGTGCCCAGCACCTGAACTGCTGGGGGGACCGTCAGTCTTCCTC
TTCCCCCCAAAACCCGAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCC
TGCACCAGGACTGGCTGAATGGCAAGGAGTACAAGTGCAAGGTGTCCAACAAAGCCCTCCCAGCCC
CCATCGAGAAAACCATCTCCAAAGCCAAAGGGCAGCCCCGAGAACCACAGGTGTACACCCTGCCCC
CATCCCGGGAGGAGATGACCAAGAACCAGGTCAGCCTGTGGTGCCTGGTCAAAGGCTTCTATCCCA
GCGACATCGCCGTGGAGTGGGAGAGCAATGGGCAGCCGGAGAACAACTACAAGACCACGCCTCCC
GTGCTGGACTCCGACGGCTCCTTCTTCCTCTACAGCAAGCTCACCGTGGACAAGTCCAGATGGCAGC
AGGGGAACGTCTTCTCATGCTCCGTGATGCATGAGGCTCTGCACAACCACTACCGCCAGAAGTCTCT
CTCCCTGTCTCCGGGAAAA

TABLE 8
Nucleotide sequence of GC5B1552.AFU Light Chain 2 gene with signal peptide
underlined.

SEQ ID NO: 28
ATGGCCAGGAAGTCCGCTCTGCTCGCTCTGGCACTTCTGCTTCTGGGATTTGGACCTGCTTGGGCTG
ACATCCAGATGACCCAGTCTCCATCCTCTCTGTCCGCCAGCGTGGGCGACAGAGTGACCATCACCTG
TAGAGCCTCCCAGGGCATCAGAAACGACCTCGGCTGGTATCAGCAGAAGCCTGGCAAGGCCCCTAA
GCGGCTGATCTACGCTGCATCTTCTCTGCAGTCTGGCGTGCCCTCCAGATTCTCCGGATCTGGCTCTG
GCACCGAGTTTACCCTGACAATCTCCAGCCTGCAGCCTGAGGACTTCGCCACCTACTACTGCCTGCA
GCGGAACACCTATCCTCTGACCTTTGGCGGAGGCACCAAGGTGGAAATCAAGCGTACTGTGGCTGC
ACCATCTGTCTTCATCTTCCCGCCATCTGATGAGCAGTTGAAATCTGGAACTGCCTCTGTTGTGTGCC
TGCTGAATAACTTCTATCCCAGAGAGGCCAAAGTACAGTGGAAGGTGGATAACGCCCTCCAATCGG
GTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGT

TABLE 9
Nucleotide sequence of GC5B1552.AFU Heavy Chain 2 gene with signal peptide
underlined.

SEQ ID NO: 29
ATGGCCAGGAAGTCCGCTCTGCTCGCTCTGGCACTTCTGCTTCTGGGATTTGGACCTGCTTGGGCTC
AGGTGCAGCTGGTTGAATCTGGTGGCGGAGTGGTGCAGCCTGGCAGATCTCTGAGACTGTCTTGTGC
CGCCTCCGGCTTCACCTTCTCCAACTACGGAATGCACTGGGTCCGACAGGCCCCTGGCAAAGGATTG
GAGTGGGTCGCCTTCATTTGGTACGACGGCTCCAACAAGTACTACGCCGACTCCGTGAAGGGCAGA
TTCACCATCTCTCGGGACAACTCCAAGAACACCCTGTACCTGCAGATGAACTCCCTGAGAGCCGAG
GACACCGCCGTGTACTACTGTGCTCCTATCACCGGCTACTCCACCAGATGGGGCGATTATTGGGGCC
AAGGCACACTGGTCACAGTGTCCTCTGCCTCCACCAAGGGTCCATCGGTCTTCCCCCTGGCACCCTC
CTCCAAGAGCACCTCTGGGGGCACAGCGGCCCTGGGCTGCCTGGTCAAGGACTACTTCCCCGAACC
GGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCT
TGTGACAAAACTCACACTTGTCCACCGTGCCCAGCACCTGAACTGCTGGGGGGACCGTCAGTCTTCC
TCTTCCCCCCAAAACCCGAGGACACCCTCATGATCTCCCGGACCCCTGAGGTCACATGCGTGGTGGT
GGACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCAAGGTGTCCAACAAAGCCCTCCCAGC
CCCCATCGAGAAAACCATCTCCAAAGCCAAAGGGCAGCCCCGAGAACCACAGGTGTACACCCTGCC
CCCATCCCGGGAGGAGATGACCAAGAACCAGGTCAGCCTGTCCTGCGCCGTCAAAGGCTTCTATCC
CAGCGACATCGCCGTGGAGTGGGAGAGCAATGGGCAGCCGGAGAACAACTACAAGACCACGCCTC
CCGTGCTGGACTCCGACGGCTCCTTCTTCCTCGTGAGCAAGCTCACCGTGGACAAGTCCAGATGGCA
GCAGGGGAACGTCTTCTCATGCTCCGTGATGCATGAGGCTCTGCACAACAGGTTCCGCCAGAAGTCT
CTCTCCCTGTCTCCGGGAAAA

TABLE 10
List of CDR Regions for GC5B1552.AFU

| Numbering | | CDR1 | CDR2 | CDR3 |
|---|---|---|---|---|
| Format | Chain | (SEQ ID NO) | (SEQ ID NO) | (SEQ ID NO) |
| AbM | Heavy | GFTFSNYGMH (6) | FIWYDGSNKY (7) | ITGYSTRWGDY (8) |
| | Light | RASQGIRNDLG (9) | AASSLQS (10) | LQRNTYPLT (11) |
| Kabat | Heavy | NYGMH (12) | FIWYDGSNKYYADSVKG (13) | ITGYSTRWGDY (8) |
| | Light | RASQGIRNDLG (9) | AASSLQS (10) | LQRNTYPLT (11) |
| Chothia | Heavy | GFTFSNY (14) | WYDGSN (15) | ITGYSTRWGDY (8) |
| | Light | RASQGIRNDLG (9) | AASSLQS (10) | LQRNTYPLT (11) |
| IMGT | Heavy | GFTFSNYG (16) | IWYDGSNK (17) | APITGYSTRWGDY (18) |
| | Light | QGIRND (19) | AAS | LQRNTYPLT (11) |
| Contact | Heavy | SNYGMH (21) | WVAFIWYDGSNKY (22) | APITGYSTRWGD (23) |
| | Light | RNDLGWY (24) | RLIYAASSLQ (25) | LQRNTYPL (26) |

In brief, C3234B cells were cultured in suspension in ExpiCHO™ Expression Medium (ThermoFisher; Cat #A2910001) at 37° C., 8% CO2, and 125 rpm. Cells were passaged into 12×2L sterile, vented, non-baffled Erlenmeyer flasks (Corning 431255) with a 400 mL starting culture volume per flask.

On Culture Day 0, plasmid DNAs (HCl: HC2: LC chain ratio of 1:2:6) for transfection were diluted in OptiPRO medium (ThermoFisher Cat #12309019), and ExpiFectamine™ CHO transfection reagent was diluted in another portion of OptiPRO medium. The plasmid DNA and ExpiFectamine™ CHO reagent mixtures were combined and distributed across the 12 flasks. After overnight incubation (Culture Day 1), additional components were added, and cells were further incubated according to the ExpiFectamine™ CHO transfection kit Max Titer protocol.

On Culture Day 5, cells were supplemented, and incubation continued until harvest (Culture Day 12). The clarified supernatant from the transiently transfected C3234B cells was subjected to centrifugation and filtration. GC5B1552.AFU was then purified from the clarified supernatant.

The filtered cell culture supernatant was loaded onto a pre-equilibrated MabSuRe Protein A column (GE Healthcare, CV=167 mL, 4.4 cm diameter). After loading, the column was washed and eluted. The eluted protein was neutralized, pooled, and dialyzed.

Further purification was conducted by mixed-mode chromatography (MMC) using Capto MMC ImpRes resin (GE Healthcare, CV=145 mL, 3.2 cm diameter). The eluted fractions containing monomeric protein were pooled, dialyzed, and filtered.

The concentration of purified protein was determined spectrophotometrically, and its quality was assessed by SDS-PAGE and analytical SE-HPLC (Agilent HPLC system). Endotoxin levels were measured using a turbidimetric LAL assay (Pyrotell®-T, Associates of Cape Cod; Falmouth, MA).

### EXAMPLE 9: BIOPHYSICAL PROPERTIES

GPRC5D antibodies underwent assessment for various biophysical properties using the following methodologies:

1. Differential Scanning calorimetry (DSC): DSC experiments were conducted with a MicroCal Auto VP-capillary DSC system (Malvern Instruments Ltd., Malvern, UK). Samples, approximately 1.0 mg/mL in 1× PBS buffer, were subjected to a temperature ramp from 25° C. to 95° C. at a rate of 10° C./min. Analysis of resulting data was performed using MicroCal Origin 7 software.

2. NanoDSF (Differential Scanning Fluorimetry): The conformational stability of proteins was measured using advanced nanoDSF technology. Intrinsic fluorescence of tryptophan during thermal unfolding was monitored using the Prometheus NT.48 instrument (NanoTemper Technologies GmbH). Samples at 0.5 mg/ml in phosphate buffer saline (PBS) were analyzed in duplicates. Thermal melting mid-point (Tm) values and onset of aggregation (Tagg) were identified and reported.

3. CIC (Cross-Interaction Chromatography): Antibody retention times in CIC were determined by injecting 0.1 mg/mL samples into an Agilent 1200 series HPLC system with PBS as the mobile phase. Retention times were measured at 214, 254, and 280 nm using a UV detector. Peaks were visualized, and methods were described as previously published.

4. HIC (Hydrophobic Interaction Chromatography): HIC analysis was performed on an Agilent 1200 series HPLC system using a TOSOH TSKgel Butyl NPR column. Samples were injected, and an isocratic elution with 80% 2 M ammonium sulfate in PBS was carried out. The column was washed and re-equilibrated between runs.

5. SEC (Size Exclusion Chromatography): For each SEC experiment, the antibody was coupled to Toyopearl AF-tresyl-650M chromatography resin. Peaks were visualized at 215 nm, and retention times were determined using HPLC Ettan LC system UNICORN software.

6. Viscosity Measurement: Viscosity of the sample at 100 mg/mL was determined as a function of shear rate using the Neo Visc Multiplexed Viscometer (Neofluidics LLC, California, USA). Measurements were conducted at 25° C., and viscosity values were reported as averages of duplicates.

TABLE 11
Biophysical Assessments of GPRC5D Binders

| | | Conformational Stability by | | | | Hydrophobicity | | |
|---|---|---|---|---|---|---|---|---|
| | | Nano-DSF | | | | by aHIC | | CIC |
| | | Tonset | Tm1 | Tm2 | Tagg | Rt | Hydrophobicity | Rt |
| ID | Binder | (° C.) | (° C.) | (° C.) | (° C.) | (min) | index | (min) |
| GC5B751 | GC5B680 | 60.5 | 68.3 | 78.6 | 85.9 | 3.07 | 0.53 | 4.82 |
| GC5B752 | GP5B83 | 61.6 | 68.3 | 79.0 | 83.4 | 4.03 | 0.70 | 5.52 |
| GC5B754 | GP5B66 | 63.0 | Unresolved | 74.7 | 73.5 | 3.02 | 0.54 | 4.95 |
| GC5B776 | GP5B82 | 57.6 | 63.2 | 79.4 | 65.2 | 3.16 | 0.57 | 5.62 |

Out of the GPRC5D antibodies assessed for biophysical properties, GC5B776 (bivalent format of GC5B1552) exhibits the overall most favorable profile, indicating minimal manufacturing developability risks (Table 11). For instance, GC5B752 demonstrates a higher retention time and hydrophobicity index, while GC5B754 exhibits higher viscosity at 22.6 cP at 90 mg/mL in 10 mM Histidine pH 6.5, compared to 4.6 cP for GC5B 1552 at 100 mg/mL in Acetate pH 5.5.

GC5B1552 was measured in duplicates to have Tonset=57.6° C., Tml=65.2° C. and Tm2 =68.9° C. Additionally, GC5B 1552 displayed a retention time of 5.40 minutes and a peak width of 1.06 minutes. Comparative analysis revealed that GC5B1552 demonstrates lower hydrophobicity (Hydrophobicity index, HI=0.42) in contrast to a well-established control monoclonal antibody, CNTO5825 (HI=0.49).

### EXAMPLE 10: FCγR AFFINITY AND KINETICS

The assessment of FcγR-antibody interactions was conducted using Surface Plasmon Resonance (SPR) on a four-channel Biacore T200 optical biosensor system at 25° C. (GE Healthcare, Piscataway, NJ). To prepare the biosensor surface, anti-his antibody was coupled to a CM4 sensor chip using amine-coupling chemistry. Following activation and deactivation steps, the Fcγ receptors (FcgRI, FcgRIIa, FcgRIIb, FcgRIII) from human and cynomolgus sources were captured on separate flow cells, with flow cell 1 serving as a reference.

Optimal capture levels for each receptor were determined, and subsequent interaction cycles assessed the binding of antibodies (GC5B1552.AFU.003 and CNTO3930) to each Fcγ receptor. After injection, the dissociation phase was monitored, and the sensor chip surface was regenerated for subsequent cycles.

The antibody titration series for each Fcγ receptor included a range of concentrations, and interaction parameters were set accordingly. Sensorgrams were processed and analyzed using Biacore T200 Evaluation software. Double-referenced sensorgrams were fitted to a 1:1 Langmuir binding model for CD64 and CD16 receptors or an Equilibrium Steady-State binding model for CD32 receptors to determine binding kinetics and affinities.

The results for FcγR-antibody interactions were reported as an average of triplicates, presenting kon (on-rate), k_{off} (off-rate), and KD (equilibrium dissociation constant), or KD only for steady-state analysis.

GC5B1552.AFU exhibits binding to both human and cynomolgus FcγRs, as summarized in Table 12. Notably, it demonstrates a 50.7- and 111.3-fold increase in binding affinities to human FcγRIIIa 158F and V alleles, respectively. This heightened binding to FcγRIIIa contributes significantly to the enhancement of ADCC mechanisms.

TABLE 12
FcγR-antibody affinity and kinetics

| | | | | | 95% | | 95% | Fold |
|---|---|---|---|---|---|---|---|---|
| | | Avg | 95% CI | Avg | CI | Avg | CI | increase |
| Description | Receptor | ka (1/Ms) | | kd (1/s) | | KD (nM) | | in binding |
| wt IgG1 control | cy-RI | 3.70E+05 | 4.77E+04 | 5.66E−04 | 5.82E−05 | 1.5 | 0.1 | 1 |
| | cy-RIIa | N/A | | N/A | | 8150 | 311 | 1 |
| | cy-RIIb | N/A | | N/A | | 4453 | 100 | 1 |
| | cy-RIII | 3.92E+05 | 5.25E+04 | 1.55E−01 | 9.40E−03 | 395 | 55 | 1 |
| | hu-RI | 2.54E+05 | 2.96E+04 | 7.24E−04 | 9.17E−05 | 2.9 | 0.6 | 1 |
| | hu-RIIa | N/A | | N/A | | 9333 | 3667 | 1 |
| | hu-RIIb/c | N/A | | N/A | | 11200 | 1551 | 1 |
| | Hu-RIIIaF | 7.92E+04 | 9.61E+03 | 2.99E−01 | 3.11E−02 | 3780 | 618 | 1 |
| | Hu-RIIIa V | 2.40E+05 | 2.99E+04 | 2.17E−01 | 8.97E−02 | 905 | 449 | 1 |
| GC5B1552.AFU | cy-RI | 6.47E+05 | 5.97E+04 | 6.59E−04 | 4.45E−05 | 1.0 | 0.1 | 1.5 |
| | cy-RIIa | N/A | | N/A | | 8817 | 1607 | 0.9 |
| | cy-RIIb | N/A | | N/A | | 5400 | 358 | 0.8 |
| | cy-RIII | 2.86E+06 | 3.19E+05 | 3.40E−02 | 2.15E−03 | 11.9 | 1.1 | 33.2 |
| | hu-RI | 5.67E+05 | 6.67E+04 | 7.38E−04 | 1.08E−04 | 1.3 | 0.3 | 2.2 |
| | hu-RIIa | N/A | | N/A | | 3707 | 1133 | 2.5 |
| | hu-RIIb/c | N/A | | N/A | | 11600 | 3608 | 1.0 |
| | Hu-RIIIaF | 2.24E+06 | 1.63E+06 | 1.67E−01 | 1.18E−01 | 75 | 1 | 50.7 |
| | Hu-RIIIaV | 2.85E+06 | 1.88E+05 | 2.32E−02 | 1.14E−03 | 8.1 | 0.6 | 111.3 |

### EXAMPLE 11: CYNOMOLGUS MONKEY PHARMACOKINETICS

The pharmacokinetic (PK) characteristics of GC5B1552.AFU and GC5B1509.AFU were evaluated in a single-dose, non-GLP PK study intravenously (IV) administered to female and male cynomolgus monkeys, with three animals per molecule. The IV administration was conducted at a dose of 0.5 mg/kg (in 10 mM histidine, pH 6.0). Total GC5B1552.AFU concentrations were quantified using a qualified electrochemiluminescence immunoassay (ECLIA) from Meso Scale Discovery (MSD).

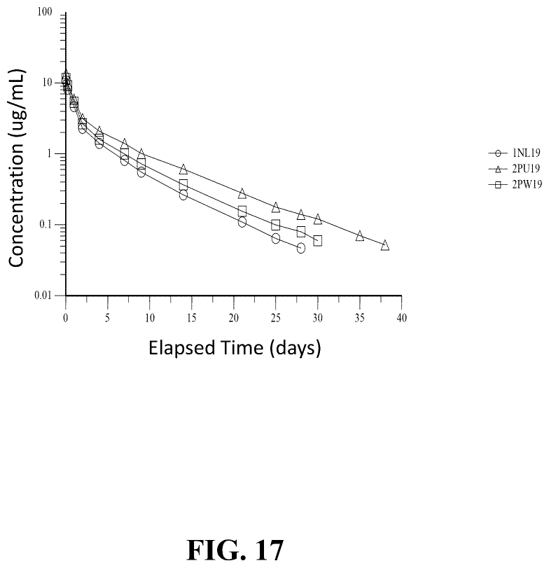

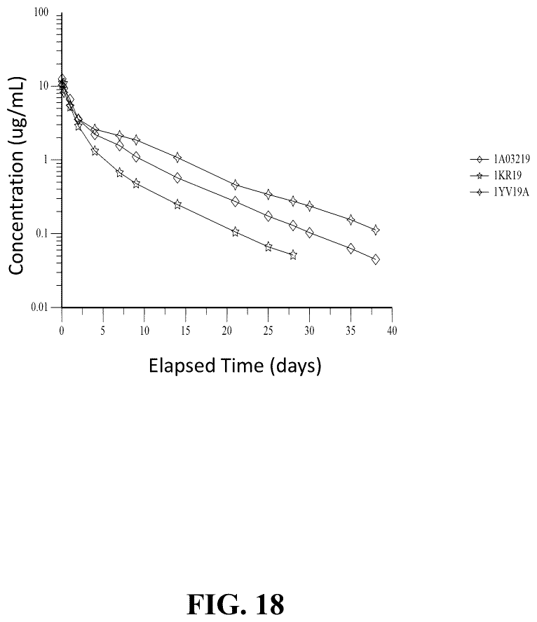

Systemic drug exposure was assessed by determining the maximum serum drug concentration (Cmax) and the area under the drug serum concentration-time curve (AUC) for one dose interval, as presented in Table 13. Serum concentrations over time are depicted in FIGS. 17 and 18.

The heavy chains of GC5B1552.AFU incorporate mutations (K248E and T437R) in the constant region (Fc), representing a novel and crucial design aimed at enhancing complement-dependent cytotoxicity. Notably, the PK properties and serum concentrations of GC5B1552.AFU closely resemble those of GC5B1509.AFU, which shares the same binder and monovalency but lacks the K248E and T437R mutations.

TABLE 13
Individual and Mean (SD) Serum Pharmacokinetic Estimates Following
a Single IV Dose of 0.5 mg/kg in Cynomolgus Monkeys

| | | | Cₘₐₓ | AUCₗₐₛₜ | AUC_{inf} | V_{z} | CL | T_{1/2} |
|---|---|---|---|---|---|---|---|---|
| Group | Gender | ID | (μg/mL) | (μg · day/mL) | (μg · day/mL) | (mL/kg) | (mL/day/kg) | (day) |
| GROUP 1 | Male | 1NL19 | 10.66 | 22.29 | 22.65 | 176.72 | 22.07 | 5.55 |
| GC5B1552.AFU | Female | 2PU19 | 13.48 | 35.03 | 35.52 | 133.36 | 14.08 | 6.57 |
| | Female | 2PW19 | 11.47 | 26.72 | 27.20 | 152.79 | 18.39 | 5.76 |
| | | Mean | 11.87 | 28.01 | 28.45 | 154.29 | 18.18 | 5.96 |
| | | SD | 1.45 | 6.47 | 6.52 | 21.72 | 4.00 | 0.54 |
| GROUP 2 | Female | 1A03219 | 12.54 | 36.66 | 37.09 | 127.65 | 13.48 | 6.56 |
| GC5B1509.AFU | Male | 1KR19 | 11.06 | 24.06 | 24.47 | 172.13 | 20.43 | 5.84 |
| | Male | 1YV19A | 10.42 | 45.89 | 47.02 | 111.34 | 10.63 | 7.26 |
| | | Mean | 11.34 | 35.54 | 36.19 | 137.04 | 14.85 | 6.55 |
| | | SD | 1.09 | 10.96 | 11.30 | 31.46 | 5.04 | 0.71 |

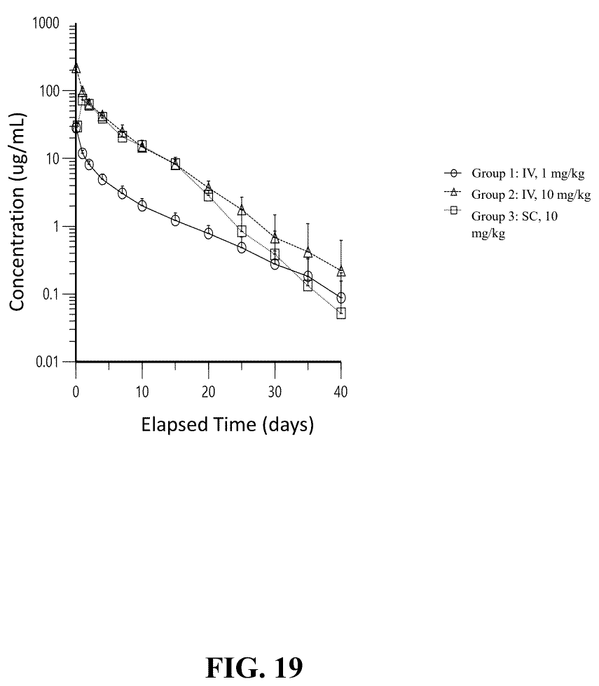

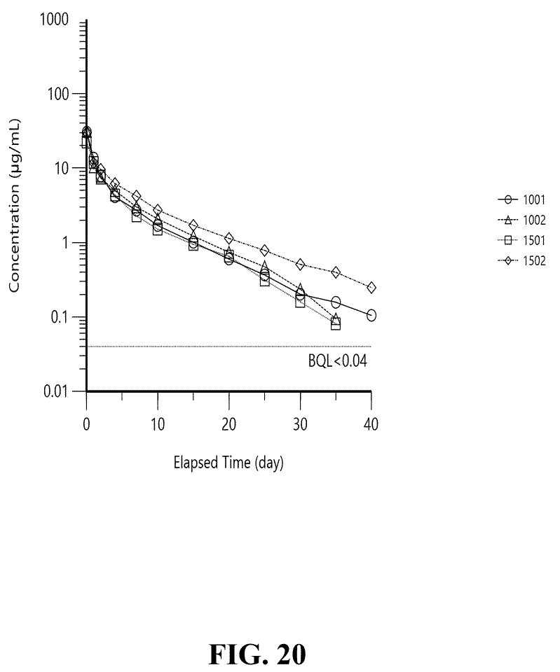

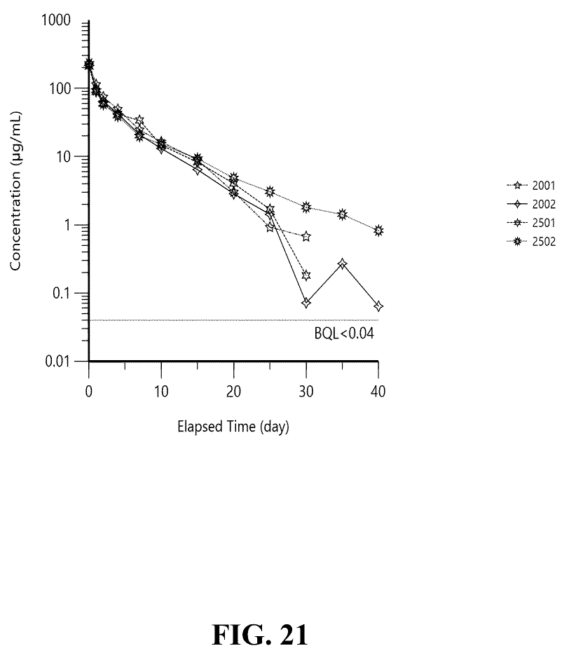

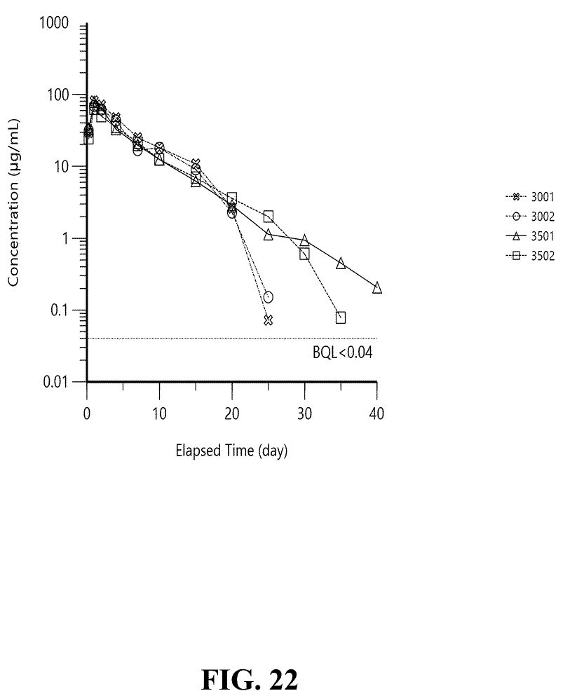

The pharmacokinetic (PK) characteristics and bioavailability of GC5B1231.AFU (a bivalent GPRC5D mAb) were evaluated in a distinct study involving 12 cynomolgus monkeys, each receiving a single dose of GC5B1231.AFU. The initial dosing occurred on Day 1, and the study design is outlined in Table 13. Quantification of GC5B1231.AFU concentrations was performed using a validated Electrochemiluminescence Immunoassay (ECLIA) method on the MSD platform. The summarized PK parameter estimates are presented in Table 15, with serum concentrations depicted in FIGS. 19-22.

GC5B1231.AFU contains the GPRC5D binder GP5B66. Another antibody, GC5B754, containing the same binder, exhibited notably high viscosity at 22.6 cP when at 90 mg/mL. In contrast, GC5B1552.AFU displayed a significantly lower viscosity of 4.6 cP at 100 mg/mL, indicating superior biophysical characteristics compared to GC5B754. Given the known impact of suboptimal biophysical traits on the pharmacokinetic (PK) properties of therapeutic antibodies, GC5B1552.AFU was selected due to its superior biophysical attributes. This choice aims to potentially mitigate both the risks associated with chemistry, manufacturing, and control (CMC), as well as PK concerns in future developments.

TABLE 14
PK study design of GC5B1231.AFU

| | | Dose | | |
|---|---|---|---|---|
| Group | Test Article | (mg/kg) | Route | Number of Animals |
| 1 | GC5B1231.AFU | 1 | IV | 2Males and 2Females |
| 2 | GC5B1231.AFU | 10 | IV | 2Males and 2Females |
| 3 | GC5B1231.AFU | 10 | SC | 2Males and 2Females |

TABLE 15
Individual and Mean (SD) Serum GC5B1552.AFU PK Parameter Estimates
Following a Single Dose of GC5B1552.AFU in Cynomolgus Monkeys

| | | Cₘₐₓ | Tₘₐₓ | AUCₗₐₛₜ | AUC_{inf} | C_{L} | V_{z} | T_{1/2} |
|---|---|---|---|---|---|---|---|---|
| Group | ID | (μg/mL) | (day) | (μg · day/mL) | (μg · day/mL) | (mL/day/kg) | (mL/kg) | (day) |
| Group 1 | 1001 | 30.31 | — | 75.81 | 77.10 | 12.97 | 162.29 | 8.67 |
| IV | 1002 | 31.08 | — | 78.02 | 78.80 | 12.69 | 92.40 | 5.05 |
| 1 mg/kg | 1501 | 22.27 | — | 67.46 | 68.03 | 14.70 | 105.89 | 4.99 |
| | 1502 | 30.23 | — | 101.68 | 105.14 | 9.51 | 128.73 | 9.38 |
| | Mean | 28.47 | — | 80.74 | 82.27 | 12.47 | 122.33 | 7.02 |
| | SD | 4.15 | — | 14.68 | 15.96 | 2.16 | 30.57 | 2.33 |
| Group 2 | 2001 | 229.96 | — | 647.19 | 652.57 | 15.32 | 78.78 | 3.56 |
| IV | 2002 | 214.93 | — | 557.73 | 567.10 | 17.63 | 117.95 | 4.64 |
| 10 mg/kg | 2501 | 209.22 | — | 597.78 | 610.42 | 16.38 | 113.71 | 4.81 |
| | 2502 | 232.07 | — | 602.96 | 613.13 | 16.31 | 195.17 | 8.29 |
| | Mean | 221.55 | — | 601.41 | 610.81 | 16.41 | 126.41 | 5.33 |
| | SD | 11.21 | — | 36.60 | 34.93 | 0.95 | 49.09 | 2.05 |
| Group 3 | 3001 | 81.66 | 1.00 | 509.89 | 529.27 | — | — | 4.10 |
| SC | 3002 | 69.35 | 1.00 | 438.37 | 450.78 | — | — | 3.35 |
| 10 mg/kg | 3501 | 79.04 | 1.00 | 422.48 | 424.31 | — | — | 5.60 |
| | 3502 | 63.47 | 1.00 | 395.84 | 411.95 | — | — | 5.63 |
| | Mean | 73.38 | 1.00 | 441.64 | 454.07 | — | — | 4.67 |
| | SD | 8.47 | 0.00 | 48.77 | 52.68 | — | — | 1.13 |

### EXAMPLE 10: OFF-TARGET BINDING SCREEN

To evaluate non-specific binding, antibodies featuring diverse GPRC5D binders underwent scrutiny using Retrogenix cell microarray technology. The screening library encompassed over 5000 expression vectors, encoding full-length human plasma membrane proteins, secreted proteins, or cell surface-tethered secreted proteins. Each vector included ZsGreen 1 for transfection confirmation and was arrayed in duplicate across multiple microarray slides. Quadruplicate spots of an expression vector (pIRES-hEGFR-IRES-ZsGreen1) were included on every slide to ensure a threshold of transfection efficiency. Reverse transfection/expression was performed in human HEK293 cells, followed by the addition of GPRC5D antibodies at a determined final concentration, typically 2 μg/mL, post-cell fixation. Binding detection utilized an AlexaFluor647-labeled anti-human IgG Fc detection antibody, validated in the pre-screen. Image analysis and quantification, including transfection efficiency, were conducted using ImageQuant software (GE Healthcare, Version 8.2). A protein ‘hit’ was identified when duplicate spots exhibited an elevated signal compared to background levels, categorized as ‘strong, medium, weak, or very weak’ based on intensity. All GPRC5D antibodies demonstrated strong binding to the primary target GPRC5D in this microarray. However, potential non-specific interactions were observed for certain binders. Further investigation of these hits was carried out using flow cytometry. Live HEK293 cells were transfected with vectors encoding the hits and ZsGreen1-only (negative control), followed by incubation with 1 or 2 μg/mL GPRC5D antibodies. Positive and negative assay controls were included, and assessments were made using flow cytometry. In the flow cytometry confirmation, some GPRC5D antibodies exhibited weak non-specific interactions, albeit significantly weaker than the primary interaction with GPRC5D. Conversely, GC5B1552.AFU exhibited robust binding exclusively with GPRC5D, showcasing high specificity for its primary target. These results underscore the specificity of GC5B1552.AFU for GPRC5D.

### EXAMPLE 11: CHARACTERIZATION OF GC5B1552.AFU IN HUMAN WHOLE BLOOD OR PBMC CO-CULTURES WITH JIM-3 CELLS

### PBMC Co-Culture Assay

JIM-3 cells were harvested by centrifugation at 1,500 rpm for 3 minutes at RT and washed once in DPBS. For every 1×10⁶ viable JIM-3 cells, 1 mL of diluted 5-carboxyfluorescein succinimidyl ester (CFSE) dye (reconstituted in 18 μL dimethyl sulfoxide [DMSO] and diluted 1:10,000 in DPBS) was added and incubated for 8 minutes at RT, protected from light. The staining reaction was quenched by adding 1 mL of heat inactivated fetal bovine serum, then washed with complete culture medium. JIM-3 cells were counted using the Vi-CELL XR Cell Viability Analyzer (Beckman Coulter) utilizing Trypan Blue exclusion, adjusted to 0.11×10⁶ viable cells/mL in complete medium, and 90 μL/well was added to a 96-well round bottom plate, yielding 1.0×10⁴ viable JIM-3 cells/well.

PBMCs were thawed, harvested, and diluted to a final concentration of 3.3×10⁶ viable cells/mL in complete culture medium, to deliver 3×10⁵ viable cells/well in 90 μL, which was added to the assay plate containing JIM-3 target cells resulting in a total PBMC to target (PBMC: T) ratio of 30:1 and an approximate NK to target (NK: T) ratio of 5:1.

After the addition of PBMCs and JIM-3 target cells, plates were centrifuged at 1,500 rpm for 5 minutes at RT. Following centrifugation, 80 μL of the supernatant was carefully removed and discarded, and 80 μL/well of commercial pooled normal human serum complement (NHSC) was added to yield a final serum concentration in the well of 40%. This step was done to specific plates where the CDC mechanism was introduced into the co-culture assay with PBMCs and JIM-3 cells.

GC5B1552.AFU and the isotype control were prepared at a 10× concentration of 3 uM, 0.5 uM, and 0.1 uM in complete medium, then 20 μL/well was added to deliver final concentrations in the well of 300 nM, 50 nM, and 10 nM. As a positive control for effector activation independent of therapeutic, Cell Stimulation Cocktail containing phorbol 12-myristate 13-acetate (PMA) and ionomycin was added into the positive control wells to yield a final concentration of 2 μg/mL in the well. The plates were incubated for 24 and 48 hours at 37° C., 5% CO₂.

After incubation, plates were centrifuged at 1,500 rpm for 5 minutes at RT, and 100 μL/well of supernatant was collected, and stored at −80° C. for cytokine and chemokine analysis. Samples were stained and acquired on a BD FACSymphony Al flow cytometer (BD Biosciences). Samples were analyzed in FACSDiva software (BD Biosciences, Version 9.0.2) by using the following gating strategy: FSC×SSC to identify the total cell population, followed by CFSE⁻ or CFSE⁺to identify effector cell and tumor cell populations, respectively. Dead tumor target cells were identified as LIVE/DEAD Near-IR⁺ from the CFSE⁺parent population. Live effector cells were identified as LIVE/DEAD Near-IR from the CFSE parent population. Subsequently, NK cells were identified as CD56⁺ and activated NK cells were then identified as either CD25⁺ or CD137⁺.

Percent target cell cytotoxicity or NK-cell activation (expressed as CD25⁺ or CD137⁺NK cells) were graphed with GraphPad Prism (Version 9.5.1) using a non-linear regression two-way analysis of variance (ANOVA) mixed model. For statistical analysis, cytotoxicity and activation were analyzed by comparing GC5B1552.AFU and the vehicle control at each concentration via a t-test, assuming unequal variance was used to compare the means of the logarithm of the responses. P values were adjusted for multiple comparisons within an endpoint using the false discovery rate method, and all analyses were performed in R (Version 4.3.0).

### Whole-Blood Co-Culture Assay

A total of 100 μL/well of healthy donor whole blood was added to a 96-well round bottom plate. Based on the NK cell counts determined in 100 μL of healthy donor whole blood, a fixed number of JIM-3 cells was plated for all conditions. JIM-3 cells were harvested and labelled with CFSE and adjusted to 0.2×10⁶ viable cells/mL in complete culture medium, and 50 μL/well was added to a 96-well round bottom plate, yielding 1.0×10⁴ viable JIM-3 cells/well. This resulted in a final NK: T cell ratio of 1-1.5:1 which is lower than standard in vitro ADCC assays, where a 5:1 ratio is normally used.

GC5B1552.AFU, isotype control and positive controls were prepared and added to the plates. Complete medium was then added appropriately to achieve a final volume of 200 μL/well. Plates were incubated at 37° C., 5% CO₂ for 24 and 48 hours.

After incubation, plates were centrifuged at 1,500 rpm for 5 minutes at RT, and 100 L/well of supernatant was collected and stored at −80° C. for cytokine and chemokine analysis. Plates were processed and stained. Samples were acquired on a BD FACSymphony Al flow cytometer, and raw data files were analyzed using FlowJo (Version 10.9.0). Untreated, live NK-cell and JIM-3 cell counts were used to determine final NK: T ratios in the well for each timepoint.

Percent target cell cytotoxicity or NK-cell activation (expressed as CD25⁺ and CD137+NK cells) were graphed with GraphPad Prism (Version 9.5.1) using a non-linear regression two-way ANOVA mixed model. For statistical analysis, cytotoxicity and activation were analyzed by comparing GC5B1552.AFU and the vehicle control at each concentration via a t-test, assuming unequal variance was used to compare the means of the logarithm of the responses. P-values were adjusted for multiple comparisons within an endpoint using the false discovery rate method, and all analyses were performed in R (Version 4.3.0).

### Cytokine/Chemokine Detection Protocol

GC5B1552.AFU-induced production of IFN-γ, IL-1B, IL-2, IL-6, IL-10, TNF-α, MCP-1, MIP-1a, MIP-1B and IL-1RA in PBMC or whole blood coculture assays was assessed using the Meso Scale Discovery (MSD) custom U-PLEX human biomarker multiplex kit.

Supernatants (100 μL/well) were removed from all assay plates after 24 hours, stored at −80° C. and then thawed at RT for 30 minutes. Samples were assayed following the manufacturer's recommendations and plates were scanned on a Sector 6000 Imager (MSD). Analysis of data and extrapolation of samples from the standard curve was done using DISCOVERY WORKBENCH (MSD, Version 4.0.13).

Data was graphed using a non-linear regression two-way ANOVA mixed model in GraphPad Prism (Version 9.5.1). For statistical analysis, secreted cytokine and chemokine concentrations were analyzed by comparing GC5B1552.AFU and the vehicle control at each concentration via a t-test, assuming unequal variance was used to compare the means of the logarithm of the responses. P-values were adjusted for multiple comparisons within an endpoint using the false discovery rate method, and all analyses were performed in R (Version 4.3.0).

### RESULTS

### Effect of GC5B1552.AFU in PBMC Coculture Assays Using JIM-3 Cells

Cytotoxicity and NK-cell Activation in PBMC Cocultures

The functional activity of GC5B1552.AFU was assessed in healthy donor PBMC assays cocultured with JIM-3 cells in the absence or presence of 40% NHSC after 24 and 48 hours.

GC5B1552.AFU induced significant (p<0.05) cytotoxicity of JIM-3 cells in the absence or presence of NHSC when compared to vehicle control at 24 hours (see Table 16). Furthermore, the presence of 40% NHSC resulted in more potent cytotoxicity of JIM-3 cells than the absence of NHSC. Mean and range values of cytotoxicity for GC5B1552.AFU and vehicle control are displayed in Table 17. There were no significant (p<0.05) increases in mean NK-cell activation observed in any of the conditions tested (see Table 18).

TABLE 16
Cytotoxicity and NK-Cell Activation P-Values Observed
for GC5B1552.AFU in the Presence or Absence of NHSC
in PBMC Coculture Assays Using JIM-3 Target Cells.

| | | | |
|---|---|---|---|
| Time (hrs) | 300 nM | 50 nM | 10 nM |

PBMCs + JIM-3 Cells
Cytotoxicity

| | | | |
|---|---|---|---|
| 24 | 0.034* | 0.035* | 0.044* |
| 48 | 0.171 | 0.148 | 0.148 |

PBMCs + JIM-3 Cells + 40% Normal Human Serum Complement
Cytotoxicity

| | | | |
|---|---|---|---|
| 24 | 0.003** | 0.039* | 0.084 |
| 48 | 0.214 | 0.44 | 0.518 |

CD, cluster of differentiation; GPRC5D, G-protein-coupled receptor Family C Group 5 Member D; hrs, hours; NHSC, normal human serum complement; NK, natural killer; PBMC, peripheral blood mononuclear cell; PBMC:T, peripheral blood mononuclear cell to target.
P-values for PBMC coculture assays using PBMCs from 5 healthy donors to measure cytotoxicity and NK cell activation (expressed as CD25⁺ and CD137⁺ NK cells) with GC5B1552.AFU and GPRC5D⁺ JIM-3 cells, at a PBMC:T-cell ratio of 30:1 in the presence or absence of 40% NHSC for 24 and 48 hours. A t-test was used to compare the means of the logarithm of the responses.
*indicates p-values < 0.05,
**indicates p-value < 0.01 between GC5B1552.AFU and vehicle control.

TABLE 17
Mean Percent Cytotoxicity and NK-Cell Activation Values Observed
for GC5B1552.AFU and Vehicle Control in the Presence or Absence
of NHSC in PBMC Coculture Assays of JIM-3 Target Cells.

| Time | GC5B1552.AFU | | | Vehicle Control |
|---|---|---|---|---|
| (hrs) | 300 nM | 50 nM | 10 nM | 0 nM |

PBMCs + JIM-3 Cells
Cytotoxicity

| | | | | |
|---|---|---|---|---|
| 24 | 28.4, [23.0, 34.8] | 30.1, [22.3, 39.6] | 29.3, [22.4, 42.2] | 20.8, [17.8, 23.7] |
| 48 | 46.5, [27.3, 82.7] | 46.7, [25.7, 66.9] | 46.5, [26.9, 66.9] | 28.7, [18.1, 46.1] |

PBMCs + JIM-3 Cells + NHSC
Cytotoxicity

| | | | | |
|---|---|---|---|---|
| 24 | 43.1, [29.0, 60.1] | 35.6, [17.9, 52.2] | 25.9, [15.9, 35.7] | 17.0, [10.8, 23.0] |
| 48 | 34.5, [19.1, 52.3] | 26.4, [14.8, 61.6] | 23.2, [11.1, 56.1] | 18.4, [6.90, 52.8] |

PBMCs + JIM-3 Cells CD137⁺
NK-Cell Activation

| | | | | |
|---|---|---|---|---|
| 24 | 11.1, [5.70, 19.8] | 9.04, [5.60, 13.0] | 8.48, [5.00, 13.2] | 6.02, [4.55, 7.20] |
| 48 | 6.88, [3.60, 11.0] | 6.28, [3.60, 9.20] | 5.62, [3.60, 7.50] | 4.23, [2.90, 5.70] |

PBMCs + JIM-3 Cells + NHSC CD137⁺
NK-Cell Activation

| | | | | |
|---|---|---|---|---|
| 24 | 8.14, [3.40, 19.9] | 8.20, [3.40, 18.8] | 9.32, [3.20, 22.1] | 6.53, [3.70, 12.8] |
| 48 | 4.26, [1.70, 6.90] | 4.28, [1.40, 7.30] | 4.34, [2.40, 6.50] | 3.86, [1.80, 6.55] |

PBMCs + JIM-3 Cells CD25⁺
NK-Cell Activation

| | | | | |
|---|---|---|---|---|
| 24 | 18.0, [8.40, 27.6] | 17.5, [8.80, 26.1] | 15.4, [8.20, 23.4] | 14.2, [7.10, 20.0] |
| 48 | 15.1, [6.80, 22.9] | 16.2, [7.80, 24.8] | 14.9, [7.90, 21.7] | 13.0, [6.70, 18.2] |

PBMCs + JIM-3 Cells + NHSC CD25⁺
NK-Cell Activation

| | | | | |
|---|---|---|---|---|
| 24 | 21.5, [8.80, 34.1] | 20.6, [9.30, 31.6] | 20.4, [7.10, 34.3] | 18.6, [7.95, 27.7] |
| 48 | 21.4, [11.0, 26.7] | 22.9, [10.7, 29.6] | 22.6, [11.2, 31.0] | 20.9, [8.50, 28.6] |

CD, cluster of differentiation; hrs, hours; GPRC5D, G-protein-coupled receptor Family C Group 5 Member D; NHSC, normal human serum complement; NK, natural killer; PBMC, peripheral blood mononuclear cell PBMC:T, peripheral blood mononuclear cell to target.
Percent cytotoxicity and NK-cell activation (expressed as CD25⁺ and CD137⁺ NK cells) values for GC5B1552.AFU and vehicle control in the absence or presence of 40% normal human serum complement in PBMC coculture assays

TABLE 18
Individual Donor Cytotoxicity and NK-cell Activation Fold
Change Values Observed for GC5B1552.AFU Relative to Vehicle
Control in the Presence or Absence of NHSC in PBMC Cytotoxicity
Assays Using JIM-3 Target Cells.

| | | | | | |
|---|---|---|---|---|---|
| | Donor | Donor | Donor | Donor | Donor |
| GC5B1552.AFU | ID# | ID# | ID# | ID# | ID# |

PBMCs + JIM-3 Cells

| | | | | | | |
|---|---|---|---|---|---|---|
| 300 | nM | 1 | 1 | 2 | 1 | 1 |
| 50 | nM | 1 | 1 | 2 | 1 | 1 |
| 10 | nM | 1 | 1 | 2 | 1 | 2 |

PBMCs + JIM-3 Cells

| | | | | | | |
|---|---|---|---|---|---|---|
| 300 | nM | 1 | 2 | 2 | 1 | 1 |
| 50 | nM | 1 | 2 | 2 | 2 | 1 |
| 10 | nM | 1 | 2 | 2 | 2 | 1 |

PBMCs + JIM-3 Cells + NHSC

| | | | | | | |
|---|---|---|---|---|---|---|
| 300 | nM | 3 | 4 | 2 | 3 | 2 |
| 50 | nM | 2 | 3 | 2 | 2 | 2 |
| 10 | nM | 2 | 1 | 1 | 1 | 2 |

PBMCs + JIM-3 Cells + NHSC

| | | | | | | |
|---|---|---|---|---|---|---|
| 300 | nM | 4 | 5 | 2 | 2 | 1 |
| 50 | nM | 3 | 2 | 1 | 2 | 1 |
| 10 | nM | 1 | 2 | 2 | 1 | 1 |

PBMCs + JIM-3 Cells

| | | | | | | |
|---|---|---|---|---|---|---|
| 300 | nM | 1 | 2 | 3 | 1 | 2 |
| 50 | nM | 1 | 2 | 2 | 1 | 2 |
| 10 | nM | 1 | 1 | 2 | 1 | 2 |

PBMCs + JIM-3 Cells

| | | | | | | |
|---|---|---|---|---|---|---|
| 300 | nM | 1 | 1 | 3 | 1 | 2 |
| 50 | nM | 1 | 1 | 2 | 1 | 1 |
| 10 | nM | 1 | 1 | 2 | 1 | 1 |

PBMCs + JIM-3 Cells + NHSC

| | | | | | | |
|---|---|---|---|---|---|---|
| 300 | nM | 1 | 1 | 2 | 1 | 1 |
| 50 | nM | 1 | 1 | 1 | 1 | 1 |
| 10 | nM | 1 | 1 | 2 | 1 | 2 |

PBMCs + JIM-3 Cells + NHSC

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

PBMCs + JIM-3 Cells

| | | | | | | |
|---|---|---|---|---|---|---|
| 300 | nM | 1 | 1 | 2 | 1 | 1 |
| 50 | nM | 1 | 1 | 2 | 1 | 1 |
| 10 | nM | 1 | 1 | 1 | 1 | 1 |

PBMCs + JIM-3 Cells

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

PBMCs + JIM-3 Cells + NHSC

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

PBMCs + JIM-3 Cells + NHSC

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

CD, cluster of differentiation;
GPRC5D, G-protein-coupled receptor Family C Group 5 Member D;
hr, hour;
NHSC, normal human serum complement;
PBMC:T, peripheral blood mononuclear cell to target
Individual donor cytotoxicity and NK-cell activation (CD25⁺ and CD137⁺) fold change values observed for GC5B1552.AFU relative to vehicle control +/− 40% NHSC in PBMC coculture assays of GPRC5D⁺ JIM-3 cells after 24 and 48 hr (PBMC:T = 30:1, n = 5 healthy donors).

When evaluating individual PBMC donors cocultured with JIM-3 cells, up to 2-fold and up to 5-fold increases in cytotoxicity of JIM-3 cells were observed in the absence and presence of NHSC, respectively (see Table 18). Similarly, NK-cell activation among individual donors co-cultured with JIM-3 cells showed up to 3-fold and up to 2-fold increases in the absence and presence of NHSC, respectively (see Table 18).

### Cytokine and Chemokine Detection in PBMC Cocultures

IFN-γ, IL-1B, IL-2, IL-6, IL-10, TNF-α, MCP-1, MIP-1a, MIP-1B and IL-1RA levels were measured from supernatants collected from 5 healthy donor PBMCs co-cultured with JIM-3 cells and treated with GC5B1552.AFU in the presence or absence of 40% NHSC after 24 hours.

In PBMC coculture assays with JIM-3 cells, there were no significant (p<0.05) mean increases observed between GC5B1552.AFU and vehicle control in the absence of normal human serum complement after 24 hours. Mean and range values of GC5B1552.AFU and vehicle control are displayed in Table 19. With the addition of 40% NHSC, significant (p<0.05) mean differences between GC5B1552.AFU and vehicle control were observed in IL-10 levels only, which showed an inhibitory effect.

TABLE 19
Mean Secreted Cytokine and Chemokine Values Observed for GC5B1552.AFU
and Vehicle Control in PBMC Coculture Assays, in the Presence
or Absence of NHSC Using JIM-3 Target Cells.

| | GC5B1552.AFU | | | Vehicle Control |
|---|---|---|---|---|
| pg/mL | 300 nM | 50 nM | 10 nM | 0 nM |

PBMCs + JIM-3 Cells (no NHSC)

| | | | | | | | | |
|---|---|---|---|---|---|---|---|---|
| IFN-γ | 1049, | [3.02, 3646] | 767, | [2.73, 2042] | 736, | [1.71, 1635] | 67.3, | [1.65, 171] |
| IL-10 | 122, | [62.3, 171] | 111, | [61.3, 146] | 97.7, | [52.8, 143] | 171, | [75.1, 333] |
| IL-1RA | 1943, | [459, 5033] | 2061, | [488, 5109] | 2059, | [519, 5333] | 1728, | [473, 5009] |
| IL-1β | 2.47, | [0.2, 8.77] | 1.23, | [0.18, 3.46] | 1.96, | [0.19, 7.57] | 0.89, | [0.2, 2.11] |
| IL-2 | 7.30, | [0, 25.9] | 5.77, | [0.46, 17.1] | 4.76, | [0.33, 13.1] | 2.96, | [0.4, 6.43] |
| IL-6 | 32.5, | [1.93, 122] | 21.0, | [2.33, 69.4] | 26.6, | [3.42, 100] | 16.2, | [3.56, 47.0] |
| MCP-1 | 1750, | [1.24, 5888] | 1787, | [1.26, 5487] | 1475, | [1.22, 5127] | 701, | [1.20, 2066] |
| MIP-1α | 591, | [219, 1604] | 276, | [140, 360] | 253, | [114, 410] | 137, | [66.0, 213] |
| MIP-1β | 1124, | [21.5, 3933] | 625, | [22.4, 1947] | 417, | [25.7, 767] | 61.8, | [28.0, 125] |
| TNF-α | 39.1, | [1.00, 147] | 17.1, | [0.95, 48.1] | 9.37, | [1.17, 16.0] | 4.86, | [1.10, 7.31] |

PBMCs + JIM-3 Cells + NHSC

| | | | | | | | | |
|---|---|---|---|---|---|---|---|---|
| IFN-γ | 523, | [10.7, 1292] | 417, | [9.00, 1492] | 481, | [10.1, 1096] | 301, | [10.2, 689] |
| IL-10 | 278, | [213, 424] | 346, | [151, 737] | 363, | [260, 567] | 637, | [373, 950] |
| IL-1RA | 2133, | [182, 5221] | 1824, | [184, 5305] | 1925, | [176, 5425] | 1927, | [186, 5454] |
| IL-1β | 4.58, | [0.28, 18.6] | 2.28, | [0.25, 7.92] | 1.57, | [0.29, 3.69] | 1.53, | [0.25, 4.60] |
| IL-2 | 5.18, | [0.36, 15.9] | 6.00, | [0.067, 15.1] | 4.65, | [0, 13.4] | 3.87, | [0.046, 11.3] |
| IL-6 | 69.8, | [2.63, 307] | 32.6, | [2.47, 131] | 22.5, | [2.34, 74.4] | 18.4, | [3.25, 64.4] |
| MCP-1 | 3527, | [74.7, 5511] | 3129, | [68.8, 5401] | 3022, | [66.2, 5408] | 2819, | [62.5, 5335] |
| MIP-1α | 402, | [253, 755] | 431, | [178, 943] | 524, | [218, 1150] | 294, | [217, 453] |
| MIP-1β | 606, | [51.1, 1908] | 628, | [38.8, 2178] | 744, | [34.7, 2354] | 350, | [38.8, 1071] |
| TNF-α | 14.9, | [1.87, 53.0] | 14.7, | [1.42, 49.3] | 10.2, | [1.38, 26.7] | 6.96, | [1.73, 17.7] |

GPRC5D, G-protein-coupled receptor Family C Group 5 Member D; IFN, interferon; IL, interleukin; MCP, monocyte chemoattractant protein; MIP, macrophage inhibitory protein; NHSC, normal human serum complement; PBMC, peripheral blood mononuclear cell; PBMC:T, PBMC to target; RA, receptor antagonist; TNF, tumor necrosis factor.
Secreted cytokine and chemokine values (pg/mL) for GC5B1552.AFU and vehicle control in PBMC coculture assays, in the absence or presence of 40% NHSC using GPRC5D⁺ JIM-3 cells after 24 hours (PBMC:T ratio = 30:1, n = 5 healthy donors). Mean, [minimum, maximum] are shown.

When evaluating individual PBMC donors cocultured with JIM-3 cells, GC5B1552.AFU induced cytokine and chemokine levels >3-fold higher than vehicle control in IFN-γ (in 4 of 5 donors, 6-29-fold), IL-1B (in 2 of 5 donors, 3-4-fold), IL-2 (in 1 of 5 donors, 4-fold), IL-6 (in 1 of 5 donors, 3-fold), MCP-1 (in 3 of 5 donors, 3-4-fold), MIP-1a (in 3 of 5 donors, 3-24-fold), MIP-1B (in 4 of 5 donors, 4-31-fold) and TNF-α (in 3 of 5 donors, 3-20-fold) in the absence of 40% NHSC, and in IL-1B (in 1 of 5 donors, 4-fold), IL-2 (in 2 of 5 donors, 4-8-fold), IL-6 (in 1 of 5 donors, 5-fold), MIP-1a (in 1 of 5 donors, 3-fold), MIP-1B (in 2 of 5 donors, 3-fold), TNF-α (in 1 of 5 donors, 3-fold) in the presence of 40% NHSC (see Table 20).

TABLE 20
Individual Donor Cytokine and Chemokine Fold Change Values
Observed for GC5B1552.AFU Relative to Vehicle Control
in the Presence or Absence of Normal Human Serum Complement
in PBMC Coculture Assays of JIM-3 Target Cells.
PBMCs + JIM-3

| | Donor | Donor | Donor | Donor | Donor |
|---|---|---|---|---|---|
| GC5B1552.AFU | ID# | ID# | ID# | ID# | ID# |

IFN-γ

| | | | | | | |
|---|---|---|---|---|---|---|
| 300 | nM | 2 | 6 | 29 | 17 | 7 |
| 50 | nM | 2 | 14 | 16 | 17 | 7 |
| 10 | nM | 1 | 7 | 12 | 18 | 10 |

IL-10

| | | | | | | |
|---|---|---|---|---|---|---|
| 300 | nM | 1 | 1 | 1 | 1 | 1 |
| 50 | nM | 1 | 1 | 0 | 1 | 1 |
| 10 | nM | 1 | 1 | 0 | 1 | 1 |

IL-1RA

| | | | | | | |
|---|---|---|---|---|---|---|
| 300 | nM | 1 | 1 | 1 | 1 | 2 |
| 50 | nM | 1 | 2 | 1 | 2 | 1 |
| 10 | nM | 1 | 1 | 1 | 2 | 1 |

IL-1β

| | | | | | | |
|---|---|---|---|---|---|---|
| 300 | nM | 1 | 1 | 4 | 1 | 3 |
| 50 | nM | 1 | 1 | 2 | 1 | 1 |
| 10 | nM | 1 | 1 | 4 | 1 | 1 |

IL-2

| | | | | | | |
|---|---|---|---|---|---|---|
| 300 | nM | 0 | 1 | 4 | 2 | 1 |
| 50 | nM | 1 | 2 | 3 | 2 | 1 |
| 10 | nM | 1 | 1 | 2 | 2 | 2 |

IL-6

| | | | | | | |
|---|---|---|---|---|---|---|
| 300 | nM | 1 | 1 | 3 | 1 | 2 |
| 50 | nM | 1 | 1 | 1 | 2 | 1 |
| 10 | nM | 1 | 1 | 2 | 1 | 1 |

MCP-1

| | | | | | | |
|---|---|---|---|---|---|---|
| 300 | nM | 1 | 1 | 3 | 2 | 2 |
| 50 | nM | 1 | 3 | 3 | 4 | 1 |
| 10 | nM | 1 | 2 | 2 | 3 | 1 |

MIP-1α

| | | | | | | |
|---|---|---|---|---|---|---|
| 300 | nM | 1 | 3 | 24 | 2 | 4 |
| 50 | nM | 1 | 2 | 5 | 2 | 3 |
| 10 | nM | 1 | 2 | 3 | 2 | 3 |

MIP-1β

| | | | | | | |
|---|---|---|---|---|---|---|
| 300 | nM | 1 | 5 | 31 | 11 | 14 |
| 50 | nM | 1 | 4 | 16 | 11 | 8 |
| 10 | nM | 1 | 2 | 6 | 12 | 9 |

TNF-α

| | | | | | | |
|---|---|---|---|---|---|---|
| 300 | nM | 1 | 2 | 20 | 3 | 4 |
| 50 | nM | 1 | 2 | 7 | 2 | 3 |
| 10 | nM | 1 | 1 | 2 | 2 | 3 |

PBMCs + JIM-3 Cells + 40% NHSC

| | Donor | Donor | Donor | Donor | Donor |
|---|---|---|---|---|---|
| | ID# | ID# | ID# | ID# | ID# |
| GC5B1552.AFU | 60120 | 00220 | 01920 | 61920 | 92520 |

IFN-γ

| | | | | | | |
|---|---|---|---|---|---|---|
| 300 | nM | 1 | 1 | 2 | 1 | 2 |
| 50 | nM | 1 | 1 | 2 | 2 | 1 |
| 10 | nM | 1 | 1 | 2 | 2 | 2 |

IL-10

| | | | | | | |
|---|---|---|---|---|---|---|
| 300 | nM | 1 | 0 | 0 | 1 | 0 |
| 50 | nM | 1 | 0 | 0 | 1 | 0 |
| 10 | nM | 1 | 1 | 0 | 1 | 1 |

IL-1RA

| | | | | | | |
|---|---|---|---|---|---|---|
| 300 | nM | 1 | 1 | 1 | 1 | 2 |

IL-1RA

| | | | | | | |
|---|---|---|---|---|---|---|
| 50 | nM | 1 | 1 | 1 | 1 | 1 |
| 10 | nM | 1 | 1 | 1 | 1 | 1 |

IL-1β

| | | | | | | |
|---|---|---|---|---|---|---|
| 300 | nM | 1 | 1 | 4 | 1 | 2 |
| 50 | nM | 1 | 1 | 2 | 1 | 1 |
| 10 | nM | 1 | 2 | 1 | 1 | 1 |

IL-2

| | | | | | | |
|---|---|---|---|---|---|---|
| 300 | nM | 8 | 2 | 1 | 1 | 1 |
| 50 | nM | 1 | 1 | 1 | 1 | 4 |
| 10 | nM | 0 | 1 | 1 | 2 | 1 |

IL-6

| | | | | | | |
|---|---|---|---|---|---|---|
| 300 | nM | 1 | 2 | 5 | 1 | 2 |
| 50 | nM | 1 | 1 | 2 | 2 | 1 |
| 10 | nM | 1 | 2 | 1 | 1 | 1 |

MCP-1

| | | | | | | |
|---|---|---|---|---|---|---|
| 300 | nM | 1 | 1 | 1 | 1 | 2 |
| 50 | nM | 1 | 1 | 1 | 1 | 1 |
| 10 | nM | 1 | 1 | 1 | 1 | 1 |

MIP-1α

| | | | | | | |
|---|---|---|---|---|---|---|
| 300 | nM | 1 | 1 | 2 | 1 | 1 |
| 50 | nM | 1 | 1 | 2 | 1 | 1 |
| 10 | nM | 1 | 2 | 3 | 2 | 1 |

MIP-1β

| | | | | | | |
|---|---|---|---|---|---|---|
| 300 | nM | 1 | 1 | 2 | 2 | 2 |
| 50 | nM | 1 | 1 | 2 | 3 | 1 |
| 10 | nM | 1 | 3 | 2 | 2 | 2 |

TNF-α

| | | | | | | |
|---|---|---|---|---|---|---|
| 300 | nM | 1 | 1 | 3 | 1 | 1 |
| 50 | nM | 1 | 1 | 3 | 2 | 2 |
| 10 | nM | 1 | 2 | 2 | 2 | 1 |

GPRC5D, G-protein-coupled receptor Family C Group 5 Member D; IFN, interferon; IL, interleukin; MCP, monocyte chemoattractant protein; MIP, macrophage inhibitory protein; NHSC, normal human serum complement; PBMC, peripheral blood mononuclear cell; PBMC:T, PBMC to target; RA, receptor antagonist; TNF, tumor necrosis factor.
Individual donor cytokine and chemokine fold change values observed for GC5B1552.AFU relative to vehicle control in the absence or presence of 40% NHSC in PBMC coculture assays, using GPRC5D⁺ JIM-3 cells after 24 hours (PBMC:T ratio = 30:1, n = 5 healthy donors).

### Effect of GC5B1552.AFU in Whole-blood Coculture Assays Using JIM-3 Cells

### Cytotoxicity and NK-cell Activation in Whole-blood Cocultures

To evaluate the impact of GC5B1552.AFU in a physiologically relevant setting, in-vitro assays were conducted using GPRC5D⁺JIM-3 cells and healthy whole blood from 5 donors at a final NK: T ratio of 1:1 (Donor ID #s: 19, 196, 260 and 267) or 1.5:1 (Donor ID #: 110) after 24 and 48 hours.

At both time points, no significant (p<0.05) cytotoxicity of JIM-3 cells or NK-cell activation was observed with GC5B1552.AFU versus vehicle control. Mean and range values of GC5B1552.AFU and vehicle control are shown in Table 21. When evaluating individual whole blood donors cocultured with JIM-3 cells, a modest increase in percent cytotoxicity (up to 2-fold) as well as percent NK cell activation (up to 4-fold) was observed with GC5B1552.AFU when compared to vehicle control (Table 22).

TABLE 21
Percent Cytotoxicity and NK-cell Activation Values Observed for GC5B1552.AFU or
Vehicle Control in Whole-blood Cytotoxicity Assays Using JIM-3 Target Cells.

| Time | GC5B1552.AFU | | | Vehicle Control |
|---|---|---|---|---|
| (hrs) | 300 nM | 50 nM | 10 nM | 0 nM |

Cytotoxicity

| | | | | |
|---|---|---|---|---|
| 24 | 14.9, [10.6, 19.2] | 13.9, [8.19, 17.8] | 12.2, [8.98, 15.2] | 10.5, [6.95, 13.0] |
| 48 | 13.7, [9.90, 19.5] | 12.0, [7.25, 17.0] | 11.8, [7.48, 16.3] | 10.4, [6.71, 14.6] |

CD137⁺ NK-cell Activation

| | | | | |
|---|---|---|---|---|
| 24 | 8.08, [4.53, 15.0] | 9.60, [5.78, 17.6] | 11.5, [7.19, 21.4] | 6.12, [1.98, 16.8] |
| 48 | 4.35, [1.90, 6.23] | 5.55, [3.08, 7.97] | 6.24, [3.87, 9.39] | 4.25, [1.51, 7.73] |

CD25⁺ NK-cell Activation

| | | | | |
|---|---|---|---|---|
| 24 | 3.16, [1.51, 4.78] | 2.79, [1.71, 3.91] | 2.78, [2.09, 3.20] | 2.68, [1.54, 4.84] |
| 48 | 2.28, [0.76, 4.11] | 1.76, [0.86, 2.55] | 2.69, [0.89, 5.99] | 1.87, [0.65, 4.38] |

CD, cluster of differentiation; NK:T, NK cell to target; GPRC5D, G-protein-coupled receptor Family C Group 5 Member D; hrs, hours; NK, natural killer.
Percent cytotoxicity and NK-cell activation (expressed as CD25⁺ and CD137⁺ NK cells) values for GC5B1552.AFU or vehicle control in whole-blood coculture assays of GPRC5D⁺ JIM-3 cells after 24 hr and 48 hr, at a NK:T ratio of 1-1.5:1 (5 healthy donors). Mean, [minimum, maximum] are shown.

TABLE 22
Individual Donor Cytotoxicity and NK-cell Activation Fold Change
Values Observed for GC5B1552.AFU Relative to Vehicle Control
in Whole-Blood Cytotoxicity Assays Using JIM-3 Target Cells.

| | | | | | |
|---|---|---|---|---|---|
| | Donor | Donor | Donor | Donor | Donor |
| GC5B1552.AFU | ID# 19 | ID# 110 | ID# 196 | ID# 260 | ID# 267 |

Cytotoxicity
24 hr

| | | | | | | |
|---|---|---|---|---|---|---|
| 300 | nM | 2 | 1 | 1 | 1 | 1 |
| 50 | nM | 1 | 1 | 1 | 1 | 2 |
| 10 | nM | 2 | 1 | 1 | 1 | 1 |

Cytotoxicity
48 hrs

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

CD137⁺ NK-cell Activation
24 hrs

| | | | | | | |
|---|---|---|---|---|---|---|
| 300 | nM | 3 | 1 | 1 | 2 | 2 |
| 50 | nM | 3 | 1 | 1 | 3 | 3 |
| 10 | nM | 3 | 1 | 1 | 3 | 4 |

CD137⁺ NK-cell Activation
48 hrs

| | | | | | | |
|---|---|---|---|---|---|---|
| 300 | nM | 1 | 1 | 1 | 1 | 1 |
| 50 | nM | 2 | 1 | 1 | 2 | 2 |
| 10 | nM | 2 | 1 | 1 | 2 | 3 |

CD25⁺ NK-cell Activation
24 hrs

| | | | | | | |
|---|---|---|---|---|---|---|
| 300 | nM | 1 | 1 | 1 | 1 | 2 |
| 50 | nM | 1 | 1 | 1 | 1 | 2 |
| 10 | nM | 1 | 1 | 1 | 1 | 2 |

CD25⁺ NK-cell Activation
48 hrs

| | | | | | | |
|---|---|---|---|---|---|---|
| 300 | nM | 1 | 1 | 1 | 1 | 2 |
| 50 | nM | 1 | 1 | 1 | 1 | 1 |
| 10 | nM | 1 | 1 | 1 | 2 | 4 |

CD, cluster of differentiation; NK:T, NK cell to target; GPRC5D, G-protein-coupled receptor Family C Group 5 Member D; hrs, hours; NK, natural killer. Individual donor cytotoxicity and NK-cell activation (expressed as CD25⁺ and CD137⁺ NK cells) fold change values observed for GC5B1552.AFU relative to vehicle in whole-blood coculture assays of GPRC5D⁺ JIM-3 cells after 24 and 48 hrs, at a NK:T ratio of 1-1.5:1 (5 healthy donors).

### Cytokine Detection in Whole-blood Cocultures

Levels of IFN-γ, IL-1B, IL-2, IL-6, IL-10, TNF-α, MCP-1, MIP-1a, MIP-1B and IL-1RA in peripheral whole-blood coculture assay supernatants, using 5 healthy donors (Donor ID #s: 19, 110, 196, 260, 267) treated with GC5B1552.AFU were assessed with or without JIM-3 target cells after 24 hours.

In the absence of JIM-3 cells, there were no significant (p<0.05) mean increases in cytokine and chemokines levels observed in whole blood treated with GC5B1552.AFU when compared to vehicle control after 24 hours. Mean and range values of GC5B1552.AFU and vehicle control are displayed in Table 23. When evaluating individual whole blood donors, GC5B1552.AFU induced cytokine and chemokine levels ≥3-fold more than the vehicle control in in IL-1B (in 4 of 5 donors, 3-6-fold), IL-2 (in 2 of 5 donors, 3-4-fold), IL-6 (in 1 of 5 donors, 3-fold), MCP-1 (in 1 of 5 donors, 3-fold), MIP-1a (in 2 of 5 donors, 3-6-fold) and MIP-1ß (in 1 of 5 donors, 6-fold) (see Table 24).

In whole blood cocultured with JIM-3 cells, there were no significant (p<0.05) mean increases observed between GC5B1552.AFU and vehicle control after 24 hours. Mean and range values of GC5B1552.AFU and vehicle control are displayed in Table 23. When evaluating individual whole blood donors cocultured with JIM-3 cells, GC5B1552.AFU induced cytokine and chemokine levels >3-fold higher than the vehicle control in IFN-γ (in 4 of 5 donors, 3-13-fold), IL-1RA (in 2 of 5 donors, 3-4-fold), IL-1B (in 3 of 5 donors, 3-6-fold), IL-2 (in 3 of 5 donors, 3-4-fold), IL-6 (in 1 of 5 donors, 3-fold), MCP-1 (in 2 of 5 donors, 3-fold), MIP-1B (in 2 of 5 donors, 3-9-fold) and TNF-α (in 1 of 5 donors, 3-fold) after 24 hours (see Table 24).

TABLE 23
Mean Secreted Cytokine and Chemokine Values Observed for
GC5B1552.AFU and Vehicle Control in Whole-Blood Coculture
Assays, in the Presence or Absence of JIM-3 Target Cells.

| | GC5B1552.AFU | | | Vehicle Control |
|---|---|---|---|---|
| pg/mL | 300 nM | 50 nM | 10 nM | 0 nM |

Whole Blood Alone

| | | | | | | | | |
|---|---|---|---|---|---|---|---|---|
| IFN-γ | 41.9, | [9.41, 123] | 52.2, | [8.99, 166] | 58.6, | [9.39, 168] | 73.3, | [8.98, 186] |
| IL-10 | 1.34, | [0.52, 2.94] | 1.24, | [0.47, 2.30] | 1.13, | [0.54, 1.91] | 1.15, | [0.48, 1.95] |
| IL-1RA | 1104, | [364, 2270] | 1093, | [220, 2040] | 1048, | [268, 1600] | 1058, | [322, 1758] |
| IL-1β | 1.16, | [0.24, 2.20] | 1.72, | [0.25, 5.92] | 1.04, | [0.27, 2.05] | 0.84, | [0.29, 2.14] |
| IL-2 | 4.76, | [0.64, 12.0] | 2.52, | [0.56, 4.29] | 2.00, | [0.64, 5.60] | 2.50, | [0.89, 7.00] |
| IL-6 | 8.57, | [2.94, 20.3] | 9.63, | [2.03, 20.7] | 6.51, | [2.21, 13.5] | 6.07, | [2.78, 10.3] |
| MCP-1 | 1974, | [557, 5342] | 1772, | [253, 3458] | 1657, | [238, 3671] | 1411, | [339, 3459] |
| MIP-1α | 50.5, | [21.3, 128] | 50.1, | [15.4, 105] | 66.9, | [17.0, 205] | 36.0, | [19.6, 51.5] |
| MIP-1β | 385, | [61.2, 874] | 362, | [55.8, 986] | 356, | [61.4, 545] | 335, | [69.1, 755] |
| TNF-α | 5.99, | [2.03, 11.0] | 4.14, | [1.72, 7.51] | 3.81, | [1.32, 6.58] | 3.86, | [2.02, 6.45] |

Whole Blood + JIM-3 Cells

| | | | | | | | | |
|---|---|---|---|---|---|---|---|---|
| IFN-γ | 260, | [58.4, 401] | 359, | [80.4, 618] | 508, | [125, 919] | 119, | [29.7, 421] |
| IL-10 | 220, | [132, 354] | 229, | [146, 384] | 215, | [156, 338] | 241, | [158, 352] |
| IL-1RA | 2223, | [1309, 3827] | 2435, | [1130, 3943] | 2819, | [1658, 4527] | 1614, | [565, 2967] |
| IL-1β | 0.85, | [0.19, 1.61] | 0.66, | [0.23, 1.54] | 1.05, | [0.26, 2.81] | 0.56, | [0.15, 1.68] |
| IL-2 | 3.46, | [1.89, 7.73] | 3.39, | [2.43, 4.21] | 3.28, | [1.96, 5.03] | 2.88, | [1.03, 6.74] |
| IL-6 | 13.1, | [4.02, 36.7] | 12.8, | [4.00, 33.5] | 13.6, | [3.55, 32.7] | 17.2, | [2.89, 65.2] |
| MCP-1 | 4468, | [3683, 5105] | 4187, | [3083, 4988] | 4322, | [3703, 4756] | 2800, | [1365, 4460] |
| MIP-1α | 55.0, | [28.5, 111] | 62.0, | [32.6, 108] | 70.7, | [41.0, 98.4] | 50.6, | [25.8, 95.9] |
| MIP-1β | 415, | [49.1, 760] | 499, | [61.0, 997] | 618, | [61.3, 1318] | 248, | [42.3, 683] |
| TNF-α | 5.83, | [0.52, 17.0] | 6.46, | [1.35, 12.0] | 10.1, | [5.50, 14.3] | 7.82, | [2.66, 12.0] |

GPRC5D, G-protein-coupled receptor Family C Group 5 Member D; IFN, interferon; IL, interleukin; MCP, monocyte chemoattractant protein; MIP, macrophage inhibitory protein; NK, natural killer; NK:T, NK cell to target; RA, receptor antagonist; TNF, tumor necrosis factor.
Secreted cytokine and chemokine values (pg/mL) for GC5B1552.AFU and vehicle control from healthy donor peripheral whole blood coculture assays, in the presence or absence of GPRC5D⁺ JIM-3 cells after 24 hours (NK:T ratio = 1-1.5:1, n = 5 healthy donors). Mean, [minimum, maximum] are shown are shown.

TABLE 24
Individual Donor Cytokine and Chemokine Fold Change
Values Observed for GC5B1552.AFU Relative to Vehicle
Control in Whole-Blood Coculture Assays, in the
Presence or Absence of JIM-3 Target Cells.
Whole Blood Alone

| | Donor | Donor | Donor | Donor | Donor |
|---|---|---|---|---|---|
| GC5B1552.AFU | ID# 19 | ID# 110 | ID# 196 | ID# 260 | ID# 267 |

IFN-γ

| | | | | | | |
|---|---|---|---|---|---|---|
| 300 | nM | 0 | 1 | 1 | 1 | 1 |
| 50 | nM | 0 | 1 | 2 | 0 | 1 |
| 10 | nM | 1 | 1 | 1 | 1 | 1 |

IL-10

| | | | | | | |
|---|---|---|---|---|---|---|
| 300 | nM | 2 | 1 | 1 | 1 | 1 |
| 50 | nM | 1 | 2 | 1 | 1 | 1 |
| 10 | nM | 1 | 1 | 1 | 1 | 1 |

IL-1RA

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

IL-1β

| | | | | | | |
|---|---|---|---|---|---|---|
| 300 | nM | 3 | 1 | 1 | 2 | 3 |
| 50 | nM | 2 | 3 | 2 | 0 | 1 |
| 10 | nM | 2 | 1 | 6 | 1 | 1 |

IL-2

| | | | | | | |
|---|---|---|---|---|---|---|
| 300 | nM | 2 | 1 | 4 | 1 | 3 |
| 50 | nM | 1 | 1 | 2 | 0 | 3 |
| 10 | nM | 1 | 1 | 1 | 1 | 1 |

IL-6

| | | | | | | |
|---|---|---|---|---|---|---|
| 300 | nM | 2 | 1 | 1 | 1 | 2 |
| 50 | nM | 1 | 3 | 2 | 1 | 1 |
| 10 | nM | 1 | 1 | 2 | 1 | 0 |

MCP-1

| | | | | | | |
|---|---|---|---|---|---|---|
| 300 | nM | 2 | 1 | 1 | 1 | 2 |
| 50 | nM | 1 | 3 | 1 | 1 | 1 |
| 10 | nM | 1 | 1 | 2 | 2 | 1 |

MIP-1α

| | | | | | | |
|---|---|---|---|---|---|---|
| 300 | nM | 3 | 1 | 1 | 1 | 1 |
| 50 | nM | 1 | 2 | 1 | 1 | 1 |
| 10 | nM | 1 | 1 | 6 | 1 | 1 |

MIP-1β

| | | | | | | |
|---|---|---|---|---|---|---|
| 300 | nM | 2 | 1 | 1 | 1 | 1 |
| 50 | nM | 1 | 1 | 1 | 1 | 1 |
| 10 | nM | 1 | 1 | 6 | 1 | 1 |

TNF-α

| | | | | | | |
|---|---|---|---|---|---|---|
| 300 | nM | 2 | 2 | 2 | 1 | 1 |
| 50 | nM | 1 | 1 | 1 | 1 | 1 |
| 10 | nM | 1 | 1 | 2 | 1 | 1 |

Whole Blood + JIM-3 Cells

| | Donor | Donor | Donor | Donor | Donor |
|---|---|---|---|---|---|
| GC5B1552.AFU | ID# 19 | ID# 110 | ID# 196 | ID# 260 | ID# 267 |

IFN-γ

| | | | | | | |
|---|---|---|---|---|---|---|
| 300 | nM | 9 | 1 | 2 | 6 | 4 |
| 50 | nM | 7 | 1 | 3 | 9 | 6 |
| 10 | nM | 8 | 2 | 4 | 13 | 9 |

IL-10

| | | | | | | |
|---|---|---|---|---|---|---|
| 300 | nM | 1 | 1 | 1 | 1 | 1 |
| 50 | nM | 1 | 1 | 1 | 1 | 1 |
| 10 | nM | 1 | 1 | 1 | 1 | 1 |

IL-1RA

| | | | | | | |
|---|---|---|---|---|---|---|
| 300 | nM | 2 | 1 | 1 | 2 | 2 |
| 50 | nM | 1 | 1 | 1 | 3 | 2 |
| 10 | nM | 2 | 2 | 1 | 4 | 3 |

IL-1β

| | | | | | | |
|---|---|---|---|---|---|---|
| 300 | nM | 1 | 1 | 1 | 2 | 6 |
| 50 | nM | 1 | 2 | 3 | 1 | 1 |
| 10 | nM | 2 | 3 | 1 | 2 | 1 |

IL-2

| | | | | | | |
|---|---|---|---|---|---|---|
| 300 | nM | 2 | 2 | 0 | 2 | 2 |
| 50 | nM | 1 | 4 | 0 | 3 | 2 |
| 10 | nM | 1 | 3 | 0 | 2 | 4 |

IL-6

| | | | | | | |
|---|---|---|---|---|---|---|
| 300 | nM | 1 | 1 | 1 | 2 | 2 |
| 50 | nM | 1 | 1 | 1 | 2 | 2 |
| 10 | nM | 1 | 2 | 1 | 3 | 2 |

MCP-1

| | | | | | | |
|---|---|---|---|---|---|---|
| 300 | nM | 1 | 1 | 2 | 3 | 3 |
| 50 | nM | 1 | 1 | 2 | 3 | 2 |
| 10 | nM | 1 | 1 | 1 | 3 | 2 |

MIP-1α

| | | | | | | |
|---|---|---|---|---|---|---|
| 300 | nM | 1 | 1 | 1 | 1 | 2 |
| 50 | nM | 1 | 1 | 1 | 1 | 1 |
| 10 | nM | 1 | 2 | 1 | 2 | 2 |

MIP-1β

| | | | | | | |
|---|---|---|---|---|---|---|
| 300 | nM | 2 | 1 | 1 | 4 | 3 |
| 50 | nM | 2 | 1 | 1 | 8 | 3 |
| 10 | nM | 2 | 2 | 1 | 9 | 5 |

TNF-α

| | | | | | | |
|---|---|---|---|---|---|---|
| 300 | nM | 1 | 1 | 0 | 0 | 0 |
| 50 | nM | 1 | 1 | 0 | 1 | 1 |
| 10 | nM | 1 | 1 | 1 | 2 | 3 |

GPRC5D, G-protein-coupled receptor Family C Group 5 Member D; IFN, interferon; IL, interleukin; MCP, monocyte chemoattractant protein; MIP, macrophage inhibitory protein; NK, natural killer; NK:T, NK cell to target; RA, receptor antagonist; TNF, tumor necrosis factor.
Individual donor cytokine and chemokine fold change values observed for GC5B1552.AFU relative to vehicle control from healthy donor peripheral whole blood coculture assays, in the presence of absence of GPRC5D⁺ JIM-3 cells after 24 hours (NK:T ratio = 1-1.5:1, n = 5 healthy donors).

### CONCLUSIONS

In the PBMCs+JIM-3 co-culture assays, cytotoxicity of JIM-3 cells was observed, along with secreted cytokine and chemokines, which is consistent with the mechanisms of action of GC5B1552.AFU. A lower magnitude and incidence (among donors assessed) of cytokines and chemokines in PBMCs+JIM-3 (+40% NHSC) was observed. In the whole blood assay, a modest increase of some cytokines and chemokines was observed; the magnitude of the response was lower than that observed in the PBMC co-culture assay.

The following can be concluded from PBMC+JIM-3 coculture assays, using a GLP-grade batch of GC5B1552.AFU compared to vehicle control after 24 hours:

Significant cytotoxicity of JIM-3 cells was observed in the absence and presence of 40% NHSC; the presence of 40% NHSC resulted in more potent cytotoxicity than the absence of NHSC.

With some PBMC donors, an increase in IFN-γ, IL-1B, IL-2, IL-6, MCP-1, MIP-1a, MIP-1B, and TNF-α in the absence of NHSC, and in IL-1B, IL-2, IL-6, MIP-1a, MIP-1B, and TNF-α in the presence of NHSC was observed.

The following can be concluded from whole-blood coculture assays, using a GLP-grade batch of GC5B1552.AFU compared to vehicle control after 24 hours:

Modest cytotoxicity of JIM-3 cells and NK cell activation was observed in the 5 donors tested.

In the presence of JIM-3 cells, no significant increase in mean cytokine and chemokine levels was observed. With some donors, an increase in IFN-γ, IL-1RA, IL-1β, IL-2, IL-6, MCP-1, MIP-1β, and TNF-α was observed.

In the absence of JIM-3 cells, no significant increase in mean cytokine and chemokine levels was observed. With some donors, an increase in IL-1β, IL-2, IL-6, MCP-1, MIP-1a and MIP-1ß was observed.

### Example 12: In Vitro Pharmacologic Characterization of GC5β1552.AFU

### GPRC5D Expression in MM BMMC Plasma Cells and MM Cell Lines

The expression of GPRC5D was evaluated by flow cytometry in CD138⁺ (plasma cells) population of primary MM BMMCs (n=30) bone marrow mononuclear cells (BMMCs) (Table 25) and MM cell lines (Table 26). The JIM-3 and AMO-1 cell lines showed similar GPRC5D receptor density as MM patient samples.

TABLE 25
GPRC5D Percent Expression and Receptor
Density Values in CD138⁺ BMMC MM Samples

| Antigen | Percent CD138⁺ | Receptor density (ABC) |
|---|---|---|
| GPRC5D | 70.4 (±3.6) | 7,032 (±812) |

ABC, antibody binding capacity; BMMC, bone marrow mononuclear cells; CD, cluster of differentiation; GPRC5D, G-protein-coupled receptor Family C Group 5 Member D; MM, multiple myeloma; SEM, standard error of the mean.
Mean values ± SEM (n = 30) shown for GPRC5D in CD138⁺ (plasma cells) BMMC MM samples. Receptor density counts (in ABC) were quantified on live cells using Bangs Quantum ™ Simply Cellular ® quantitative cellular antigen expression kit.

TABLE 26
GPRC5D Percent Expression and Receptor
Density Values in MM Cell Lines

| Cell line | Receptor density (ABC) |
|---|---|
| OPM-2 | 148,938 |
| MM.1R | 168,781 |
| MM.1S | 65,913 |
| H929 | 47,351 |
| JIM-3 | 36,692 |
| AMO-1 | 29,017 |

ABC, antibody binding capacity; BMMC, bone marrow mononuclear cells; GPRC5D, G-protein-coupled receptor Family C Group 5 Member D; MM, multiple myeloma.
ABC values of GPRC5D are shown for 6 hematologic tumor cell lines. Receptor density counts (in ABC) were quantified on live cells using Bangs Quantum ™ Simply Cellular ® quantitative cellular antigen expression kit.

### GC5β1552.AFU-mediated Antibody-dependent Cellular Cytotoxicity (ADCC) of MM Cell Lines

An ADCC assay was used to evaluate the in vitro cytotoxicity of GC5β1552.AFU on MM.1R, H929, and AMO-1 MM cell lines using primary healthy donor human NK cells. An isotype control was used as a negative control. GC5β1552.AFU demonstrated concentration-dependent cytolytic activity of all the MM cells lines, with EC50 values of 0.005, 0.04, and 1.20 nM, for MM.1R, H929, and AMO-1 cells, respectively. GC5β1552.AFU showed no ADCC activity in a H929 GPRC5D KO cell line. NK cell activation was measured by CD137, which showed a similar pattern to MM cell cytotoxicity. Additionally, NK cells alone were treated with GC5β1552.AFU, isotype control, or anti-CD38 (positive control) evaluate NK cell fratricide. GC5β1552.AFU did not result in NK fratricide but the positive control anti-CD38 did, due to CD38 expression on NK cells.

### GC5β1552.AFU-mediated Complement-dependent Cytotoxicity (CDC) of MM Cell Lines

In vitro CDC activity of GC5β1552.AFU was evaluated in the MM.1R, JIM-3, and AMO-1 cell lines using a CellTiter-Glo® assay using 40% human serum. GC5β1552.AFU induced CDC on the cell lines tested, with EC₅₀ values of 8.77, 13.1, and 35.3 nM for MM.1R, JIM-3, and AMO-1, cells, respectively.

### GC5β1552.AFU-mediated Antibody-dependent Cellular Phagocytosis (ADCP)

The in vitro ADCP activity of GC5β1552.AFU in MM.1R and H929 cells was evaluated in an ADCP assay, using healthy donor isolated human monocytes induced into a M1-macrophage phenotype. GC5β1552.AFU induced ADCP against MM.1R and H929 with the same EC₅₀ value of 0.05 nM. The isotype control showed no ADCP activity in the cell lines tested. Additionally, GC5β1552.AFU did not induce any ADCP in a GPRC5D-H929 GPRC5D KO cell line.

### GC5β1552.AFU ADCC and CDC Effect Against MM Plasma Cells

Bone marrow mononuclear cells from patients with MM were used in ADCC and CDC assays to evaluate the cytotoxic activity of GC5β1552.AFU on the CD138⁺plasma cell population. All the samples expressed GPRC5D, which ranged in receptor density of 10,189 to 90,210 antibody binding capacity (ABC), with MM sample 6 having the highest GPRC5D expression (Table 27).

TABLE 27
Expression of GPRC5D CD138⁺ MM BMMCs
Used in ADCC and CDC Assays

| MM patient | GPRC5D ABC |
|---|---|
| Sample 1 | 63,592 |
| Sample 2 | 13,552 |
| Sample 6 | 90,210 |
| Sample 17 | 10,189 |

ABC, antibody binding capacity; ADCC, antibody-dependent cellular cytotoxicity; BMMC, bone marrow mononuclear cell; CD, cluster of differentiation; CDC, complement-dependent cytotoxicity; GPRC5D, G-protein-coupled receptor Family C Group 5 Member D; MM, multiple myeloma; NK, natural killer.
Receptor density counts (in ABC) were quantified on live cells using Bangs Quantum ™ Simply Cellular ® quantitative cellular antigen expression kit.

The cytotoxic activity of GC5β1552.AFU on MM BMMC CD138⁺plasma cells from 4 donors was evaluated using primary human NK cells derived from healthy donors in an ADCC assay and 40% human serum in a CDC assay. GC5β1552.AFU induced varying levels of ADCC with all 4 donors tested in a concentration-dependent manner and EC₅₀ values ranging between 0.007-0.64 nM and CDC activity in 2 of 4 MM samples tested, and EC₅₀ values of 35.5-40.5 nM.

The same MM donor samples were also evaluated in a CDC assay with GC5β1552.AFU along with 40% human serum. After 2 hours, cytotoxicity was evaluated like in the ADCC assay. Multiple myeloma patient sample 6 was the most sensitive and MM patient sample 17 showed minimal sensitivity to GC5β1552.AFU (Table 28). Samples 1 and 2 were not sensitive to CDC-mediated cytotoxicity by GC5β1552.AFU.

TABLE 28
EC₅₀ Values (nM) Observed for GC5B1552.AFU
From MM CD138⁺ cells in ADCC and CDC Assays

| | MM Sample# | | | |
|---|---|---|---|---|
| | Sample 1 | Sample 2 | Sample 6 | Sample 17 |
| ADCC EC₅₀ (nM) | 0.007 | 0.09 | 0.04 | 0.64 |
| CDC EC₅₀ (nM) | ND | ND | 40.5 | 35.5 |

ADCC, antibody-dependent cellular cytotoxicity; CD, cluster of differentiation; CDC, complement-dependent cytotoxicity; EC₅₀, 50% effective concentration; MM, multiple myeloma; ND, not determined.
GC5B1552.AFU viability EC₅₀ values from CD138⁺ MM patient samples (1, 2, 6, and 17) in ADCC and CDC assays are shown. EC₅₀ values were estimated using post-hoc estimation from the fitted model parameters.

1. An antibody or an antigen-binding fragment specifically binding to GPRC5D, comprising:
a. a heavy chain complementarity determining region 1 (CDR1), a heavy chain complementarity determining region 2 (CDR2), and a heavy chain complementarity determining region 3 (CDR3) comprising the amino acid sequences of SEQ ID NO: 6, 7, and 8, respectively; and
a light chain complementarity determining region 1 (CDR1), a light chain complementarity determining region 2 (CDR2), and a light chain complementarity determining region 3 (CDR3) comprising the amino acid sequences of SEQ ID NO: 9, 10, and 11, respectively;
b. a heavy chain CDR1, a heavy chain CDR2, and a heavy chain CDR3 comprising the amino acid sequences of SEQ ID NO: 12, 13, and 8, respectively; and
a light chain CDR1, a light chain CDR2, and a light chain CDR3 comprising the amino acid sequences of SEQ ID NO: 9, 10, and 11, respectively;
c. a heavy chain CDR1, a heavy chain CDR2, and a heavy chain CDR3 comprising the amino acid sequences of SEQ ID NO: 14, 15, and 8, respectively; and
a light chain CDR1, a light chain CDR2, and a light chain CDR3 comprising the amino acid sequences of SEQ ID NO: 9, 10, and 11, respectively;
d. a heavy chain CDR1, a heavy chain CDR2, and a heavy chain CDR3 comprising the amino acid sequences of SEQ ID NO: 16, 17, and 18, respectively; and
a light chain CDR1, a light chain CDR2, and a light chain CDR3 comprising the amino acid sequences of SEQ ID NO: 19, AAS, and 11, respectively; or
e. a heavy chain CDR1, a heavy chain CDR2, and a heavy chain CDR3 comprising the amino acid sequences of SEQ ID NO: 21, 22, and 23, respectively; and
a light chain CDR1, a light chain CDR2, and a light chain CDR3 comprising the amino acid sequences of SEQ ID NO: 24, 25, and 26, respectively.

2. The antibody or the antigen-binding fragment of claim 1, comprising:
a. a heavy chain variable region (VH) comprising an amino acid sequence that is at least 90%, 91%, 92%, 93%, 94%, 95%, 96%, 97%, 98%, 99%, or 100% identical to SEQ ID NO: 1; and
b. a light chain variable region (VL) comprising an amino acid sequence that is at least 90%, 91%, 92%, 93%, 94%, 95%, 96%, 97%, 98%, 99%, or 100% identical to SEQ ID NO: 2.

3.-6. (canceled)

7. The antibody or the antigen-binding fragment of claim 1, wherein the antibody or antigen-binding fragment is an IgG.

8. The antibody or the antigen-binding fragment of claim 1, wherein the antibody, or antigen-binding fragment comprises an IgG1 isotype Fc region.

9. The antibody or the antigen-binding fragment of claim 8, wherein the IgG1 isotype Fc region comprises one or more of the following:
a. K248E and T437R (RE) mutations as per the EU numbering system;
b. knob-into-hole (KiH) mutations; and
c. H435R and Y436F mutations per the EU numbering system.

10. The antibody or the antigen-binding fragment of claim 1, wherein the antibody or the antigen-binding fragment is afucosylated.

11. The antibody or the antigen-binding fragment of claim 10, wherein the antibody or the antigen-binding fragment has one or more of the following:
a. enhanced antibody-dependent cellular cytotoxicity (ADCC) activity;
b. enhanced complement-dependent cytotoxicity (CDC) activity;
c. antibody-dependent cellular phagocytosis (ADCP) activity.

12.-15. (canceled)

16. The antibody or the antigen-binding fragment thereof of claim 1, wherein the antibody or antigen-binding fragment thereof is monovalent.

17. The monovalent antibody or the antigen-binding fragment of claim 16, wherein the monovalent antibody or the antigen-binding fragment comprises an Fc domain and a Fab.

18. The monovalent antibody or the antigen-binding fragment of claim 16, wherein the monovalent antibody or the antigen-binding fragment comprises a first heavy chain (HCl), a second heavy chain (HC2), and a second light chain (LC2).

19. The monovalent antibody or the antigen-binding fragment of claim 18, comprising one or more of the following:
a. the HCl comprises an amino acid sequence that is at least 90%, 91%, 92%, 93%, 94%, 95%, 96%, 97%, 98%, 99%, or 100% identical to SEQ ID NO: 3;
b. the HC2 comprises an amino acid sequence that is at least 90%, 91%, 92%, 93%, 94%, 95%, 96%, 97%, 98%, 99%, or 100% identical to SEQ ID NO: 4; and
c. the LC2 comprises an amino acid sequence that is at least 90%, 91%, 92%, 93%, 94%, 95%, 96%, 97%, 98%, 99%, or 100% identical to SEQ ID NO: 5.

20.-25. (canceled)

26. The monovalent antibody or an antigen-binding fragment thereof of claim 19, wherein the monovalent antibody or the antigen-binding fragment thereof has enhanced antibody-dependent cellular cytotoxicity (ADCC) activity and enhanced complement-dependent cytotoxicity (CDC) as compared with a fucosylated divalent antibody or an antigen-binding fragment thereof without K248E and T437R (RE) mutations.

27. A synthetic polynucleotide encoding the antibody or the antigen-binding fragment of claim 1.

28. A vector comprising the synthetic polynucleotide of claim 27.

29. A host cell comprising the synthetic polynucleotide of claim 27, optionally wherein the host cell lacks fucosylation capability.

30. (canceled)

31. A pharmaceutical composition comprising the antibody; or the antigen-binding fragment of claim 1 and a pharmaceutically acceptable carrier.

32. A method for generating the antibody, the monovalent antibody, or the antigen-binding fragment specifically binding to GPRC5D, the method comprising growing the host cell of claim 29 under conditions to produce the antibody, the monovalent antibody, or the antigen-binding fragment.

33.-34. (canceled)

35. A method of inhibiting the growth or proliferation of a multiple myeloma, the method comprising administering to a subject the antibody, or the antigen-binding fragment of claim 1, optionally wherein the multiple myeloma is characterized by the presence of GPRC5D.

36. A method of treating a multiple myeloma, the method comprising administering to a subject in need thereof the antibody or the antigen-binding fragment of claim 1, to the subject for a time sufficient to treat the multiple myeloma, optionally wherein the multiple myeloma is characterized by the presence of GPRC5D.

37. (canceled)

38. A kit comprising one or more of the following:
a. the antibody or the antigen-binding fragment of claim 1;
b. the synthetic polynucleotide encoding the antibody or the antigen-binding fragment of claim 1; and
c. packaging for the same.

39.-40. (canceled)


SEQUENCE LISTING (29 sequences)

SEQ ID NO: 1 - protein, 120 aa, synthetic construct
QVQLVESGGGVVQPGRSLRLSCAASGFTFSNYGMHWVRQAPGKGLEWVAFIWYDGSNKYYADSVKGRFTISRDNSKNTLYLQMNSLRAEDTAVYYCAPITGYSTRWGDYWGQGTLVTVSS

SEQ ID NO: 2 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQRNTYPLTFGGGTKVEIK

SEQ ID NO: 3 - protein, 227 aa, synthetic construct
DKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYRQKSLSLSPGK

SEQ ID NO: 4 - protein, 450 aa, synthetic construct
QVQLVESGGGVVQPGRSLRLSCAASGFTFSNYGMHWVRQAPGKGLEWVAFIWYDGSNKYYADSVKGRFTISRDNSKNTLYLQMNSLRAEDTAVYYCAPITGYSTRWGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFRQKSLSLSPGK

SEQ ID NO: 5 - protein, 214 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQRNTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 10 aa, synthetic construct
GFTFSNYGMH

SEQ ID NO: 7 - protein, 10 aa, synthetic construct
FIWYDGSNKY

SEQ ID NO: 8 - protein, 11 aa, synthetic construct
ITGYSTRWGDY

SEQ ID NO: 9 - protein, 11 aa, synthetic construct
RASQGIRNDLG

SEQ ID NO: 10 - protein, 7 aa, synthetic construct
AASSLQS

SEQ ID NO: 11 - protein, 9 aa, synthetic construct
LQRNTYPLT

SEQ ID NO: 12 - protein, 5 aa, synthetic construct
NYGMH

SEQ ID NO: 13 - protein, 17 aa, synthetic construct
FIWYDGSNKYYADSVKG

SEQ ID NO: 14 - protein, 7 aa, synthetic construct
GFTFSNY

SEQ ID NO: 15 - protein, 6 aa, synthetic construct
WYDGSN

SEQ ID NO: 16 - protein, 8 aa, synthetic construct
GFTFSNYG

SEQ ID NO: 17 - protein, 8 aa, synthetic construct
IWYDGSNK

SEQ ID NO: 18 - protein, 13 aa, synthetic construct
APITGYSTRWGDY

SEQ ID NO: 19 - protein, 6 aa, synthetic construct
QGIRND

SEQ ID NO: 20 - skipped

SEQ ID NO: 21 - protein, 6 aa, synthetic construct
SNYGMH

SEQ ID NO: 22 - protein, 13 aa, synthetic construct
WVAFIWYDGSNKY

SEQ ID NO: 23 - protein, 12 aa, synthetic construct
APITGYSTRWGD

SEQ ID NO: 24 - protein, 7 aa, synthetic construct
RNDLGWY

SEQ ID NO: 25 - protein, 10 aa, synthetic construct
RLIYAASSLQ

SEQ ID NO: 26 - protein, 8 aa, synthetic construct
LQRNTYPL

SEQ ID NO: 27 - DNA, 750 bp, synthetic construct
  source 1..750: mol_type = other DNA
atggctagatccgcactgctcattctggctctgcttctgcttggactgttctctcctggagcatggggagacaaaactcacacttgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtccagatggcagcaggggaacgtcttctcatgctccgtgatgcatgaggctctgcacaaccactaccgccagaagtctctctccctgtctccgggaaaa

SEQ ID NO: 28 - DNA, 708 bp, synthetic construct
  source 1..708: mol_type = other DNA
atggccaggaagtccgctctgctcgctctggcacttctgcttctgggatttggacctgcttgggctgacatccagatgacccagtctccatcctctctgtccgccagcgtgggcgacagagtgaccatcacctgtagagcctcccagggcatcagaaacgacctcggctggtatcagcagaagcctggcaaggcccctaagcggctgatctacgctgcatcttctctgcagtctggcgtgccctccagattctccggatctggctctggcaccgagtttaccctgacaatctccagcctgcagcctgaggacttcgccacctactactgcctgcagcggaacacctatcctctgacctttggcggaggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 29 - DNA, 1416 bp, synthetic construct
  source 1..1416: mol_type = other DNA
atggccaggaagtccgctctgctcgctctggcacttctgcttctgggatttggacctgcttgggctcaggtgcagctggttgaatctggtggcggagtggtgcagcctggcagatctctgagactgtcttgtgccgcctccggcttcaccttctccaactacggaatgcactgggtccgacaggcccctggcaaaggattggagtgggtcgccttcatttggtacgacggctccaacaagtactacgccgactccgtgaagggcagattcaccatctctcgggacaactccaagaacaccctgtacctgcagatgaactccctgagagccgaggacaccgccgtgtactactgtgctcctatcaccggctactccaccagatggggcgattattggggccaaggcacactggtcacagtgtcctctgcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacttgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtccagatggcagcaggggaacgtcttctcatgctccgtgatgcatgaggctctgcacaacaggttccgccagaagtctctctccctgtctccgggaaaa